(19) United States
(12) Patent Application Publication
(10) Pub. No.: US 2002/0082225 A1
(43) Pub. Date: Jun. 27, 2002

(54) ENZYMATIC NUCLEIC ACID TREATMENT OF DISEASES OR CONDITIONS RELATED TO HEPATITIS C VIRUS INFECTION

(76) Inventors: LAWRENCE BLATT, BOULDER, CO (US); JAMES A. MCSWIGGEN, BOULDER, CO (US); BETH ROBERTS, FERDERAL HEIGHTS, CO (US); PAMELA A. PAVCO, LAFAYETTE, CO (US); DENNIS MACEJACK, ARVADA, CO (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 09/274,553

(22) Filed: Mar. 23, 1999

(51) Int. Cl. C12Q 1/68; C07H 21/04; A61K 31/70; A01N 43/04; C12N 15/74

(52) U.S. Cl. 514/44; 435/6; 435/475; 536/24.5

(57) ABSTRACT

Enzymatic nucleic acid molecules which modulate the expression and/or replication of hepatitis C.

This patent application claims priority to Blatt et al., U.S. Ser. No. (Not Yet Assigned), filed Feb. 24, 1999, Blatt et al., U.S. Ser. No. 60/100,842, filed Sep. 18, 1998, and McSwiggen et al., U.S. Ser. No. 60/083,217 filed Apr. 27, 1998, all of these earlier applications are entitled “ENZYMATIC NUCLEIC ACID TREATMENT OF DISEASES OR CONDITIONS RELATED TO HEPATITIS C VIRUS INFECTION”. Each of these applications are hereby incorporated by reference herein in their entirety including the drawings.

### BACKGROUND OF THE INVENTION

This invention relates to methods and reagents for the treatment of diseases or conditions relating to the hepatitic C virus infection.

The following is a discussion of relevant art, none of which is admitted to be prior art to the present invention.

In 1989, the HCV was determined to be an RNA virus and was identified as the causative agent of most non-A non-B viral Hepatitis (Choo et al., Science. 1989;

244:359-362). Unlike retroviruses such as HIV, HCV does not go though a DNA replication phase and no integrated forms of the viral genome into the host chromosome have been detected (Houghton et al., Hepatology 1991;14:381-388). Rather, replication of the coding (plus) strand is mediated by the production of a replicative (minus) strand leading to the generation of several copies of plus strand HCV RNA. The genome consists of a single, large, open-reading frame that is translated into a polyprotein (Kato et al., FEBS Letters. 1991; 280: 325-328). This polyprotein subsequently undergoes post-translational cleavage, producing several viral proteins (Leinbach et al., Virology. 1994: 204:163-169).

Examination of the 9.5-kilobase genome of HCV has demonstrated that the viral nucleic acid can mutate at a high rate (Smith et al., Mol. Evol. 1997 45:238-246). This rate of mutation has led to the evolution of several distinct genotypes of HCV that share approximately 70% sequence identity (Simmonds et al. J. Gen. Virol. 1994;75 :1053-061). It is important to note that these sequences are evolutionarily quite distant. For example, the genetic identity between humans and primates such as the chimpanzee is approximately 98%. In addition, it has been demonstrated that an HCV infection in an individual patient is composed of several distinct and evolving quasispecies that have 98% identity at the RNA level. Thus, the HCV genome is hypervariable and continuously changing. Although the HCV genome is hypervariable, there are 3 regions of the genome that are highly conserved. These conserved sequences occur in the 5′ and 3′ non-coding regions as well as the 5′-end of the core protein coding region and are thought to be vital for HCV RNA replication as well as translation of the HCV polyprotein. Thus, therapeutic agents that target these conserved HCV genomic regions may have a significant impact over a wide range of HCV genotypes. Moreover, it is unlikely that drug resistance will occur with ribozymes specific to conserved regions of the HCV genome. In contrast, therapeutic modalities that target inhibition of enzymes such as the viral proteases or helicase are likely to result in the selection for drug resistant strains since the RNA for these viral encoded enzymes is located in the hypervariable portion of the HCV genome.

After initial exposure to HCV, the patient will experience a transient rise in liver enzymes, which indicates that inflammatory processes are occurring (Alter et al, IN: Seeff L B, Lewis J H, eds. Current Perspectives in Hepatology. New York: Plenum Medical Book Co; 1989:83-89). This elevation in liver enzymes will occur at least 4 weeks after 25 the initial exposure and may last for up to two months (Farci et al., New England Journal of Medicine. 1991:325:98-104). Prior to the rise in liver enzymes, it is possible to detect HCV RNA in the patient's serum using RT-PCR analysis (Takahashi et al., American Journal of Gastroenterology. 1993:88:2:240-243). This stage of the disease is called the acute stage and usually goes undetected since 75% of patients with acute viral hepatitis from HCV infection are asymptomatic. The remaining 25% of these patients develop jaundice or other symptoms of hepatitis.

Acute HCV infection is a benign disease, however, and as many as 80% of acute HCV patients progress to chronic liver disease as evidenced by persistent elevation of serum alanine aminotransferase (ALT) levels and by continual presence of circulating HCV RNA (Sherlock, Lancet 1992; 339:802). The natural progression of chronic HCV infection over a 10 to 20 year period leads to cirrhosis in 20to50% of patients (Davis et al., Infectious Agents and Disease 1993;2:150:154) and progression of HCV infection to hepatocellular carcinoma has been well documented (Liang et al., Hepatology. 1993; 18:1326-1333; Tong et al., Western Journal of Medicine, 1994; Vol. 160, No. 2: 133-138). There have been no studies that have determined sub-populations that are most likely to progress to cirrhosis and/or hepatocellular carcinoma, thus all patients have equal risk of progression.

It is important to note that the survival for patients diagnosed with hepatocellular carcinoma is only 0.9 to 12.8 months from initial diagnosis (Takahashi et al., American Journal of Gastroenterology. 1993:88:2:240-243). Treatment of hepatocellular carcinoma with chemotherapeutic agents has not proven effective and only 10% of patients will benefit from surgery due to extensive tumor invasion of the liver (Trinchet et al., Presse Medicine. 1994:23:831-833). Given the aggressive nature of primary hepatocellular carcinoma, the only viable treatment alternative to surgery is liver transplantation (Pichlmayr et al., Hepatology. 1994:20:33S-40S).

Upon progression to cirrhosis, patients with chronic HCV infection present with clinical features, which are common to clinical cirrhosis regardless of the initial cause (D'Amico et al., Digestive Diseases and Sciences. 1986;31:5: 468-475). These clinical features may include: bleeding esophageal varices, ascites, jaundice, and encephalopathy (Zakim D, Boyer T D. Hepatology a textbook of liver disease. Second Edition Volume 1. 1990 W. B. Saunders Company. Philadelphia). In the early stages of cirrhosis, patients are classified as compensated meaning that although liver tissue damage has occurred, the patient's liver is still able to detoxify metabolites in the blood-stream. In addition, most patients with compensated liver disease are asymptomatic and the minority with symptoms report only minor symptoms such as dyspepsia and weakness. In the later stages of cirrhosis, patients are classified as decompensated meaning that their ability to detoxify metabolites in the bloodstream is diminished and it is at this stage that the clinical features described above will present.

In 1986, D'Amico et al. described the clinical manifestations and survival rates in 1155 patients with both alcoholic and viral associated cirrhosis (D'Amico supra). Of the 1155 patients, 435 (37%) had compensated disease although 70% were asymptomatic at the beginning of the study. The remaining 720 patients (63%) had decompensated liver disease with 78% presenting with a history of ascites, 31% with jaundice, 17% had bleeding and 16% had encephalopathy. Hepatocellular carcinoma was observed in six (0.5%) patients with compensated disease and in 30 (2.6%) patients with decompensated disease.

Over the course of six years, the patients with compensated cirrhosis developed clinical features of decompensated disease at a rate of 10% per year. In most cases, ascites was the first presentation of decompensation. In addition, hepatocellular carcinoma developed in 59 patients who initially presented with compensated disease by the end of the six-year study.

With respect to survival, the D'Amico study indicated that the five-year survival rate for all patients on the study was only 40%. The six-year survival rate for the patients who initially had compensated cirrhosis was 54% while the six-year survival rate for patients who initially presented with decompensated disease was only 21%. There were no significant differences in the survival rates between the patients who had alcoholic cirrhosis and the patients with viral related cirrhosis. The major causes of death for the patients in the D'Amico study were liver failure in 49%; hepatocellular carcinoma in 22%; and, bleeding in 13% (D'Amico supra).

Chronic Hepatitis C is a slowly progressing inflammatory disease of the liver, mediated by a virus (HCV) that can lead to cirrhosis, liver failure and/or hepatocellular carcinoma over a period of 10 to 20 years. In the US, it is estimated that infection with HCV accounts for 50,000 new cases of acute hepatitis in the United States each year (NIH Consensus Development Conference Statement on Management of Hepatitis C March 1997). The prevalence of HCV in the United States is estimated at 1.8% and the CDC places the number of chronically infected Americans at approximately 4.5 million people. The CDC also estimates that up to 10,000 deaths per year are caused by chronic HCV infection. The prevalence of HCV in the United States is estimated at 1.8% and the CDC places the number of chronically infected Americans at approximately 4.5 million people. The CDC also estimates that up to 10,000 deaths per year are caused by chronic HCV infection.

Numerous well controlled clinical trials using interferon (IFN-alpha) in the treatment of chronic HCV infection have demonstrated that treatment three times a week results in lowering of serum ALT values in approximately 50% (range 40% to 70%) of patients by the end of 6 months of therapy (Davis et al., New England Journal of Medicine 1989; 321:1501-1506; Marcellin et al., Hepatology. 1991; 13:393-397; Tong et al., Hepatology 1997:26:747-754; Tong et al., Hepatology 1997 26(6): 1640-1645). However, following cessation of interferon treatment, approximately 50% of the responding patients relapsed, resulting in a “durable” response rate as assessed by normalization of serum ALT concentrations of approximately 20 to 25%.

In recent years, direct measurement of the HCV RNA has become possible through use of either the branched-DNA or Reverse Transcriptase Polymerase Chain Reaction (RT-PCR) analysis. In general, the RT-PCR methodology is more sensitive and leads to more accurate assessment of the clinical course (Tong et al., supra). Studies that have examined six months of type 1 interferon therapy using changes in HCV RNA values as a clinical endpoint have demonstrated that up to 35% of patients will have a loss of HCV RNA by the end of therapy (Marcellin et al., supra). However, as with the ALT endpoint, about 50% of the patients relapse six months following cessation of therapy resulting in a durable virologic response of only 12% (Marcellin et al., supra). Studies that have examined 48 weeks of therapy have demonstrated that the sustained virological response is up to 25% (NIH consensus statement: 1997). Thus, standard of care for treatment of chronic HCV infection with type 1 interferon is now 48 weeks of therapy using changes in HCV RNA concentrations as the primary assessment of efficacy (Hoofnagle et al., New England Journal of Medicine 1997; 336(5) 347-356).

Side effects resulting from treatment with type 1 interferons can be divided into four general categories, which include 1. Influenza-like symptoms; 2. Neuropsychiatric; 3. Laboratory abnormalities; and, 4. Miscellaneous (Dusheiko et al., Journal of Viral Hepatitis. 1994:1:3-5). Examples of influenza-like symptoms include; fatigue, fever; myalgia; malaise; appetite loss; tachycardia; rigors; headache and arthralgias. The influenza-like symptoms are usually short-lived and tend to abate after the first four weeks of dosing (Dushieko et al., supra). Neuropsychiatric side effects include: irritability, apathy; mood changes; insomnia; cognitive changes and depression. The most important of these neuropsychiatric side effects is depression and patients who have a history of depression should not be given type 1 interferon. Laboratory abnormalities include; reduction in myeloid cells including granulocytes, platelets and to a lesser extent red blood cells. These changes in blood cell counts rarely lead to any significant clinical sequellae (Dushieko et al., supra). In addition, increases in triglyceride concentrations and elevations in serun alanine and aspartate aminotransferase concentration have been observed. Finally, thyroid abnormalities have been reported. These thyroid abnormalities are usually reversible after cessation of interferon therapy and can be controlled with appropriate medication while on therapy. Miscellaneous side effects include nausea; diarrhea; abdominal and back pain; pruritus; alopecia; and rhinorrhea. In general, most side effects will abate after 4 to 8 weeks of therapy (Dushieko et al., supra).

Welch et al., Gene Therapy 1996 3(11): 994-1001 describe in vitro an in vivo studies with two vector expressed hairpin ribozymes targeted against hepatitis C virus.

Sakamoto et al., J. Clinical Investigation 1996 98(12):2720-2728 describe intracellular cleavage of hepatitis C virus RNA and inhibition of viral protein translation by certain vector expressed hammerhead ribozymes.

Lieber et al., J. Virology 1996 70(12):8782-8791 describe elimination of hepatitis C virus RNA in infected human hepatocytes by adenovirus-mediated expression of certain hammerhead ribozymes.

Ohkawa et al., 1997, J Hepatology, 27; 78-84, describe in vitro cleavage of HCV RNA and inhibition of viral protein translation using certain in vitro transcribed hammerhead ribozymes.

Barber et al., International PCT Publication No. WO 97/32018, describe the use of an adenovirus vector to express certain anti-hepatitis C virus hairpin ribozymes.

Kay et al., International PCT Publication No. WO 96/18419, describe certain recombinant adenovirus vectors to express anti-HCV hammerhead ribozyme.

Yamada et al., Japanese Patent Application No. JP 07231 784 describe a specific poly-(L)-lysine conjugated hammerhead ribozyme targeted against HCV.

Draper, U.S. Pat. No. 5,610,054, descibes enzymatic nucleic acid molecule capable of inhibiting replication of HCV.

Alt et al., Hepatology 1995 22(3): 707-717, describe specific inhibition of hepatitis C viral gene expression by certain antisense phosphorothioate oligodeoxynucleotides.

### SUMMARY OF THE INVENTION

This invention relates to ribozymes, or enzymatic nucleic acid molecules, directed to cleave RNA species of hepatitis C virus (HCV) and/or encoded by the HCV. In particular, applicant describes the selection and function of ribozymes capable of specifically cleaving HCV RNA. Such ribozymes may be used to treat diseases associated with HCV infection.

Due to the high sequence variability of the HCV genome, selection of ribozymes for broad therapeutic applications would likely involve the conserved regions of the HCV genome. Specifically, the present invention describes hammerhead ribozymes that would cleave in the conserved regions of the HCV genome. A list of the thirty hammerhead ribozymes derived from the conserved regions (5′—Non Coding Region (NCR), 5′—end of core protein coding region, and 3′—NCR) of the HCV genome is shown in Table IV. In general, Applicant has found that enzymatic nucleic acid molecules that cleave sites located in the 5′ end of the HCV genome would block translation while ribozymes that cleave sites located in the 3′ end of the genome would block RNA replication. Approximately 50 HCV isolates have been identified and a sequence alignment of these isolates from genotypes 1a, 1b, 2a, 2b, 2c, 3a, 3b, 4a, 5a, and 6 was performed. These alignments were used by the Applicant to identify 30 hammerhead ribozymes sites within regions highly conserved between genotypes. Twenty three ribozyme sites were identified in regions of greatest homology within the conserved region. Therefore, one ribozyme can be designed to cleave all the different isolates of HCV. According to the Applicant, ribozymes designed against conserved regions of various HCV isolates will enable efficient inhibition of HCV replication in diverse patient populations and may ensure the effectiveness of the ribozymes against HCV quasispecies which evolve due to mutations in the non-conserved regions of the HCV genome.

By “inhibit” is meant that the activity of HCV or level of RNAs encoded by HCV genome is reduced below that observed in the absence of the nucleic acid, particularly, inhibition with ribozymes preferably is below that level observed in the presence of an inactive RNA molecule able to bind to the same site on the mRNA, but unable to cleave that RNA.

By “enzymatic nucleic acid” it is meant a nucleic acid molecule capable of catalyzing reactions including, but not limited to, site-specific cleavage and/or ligation of other nucleic acid molecules, cleavage of peptide and amide bonds, and trans-splicing. Such a molecule with endonuclease activity may have complementarity in a substrate binding region to a specified gene target, and also has an enzymatic activity that specifically cleaves RNA or DNA in that target. That is, the nucleic acid molecule with endonuclease activity is able to intramolecularly or intermolecularly cleave RNA or DNA and thereby inactivate a target RNA or DNA molecule. This complementarity functions to allow sufficient hybridization of the enzymatic RNA molecule to the target RNA or DNA to allow the cleavage to occur. 100% complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not limiting in the invention and those skilled in the art will recognize that all that is important in an enzymatic nucleic acid molecule of this invention is that it has a specific substrate binding site which is complementary to one or more of the target nucleic acid regions, and that it have nucleotide sequences within or surrounding that substrate binding site which impart a nucleic acid cleaving activity to the molecule.

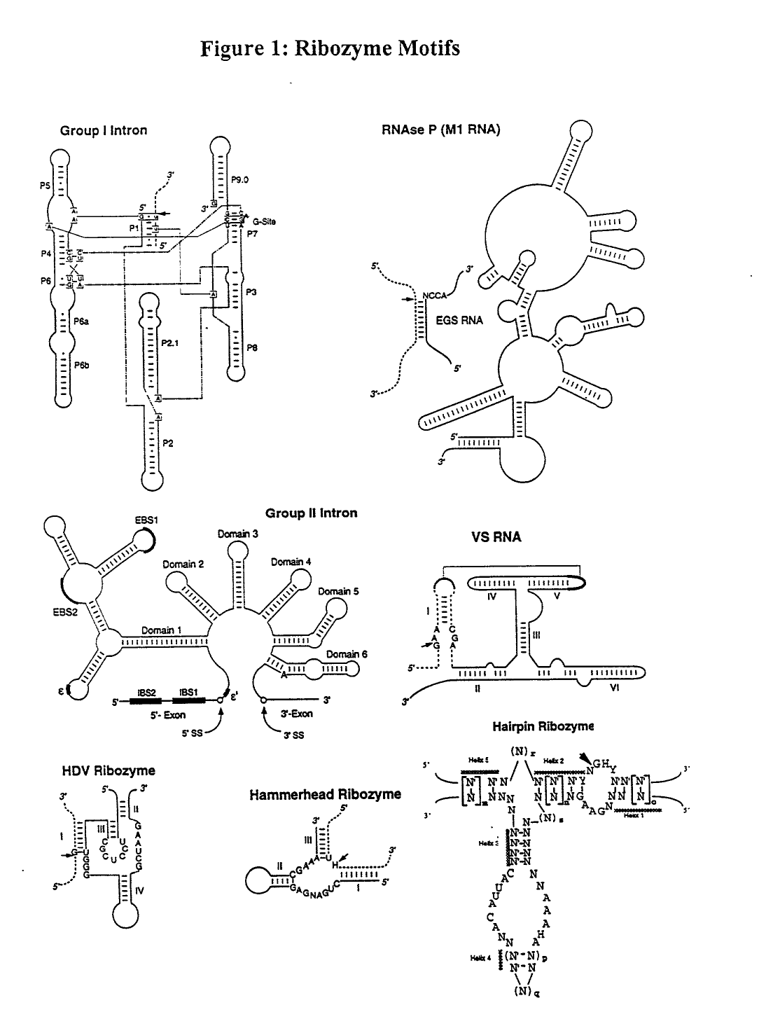

By “enzymatic portion” or “catalytic domain” is meant that portion/region of the ribozyme essential for cleavage of a nucleic acid substrate (for example see FIG. 1).

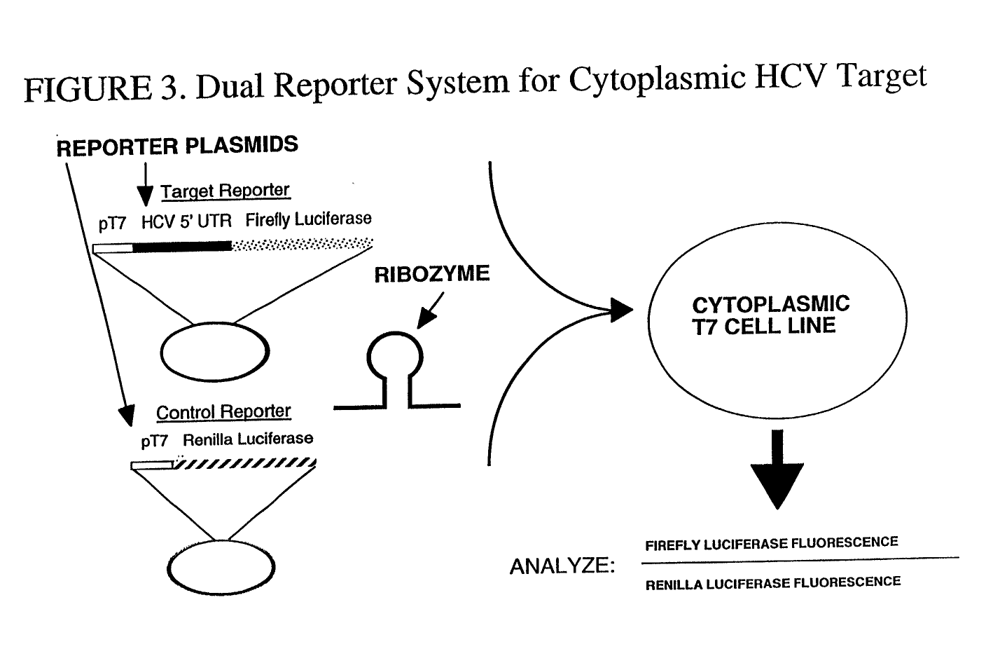

By “substrate binding arm” or “substrate binding domain” is meant that portion/region of a ribozyme which is complementary to (i.e., able to base-pair with) a portion of its substrate. Generally, such complementarity is 100%, but can be less if desired. For example, as few as 10 bases out of 14 may be base-paired. Such arms are shown generally in FIG. 1 and 3. That is, these arms contain sequences within a ribozyme which are intended to bring ribozyme and target RNA together through complementary base-pairing interactions. The ribozyme of the invention may have binding arms that are contiguous or non-contiguous and may be of varying lengths. The length of the binding arm(s) are preferably greater than or equal to four nucleotides; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (ie., the binding arms are of different length; e.g., six and three nucleotides; three and six nucleotides long; four and five nucleotides long; four and six nucleotides long; four and seven nucleotides long; and the like).

In one of the preferred embodiments of the inventions herein, the enzymatic 1 0 nucleic acid molecule is formed in a hammerhead or hairpin motif, but may also be formed in the motif of a hepatitis d virus, group I intron, group II intron or RNaseP RNA (in association with an RNA guide sequence) or Neurospora VS RNA. Examples of such hammerhead motifs are described by Dreyfus, supra, Rossi et al., 1992, AIDS Research and Human Retroviruses 8, 183; of hairpin motifs by Hampel et al., EP0360257, Hampel and Tritz, 1989 Biochemistry 28, 4929, Feldstein et al., 1989, Gene 82, 53, Haseloff and Gerlach, 1989, Gene, 82, 43, and Hampel et al., 1990 Nucleic Acids Res. 18, 299; of the hepatitis d virus motif is described by Perrotta and Been, 1992 Biochemistry 31, 16; of the RNaseP motif by Guerrier-Takada et al., 1983 Cell 35, 849; Forster and Altman, 1990, Science 249, 783; Li and Altman, 1996, Nucleic Acids Res. 24, 835; Neurospora VS RNA ribozyme motif is described by Collins (Saville and Collins, 1990 Cell 61, 685-696; Saville and Collins, 1991 Proc. Natl. Acad. Sci. USA 88, 8826-8830; Collins and Olive, 1993 Biochemistry 32, 2795-2799; Guo and Collins, 1995, EMBO. J 14, 363); Group II introns are described by Griffin et al., 1995, Chem. Biol. 2, 761; Michels and Pyle, 1995, Biochemistry 34, 2965; Pyle et al., International PCT Publication No. WO 96/22689; of the Group I intron by Cech et al., U.S. Pat. No. 4,987,071; and of DNAzyme motif by Chartrand et al., 1995, Nucleic Acids Research 23, 4092; Santoro et al., 1997, PNAS 94, 4262. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

By “equivalent” RNA to HCV is meant to include those naturally occurring RNA molecules associated with HCV infection in various animals, including human, rodent, primate, rabbit and pig. The equivalent RNA sequence also includes in addition to the coding region, regions such as 5′-untranslated region, 3′-untranslated region, introns, intron-exon junction and the like.

By “complementarity” is meant a nucleic acid that can form hydrogen bond(s) with another RNA sequence by either traditional Watson-Crick or other non-traditional types (for example, Hoogsteen type) of base-paired interactions.

In a preferred embodiment the invention provides a method for producing a class of enzymatic cleaving agents which exhibit a high degree of specificity for the RNA of a desired target. The enzymatic nucleic acid molecule is preferably targeted to a highly conserved sequence region of a target mRNAs encoding HCV proteins such that specific treatment of a disease or condition can be provided with either one or several enzymatic nucleic acids. Such enzymatic nucleic acid molecules can be delivered exogenously to specific cells as required. Alternatively, the ribozymes can be expressed from DNA/RNA vectors that are delivered to specific cells.

Such ribozymes are useful for the prevention of the diseases and conditions discussed above, and any other diseases or conditions that are related to the levels of HCV activity in a cell or tissue.

By “related” is meant that the inhibition of HCV RNAs and thus reduction in the level respective viral activity will relieve to some extent the symptoms of the disease or condition.

In preferred embodiments, the ribozymes have binding arms which are complementary to the target sequences in Tables IV-IX Examples of such ribozymes are also shown in Tables IV-IX. Examples of such ribozymes consist essentially of sequences defmed in these Tables. Other sequences may be present which do not interfere with such cleavage.

By “consists essentially of” is meant that the active ribozyme contains an enzymatic center or core equivalent to those in the examples, and binding arms able to bind MRNA such that cleavage at the target site occurs. Other sequences may be present which do not interfere with such cleavage.

Thus, in a first aspect, the invention features ribozymes that inhibit gene expression and/or viral replication. These chemically or enzymatically synthesized RNA molecules contain substrate binding domains that bind to accessible regions of their target mRNAs. The RNA molecules also contain domains that catalyze the cleavage of RNA. The RNA molecules are preferably ribozymes of the hammerhead or hairpin motif. Upon binding, the ribozymes cleave the target mRNAs, preventing translation and protein accumulation. In the absence of the expression of the target gene, HCV gene expression and/or replication is inhibited.

In a preferred embodiment, ribozymes are added directly, or can be complexed with cationic lipids, packaged within liposomes, or otherwise delivered to target cells. The nucleic acid or nucleic acid complexes can be locally administered to relevant tissues ex vivo, or in vivo through injection, infusion pump or stent, with or without their incorporation in biopolymers. In another preferred embodiment, the ribozyme is administered to the site of HCV activity (e.g., hepatocytes) in an appropriate liposomal vehicle.

In another aspect of the invention, ribozymes that cleave target molecules and inhibit HCV activity are expressed from transcription units inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target mRNA. Delivery of ribozyme expressing vectors could be systemic, such as by intravenous or intramuscular administration, by administration to target cells ex-planted from the patient followed by reintroduction into the patient, or by any other means that would allow for introduction into the desired target cell (for a review see Couture and Stinchcomb, 1996, TIG., 12, 510). In another aspect of the invention, ribozymes that cleave target molecules and inhibit viral replication are expressed from transcription units inserted into DNA, RNA, or viral vectors. Preferably, the recombinant vectors capable of expressing the ribozymes are locally delivered as described above, and transiently persist in smooth muscle cells. However, other mammalian cell vectors that direct the expression of RNA may be used for this purpose.

By “patient” is meant an organism which is a donor or recipient of explanted cells or the cells themselves. “Patient” also refers to an organism to which enzymatic nucleic acid molecules can be administered. Preferably, a patient is a mammal or mammalian cells. More preferably, a patient is a human or human cells.

By “vectors” is meant any nucleic acid- and/or viral-based technique used to deliver a desired nucleic acid.

These ribozymes, individually, or in combination or in conjunction with other drugs, can be used to treat diseases or conditions discussed above. For example, to treat a disease or condition associated with HCV levels, the patient may be treated, or other appropriate cells may be treated, as is evident to those skilled in the art.

Other features and advantages of the invention will be apparent from the following description of the preferred embodiments thereof, and from the claims.

### DESCRIPTION OF THE PREFERRED EMBODIMENTS

The drawings will first briefly be described.

Drawings:

FIG. 1 shows the secondary structure model for seven different classes of enzymatic nucleic acid molecules. Arrow indicates the site of cleavage. ------ indicate the target sequence. Lines interspersed with dots are meant to indicate tertiary interactions.—is meant to indicate base-paired interaction. Group I Intron: P1-P9.0 represent various stem-loop structures (Cech et al., 1994, Nature Struc. Bio., 1, 273). RNase P (MlRNA): EGS represents external guide sequence (Forster et al., 1990, Science, 249, 783; Pace et al., 1990, J Biol. Chem., 265, 3587). Group II Intron: 5′ SS means 5′ splice site; 3′ SS means 3′-splice site; IBS means intron binding site; EBS means exon binding site (Pyle et al., 1994, Biochemistry, 33, 2716). VS RNA: I-VI are meant to indicate six stem-loop structures; shaded regions are meant to indicate tertiary interaction (Collins, International PCT Publication No. WO 96/19577). HDV Ribozyme:: I-IV are meant to indicate four stem-loop structures (Been et al. U.S. Pat. No. 5,625,047). Hammerhead Ribozyme: 1-III are meant to indicate three stem-loop structures; stems I-III can be of any length and may be symmetrical or asymmetrical (Usman et al., 1996, Curr. Op. Struct. Bio., 1, 527). Hairpin Ribozyme: Helix 1, 4 and 5 can be of any length; Helix 2 is between 3 and 8 base-pairs long; Y is a pyrimidine; Helix 2 (H2) is provided with a least 4 base pairs (ie., n is 1, 2, 3 or 4) and helix 5 can be optionally provided of length 2 or more bases (preferably 3-20 bases, i.e., m is from 1-20 or more). Helix 2 and helix 5 may be covalently linked by one or more bases (i.e., r is≦1 base). Helix 1, 4 or 5 may also be extended by 2 or more base pairs (e.g., 4-20 base pairs) to stabilize the ribozyme structure, and preferably is a protein binding site. In each instance, each N and N′ independently is any normal or modified base and each dash represents a potential base-pairing interaction. These nucleotides may be modified at the sugar, base or phosphate. Complete base-pairing is not required in the helices, but is preferred. Helix 1 and 4 can be of any size (i.e., o and p is each independently from 0 to any number, e.g., 20) as long as some base-pairing is maintained. Essential bases are shown as specific bases in the structure, but those in the art will recognize that one or more may be modified chemically (abasic, base, sugar and/or phosphate modifications) or replaced with another base without significant effect. Helix 4 can be formed from two separate molecules, i.e., without a connecting loop. The connecting loop when present may be a ribonucleotide with or without modifications to its base, sugar or phosphate. “q” is≦2 bases. The connecting loop can also be replaced with a non-nucleotide linker molecule. H refers to bases A, U, or C. Y refers to pyrimidine bases. “______” refers to a covalent bond. (Burke et al., 1996, Nucleic Acids & Mol. Biol., 10, 129; Chowrira et al., U.S. Pat. No. 5,631,359).

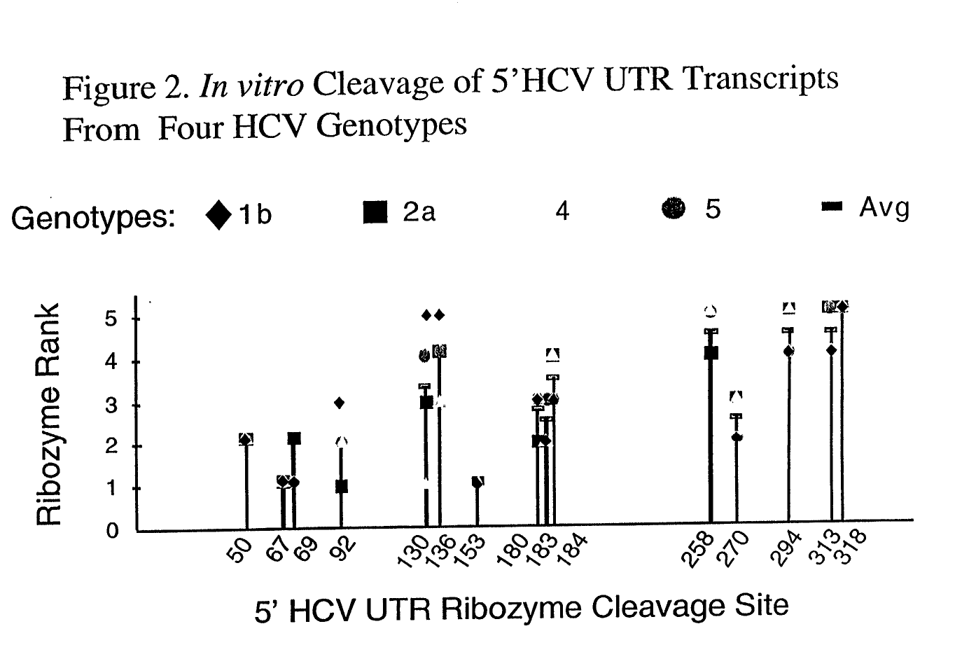

FIG. 2 is a graph displaying the ability of ribozymes targeting various sites within the conserved 5′ HCV UTR region to cleave the transcripts made from several genotypes.

FIG. 3 is a schematic representation of the Dual Reporter System utilized to demonstrate ribozyme mediated reduction of luciferase activity in cell culture.

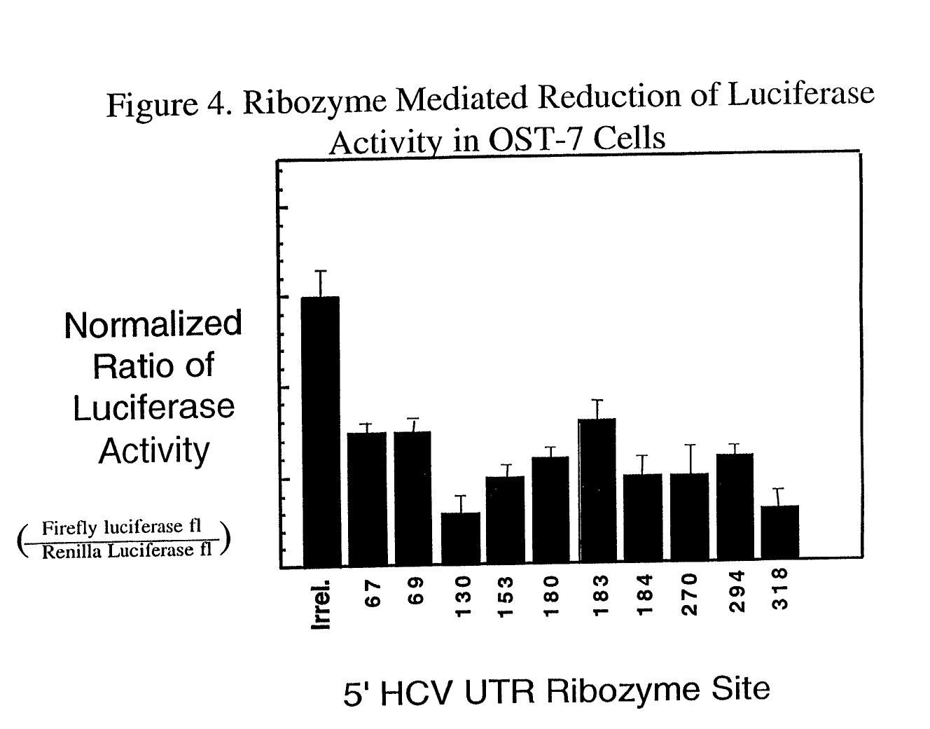

FIG. 4 is a graph demonstrating the ability of ribozymes to reduce luciferase activity in OST-7 cells.

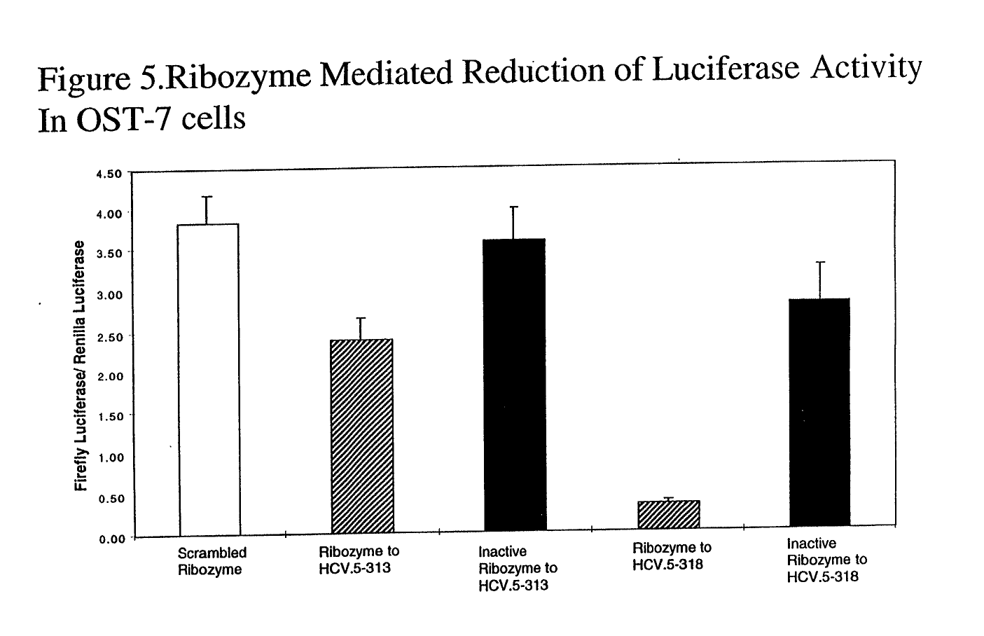

FIG. 5 is a graph demonstrating the ability of ribozymes targeting sites HCV.5-3 13 and HCV.5-318, to reduce luciferase activity in OST-7 cells compared to their inactive controls.

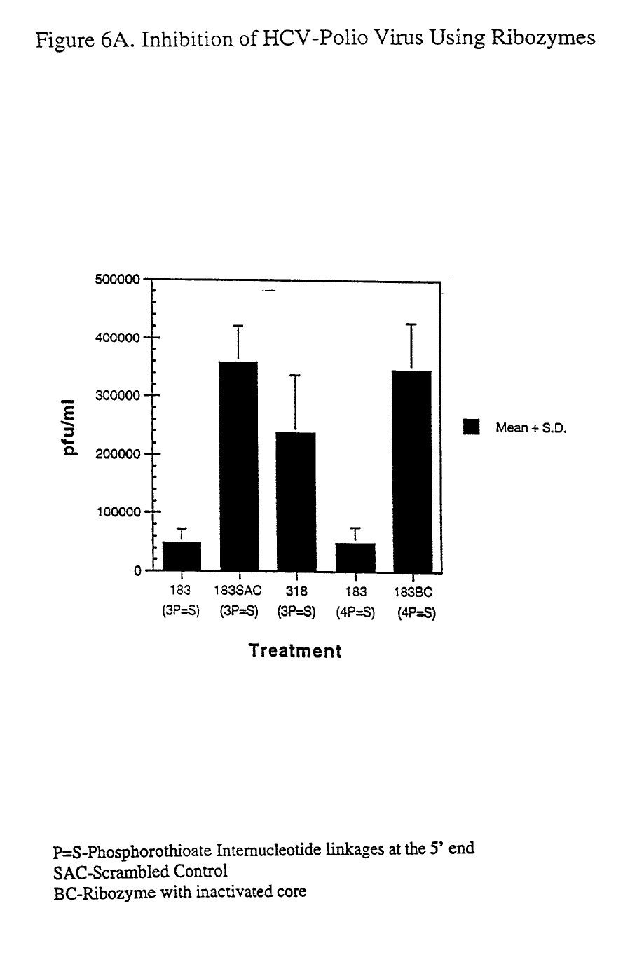

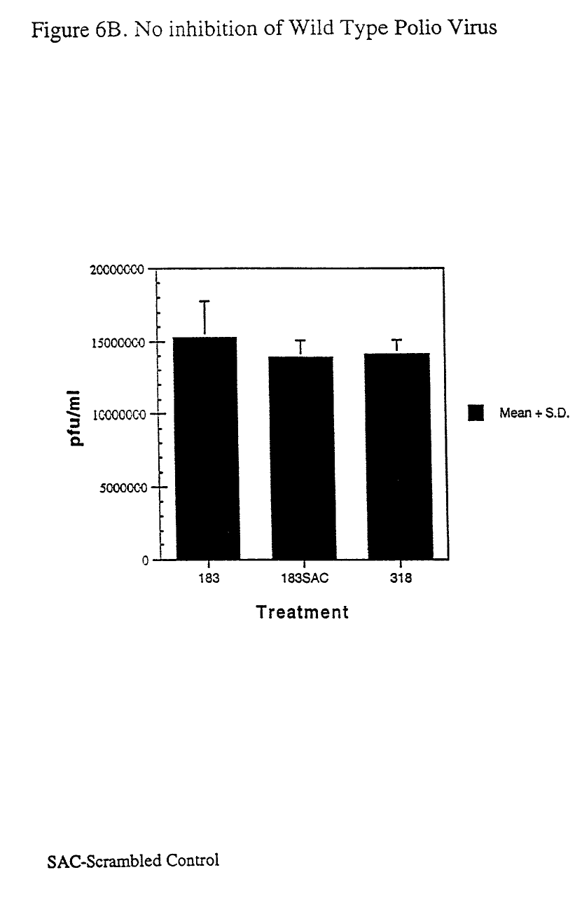

FIG. 6A is a bar graph demonstrating the effect of ribozyme treatment on HCV-Polio virus (PV) replication. HeLa cells in 96-well plates were infected with HCV-PV at a multiplicity of infection (MOI) of 0.1. Virus inoculum was then replaced with media containing 5% serum and ribozyme or control (200 nM), as indicated, complexed to a cationic lipid. After 24 hour cells were lysed 3 times by freeze/thaw and virus was quantified by plaque assay. Scrambled control (SAC), binding control (BAC), 3 P═S ribozymes, and 4 P═S ribozymes are indicated. Plaque forming units (pfu)/ml are shown as the mean of triplicate samples+standard deviation (S.D.).

FIG. 6B is a bar graph demonstrating the effect of ribozyme treatment on wild type PV replication. HeLa cells in 96-well plates were infected with wild type PV at an

MOI=0.05 for 30 minutes. All ribozymes contained 4P═S in (B). Plaque forming units (pfu)/ml are shown as the mean of triplicate samples+standard deviation (S.D.).

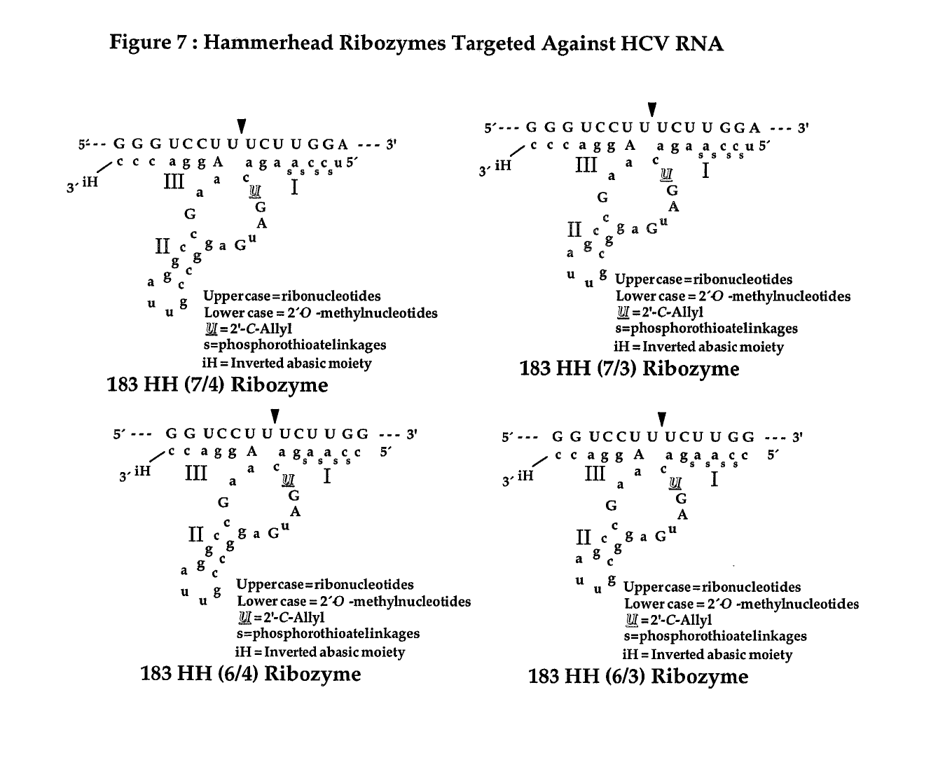

FIG. 7 is a schematic representation of various hammerhead ribozyme. constructs targeted against HCV RNA.

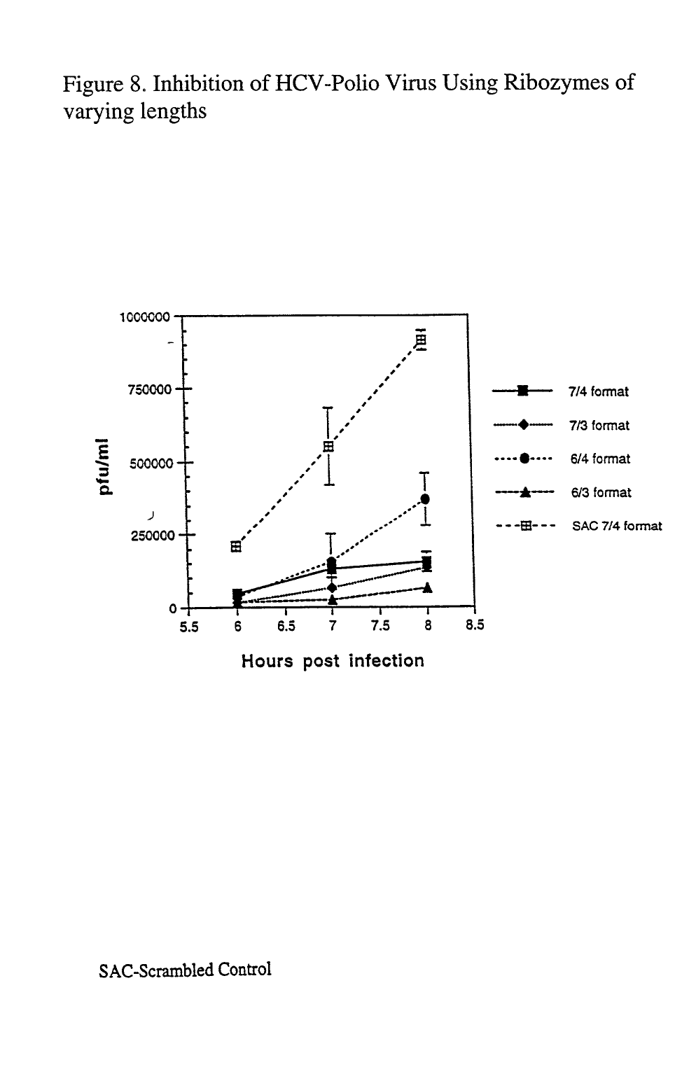

FIG. 8 is a graph demonstrating the effect of site 183 ribozyme treatment on a single round of HCV-PV infection. HeLa cells were infected with HCV-PV at an MOI=5 for 30 minutes prior to treatment with ribozymes or control. Cells were lysed after 6, 7, or 8 hours and virus was quantified by plaque assay. Ribozyme binding arm/stem II formats (7/4, 7/3, 6/4, 6/3) and scrambled control (SAC, 7/4 format) are indicated. All contained 4P═S stabilization. Results in pfu/ml are shown as the median of duplicate samples±range.

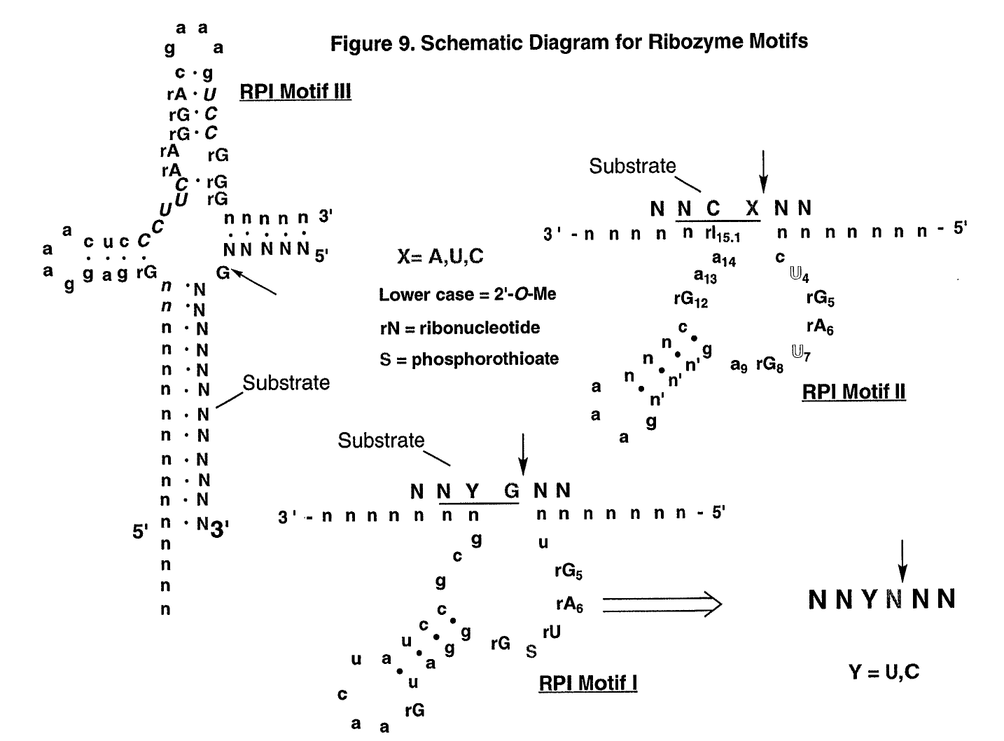

FIG. 9 shows the secondary structure models of three ribozyme motifs described in this application.

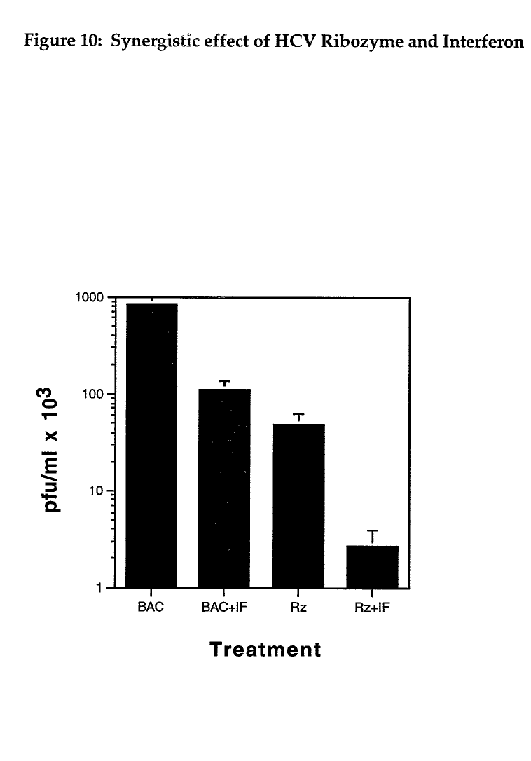

FIG. 10 shows the activity of anti-HCV ribozymes in combination with Interferon. Results in pfu/ml are shown as the median of duplicate samples±range. BAC, binding attenuated control molecule; IF, interferon; Rz, hammerhead ribozyme targeted to HCV site 183; pfu, plaque forming unit.

Ribozymes

Seven basic varieties of naturally-occurring enzymatic RNAs are known presently. In addition, several in vitro selection (evolution) strategies (Orgel, 1979, Proc. R. Soc. London, B 205, 435) have been used to evolve new nucleic acid catalysts capable of catalyzing cleavage and ligation of phosphodiester linkages (Joyce, 1989, Gene, 82, 83-87; Beaudry et al., 1992, Science 257, 635-641; Joyce, 1992, Scientific American 267, 90-97; Breaker et al., 1994, TIBTECH 12, 268; Bartel et al., 1993, Science 25 261:1411-1418; Szostak, 1993, TIBS 17, 89-93; Kumar et al., 1995, FASEB J, 9, 1183; Breaker, 1996, Curr. Op. Biotech., 7, 442; Santoro et al., 1997, Proc. Natl. Acad Sci., 94, 4262; Tang et al., 1997, RNA 3, 914; Nakamaye & Eckstein, 1994, supra; Long & Uhlenbeck, 1994, supra; Ishizaka et al., 1995, supra; Vaish et al., 1997, Biochemistry 36, 6495; all of these are incorporated by reference herein). Each can catalyze a series of reactions including the hydrolysis of phosphodiester bonds in trans (and thus can cleave other RNA molecules) under physiological conditions. Table I summarizes some of the characteristics of solne of these ribozymes. In general, enzymatic nucleic acids act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over other technologies, since the concentration of ribozyme necessary to affect a therapeutic treatment is lower. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base-pairing mechanism of binding to the target RNA, but also on the mechanism of target RNA cleavage. Single mismatches, or base-substitutions, near the site of cleavage can be chosen to completely eliminate catalytic activity of a ribozyme.

Nucleic acid molecules having an endonuclease enzymatic activity are able to repeatedly cleave other separate RNA molecules in a nucleotide base sequence-specific manner. Such enzymatic nucleic acid molecules can be targeted to virtually any RNA transcript, and efficient cleavage achieved in vitro (Zaug et al., 324, Nature 429 1986; Uhlenbeck, 1987 Nature 328, 596; Kim et al., 84 Proc. Natl. Acad. Sci. USA 8788, 1987; Dreyfus, 1988, Einstein Quart. J Bio. Med., 6, 92; Haseloff and Gerlach, 334 Nature 585, 1988; Cech, 260 JAMA 3030, 1988; and Jefferies et al., 17 Nucleic Acids Research 1371, 1989; Chartrand et al., 1995, Nucleic Acids Research 23, 4092; Santoro et al., 1997, PNAS 94,4262).

Because of their sequence-specificity, trans-cleaving ribozymes show promise as therapeutic agents for human disease (Usman & McSwiggen, 1995 Ann. Rep. Med. Chem. 30, 285-294; Christoffersen and Marr, 1995 J. Med. Chem. 38, 2023-2037). Ribozymes can be designed to cleave specific RNA targets within the background of cellular RNA. Such a cleavage event renders the RNA non-functional and abrogates protein expression from that RNA. In this manner, synthesis of a protein associated with a disease state can be selectively inhibited.

Ribozymes that cleave the specified sites in HCV RNAs represent a novel therapeutic approach to infection by the hepatitis C virus. Applicant indicates that ribozymes are able to inhibit the activity of HCV and that the catalytic activity of the ribozymes is required for their inhibitory effect. Those of ordinary skill in the art will find that it is clear from the examples described that other ribozymes that cleave HCV RNAs may be readily designed and are within the invention.

Target sites

Targets for useful ribozymes can be determined as disclosed in Draper et al., WO 93/23569; Sullivan et al., WO 93/23057; Thompson et al., WO 94/02595; Draper et al., WO 95/04818; McSwiggen et al., U.S. Pat. No. 5,525,468 and hereby incorporated by reference herein in totality. Rather than repeat the guidance provided in those documents here, below are provided specific examples of such methods, not limiting to those in the art. Ribozymes to such targets are designed as described in those applications and synthesized to be tested in vitro and in vivo, as also described. Such ribozymes can also be optimized and delivered as described therein.

The sequence of HCV RNAs were screened for optimal ribozyme target sites using a computer folding algorithm. Hammerhead or hairpin ribozyme cleavage sites were identified. These sites are shown in Tables IV-VIII (All sequences are 5′ to 3′ in the tables). The nucleotide base position is noted in the Tables as that site to be cleaved by the designated type of ribozyme. The nucleotide base position is noted in the Tables as that site to be cleaved by the designated type of ribozyme.

Because HCV RNAs are highly homologous in certain regions, some ribozyme target sites are also homologous (see Table IV and VIII). In this case, a single ribozyme will target different classes of HCV RNA. The advantage of one ribozyme that targets several classes of HCV RNA is clear, especially in cases where one or more of these RNAs may contribute to the disease state.

Hammerhead or hairpin ribozymes were designed that could bind and were individually analyzed by computer folding (Jaeger et al., 1989 Proc. Natl. Acad. Sci. USA, 86, 7706) to assess whether the ribozyme sequences fold into the appropriate secondary structure. Those ribozymes with unfavorable intramolecular interactions between the binding arms and the catalytic core are eliminated from consideration. Varying binding arm lengths can be chosen to optimize activity. Generally, at least 5 bases on each arm are able to bind to, or otherwise interact with, the target RNA. Ribozymes of the hammerhead or hairpin motif were designed to anneal to various sites in the mRNA message. The binding arms are complementary to the target site sequences described above.

Ribozyme Synthesis

Synthesis of nucleic acids greater than 100 nucleotides in length is difficult using automated methods, and the therapeutic cost of such molecules is prohibitive. In this invention, small nucleic acid motifs (e.g., hammerhead or the hairpin ribozymes) are used for exogenous delivery. The simple structure of these molecules increases the ability of the nucleic acid to invade targeted regions of the MRNA structure. However, these nucleic acid molecules can also be expressed within cells from eukaryotic promoters (e.g., Izant and Weintraub, 1985 Science 229, 345; McGarry and Lindquist, 1986 Proc. Natl. Acad. Sci. USA 83, 399; SullengerScanlon et al., 1991, Proc. Natl. Acad. Sci. USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad Sci. USA 89, 10802-6; Chen et al., 1992 Nucleic Acids Res., 20, 4581-9; Sarver et al., 1990 Science 247, 1222-1225; Thompson et al., 1995 Nucleic Acids Res. 23, 2259). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT W093/23569, and Sullivan et al., PCT W094/02595, both hereby incorporated in their totality by reference herein;

Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al., 1994 J. Biol. Chem. 269, 25856).

The ribozymes in the examples were chemically synthesized. The method of synthesis used follows the procedure for normal RNA synthesis as described in Usman et al., 1987 J. Am. Chem. Soc., 109, 7845; Scaringe et al., 1990 Nucleic Acids Res., 18, 5433; and Wincott et al., 1995 Nucleic Acids Res. 23, 2677-2684 and makes use of common nucleic acid protecting and coupling groups, such as dimethoxytrityl at the 5′-end, and phosphoramidites at the 3′-end. Small scale synthesis were conducted on a 394 Applied Biosystems, Inc. synthesizer using a modified 2.5 limol scale protocol with a 5 min coupling step for alkylsilyl protected nucleotides and 2.5 min coupling step for 2′-O-methylated nucleotides. Table II outlines the amounts, and the contact times, of the reagents used in the synthesis cycle. A 6.5-fold excess (163 μL of 0.1 M=16.3 μmol) of phosphoramidite and a 24-fold excess of S-ethyl tetrazole (238 AL of 0.25 M=59.5 μmol) relative to polymer-bound 5′-hydroxyl was used in each coupling cycle. Average coupling yields on the 394 Applied Biosystems, Inc. synthesizer, determined by colorimetric quantitation of the trityl fractions, were 97.5-99%. Other oligonucleotide synthesis reagents for the 394 Applied Biosystems, Inc. synthesizer : detritylation solution was 2% TCA in methylene chloride (ABI); capping was performed with 16% N-methyl imidazole in THF (ABI) and 10% acetic anhydride/10% 2,6-lutidine in THF (ABI); oxidation solution was 16.9 mM I2, 49 mM pyridine, 9% water in THF (Millipore). B & J Synthesis Grade acetonitrile was used directly from the reagent bottle. S-Ethyl tetrazole solution (0.25 M in acetonitrile) was made up from the solid obtained from American International Chemical, Inc.

Deprotection of the RNA was performed as follows. The polymer-bound oligoribonucleotide, trityl-off, was transferred from the synthesis column to a 4 mL glass screw top vial and suspended in a solution of methylamine (MA) at 65° C. for 10 min. After cooling to −20° C., the supernatant was removed from the polymer support. The support was washed three times with 1.0 InL of EtOH:MeCN:H₂O/3:1:1, vortexed and the supernatant was then added to the first supernatant. The combined supernatants, containing the oligoribonucleotide, were dried to a white powder.

The base-deprotected oligoribonucleotide was resuspended in anhydrous TEA.HF/NMP solution (250 gL of a solution of 1.5mL N-methylpyrrolidinone, 750 μL TEA and 1.0 mL TEA-3HF to provide a 1.4M HF concentration) and heated to 65° C. for 1.5 h. The resulting, fully deprotected, oligomer was quenched with 50 mM TEAB (9 mL) prior to anion exchange desalting.

For anion exchange desalting of the deprotected oligomer, the TEAB solution was loaded onto a Qiagen 500® anion exchange cartridge (Qiagen Inc.) that was prewashed with 50 mM TEAB (10 mL). After washing the loaded cartridge with 50 mM TEAB (10 mL), the RNA was eluted with 2 M TEAB (10 mL) and dried down to a white powder.

Inactive hammerhead ribozymes were synthesized by substituting switching the order of G₅A₆ and substituting a U for A₁₄(numbering from Hertel, K. J., et al, 1992, Nucleic Acids Res., 20, 3252). Inactive ribozymes were may also by synthesized by substituting a U for G5 and a U for A14. In some cases, the sequence of the substrate binding arms were randomized while the overall base composition was maintained.

The average stepwise coupling yields were>98% (Wincott et al., 1995 Nucleic Acids Res. 23, 2677-2684).

Hairpin ribozymes are synthesized in two parts and annealed to reconstruct the active ribozyme (Chowrira and Burke, 1992 Nucleic Acids Res., 20, 2835-2840). Ribozymes are also synthesized from DNA templates using bacteriophage T7 RNA polymerase (Milligan and Uhlenbeck, 1989, Methods Enzymol. 180, 51).

Ribozymes are modified to enhance stability and/or enhance catalytic activity by modification with nuclease resistant groups, for example, 2′-amino, 2′-C-allyl, 2′-flouro, 2′-O-methyl, 2′-H, nucleotide base modifications (for a review see Usman and Cedergren, 1992 TIBS 17, 34; Usman et al., 1994 Nucleic Acids Symp. Ser. 31, 163; Burgin et al. 1996 Biochemistry 6, 14090).

Ribozymes were purified by gel electrophoresis using general methods or are purified by high pressure liquid chromatography (HPLC; See Stinchcomb et al., International PCT Publication No. WO 95/23225, the totality of which is hereby incorporated herein by reference) and are resuspended in water.

The sequences of the ribozymes that are chemically synthesized, useful in this study, are shown in Tables IV-IX. Those in the art will recognize that these sequences are representative only of many more such sequences where the enzymatic portion of the ribozyme (all but the binding arms) is altered to affect activity. For example, stem-loop II sequence of hammerhead ribozymes can be altered (substitution, deletion, and/or insertion) to contain any sequences provided a minimum of two base-paired stem structure can form. Similarly, stem-loop IV sequence of hairpin ribozymes, can be altered (substitution, deletion, and/or insertion) to contain any sequence, provided a minimum of two base-paired stem structure can form. Preferably, no more than 200 bases are inserted at these locations. The sequences listed in Tables IV-IX may be formed of ribonucleotides or other nucleotides or non-nucleotides. Such ribozymes (which have enzymatic activity) are equivalent to the ribozymes described specifically in the Tables.

Optimizing Ribozyme Activity

Catalytic activity of the ribozymes described in the instant invention can be optimized as described by Draper et al., supra. The details will not be repeated here, but include altering the length of the ribozyme binding arms, or chemically synthesizing ribozymes with modifications (base, sugar and/or phosphate) that prevent their degradation by serum ribonucleases and/or enhance their enzymatic activity (see e.g., Eckstein et al., International Publication No. WO 92/07065; Perrault et al., 1990 Nature 344, 565; Pieken et al., 1991 Science 253, 314; Usman and Cedergren, 1992 Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of enzymatic RNA molecules). Modifications which enhance their efficacy in cells, and removal of bases from stem loop structures to shorten RNA synthesis times and reduce chemical requirements are desired. (All these publications are hereby incorporated by reference herein).

There are several examples in the art describing sugar and phosphate modifications that can be introduced into enzymatic nucleic acid molecules without significantly effecting catalysis and with significant enhancement in their nuclease stability and efficacy. Ribozymes are modified to enhance stability and/or enhance catalytic activity by modification with nuclease resistant groups, for example, 2′-amino, 2′-C-allyl, 2′-flouro, 2′-O-methyl, 2′-H, nucleotide base modifications (for a review see Usman and Cedergren, 1992 TIBS 17, 34; Usman et al. 1994 Nucleic Acids Symp. Ser. 31, 163; Burgin et al., 1996 Biochemistry 35, 14090). Sugar modification of enzymatic nucleic acid molecules have been extensively described in the art (see Eckstein et al., International Publication PCT No. WO 92/07065; Perrault et al. Nature 1990, 344, 565-568; Pieken et al. Science 1991, 253, 314-317; Usman and Cedergren, Trends in Biochem. Sci 1992, 17, 334-339; Usman et al. International Publication PCT No. WO 93/15187; Sproat, U.S. Pat. No. 5,334,711 and Beigelman et al., 1995 J. Biol. Chem. 270, 25702; all of the references are hereby incorporated in their totality by reference herein). Such publications describe general methods and strategies to determine the location of incorporation of sugar, base and/or phosphate modifications and the like into ribozymes without inhibiting catalysis, and are incorporated by reference herein. In view of such teachings, similar modifications can be used as described herein to modify the nucleic acid catalysts of the instant invention.

Nucleic acid catalysts having chemical modifications which maintain or enhance enzymatic activity are provided. Such nucleic acid is also generally more resistant to nucleases than unmodified nucleic acid. Thus, in a cell and/or in vivo the activity may not be significantly lowered. As exemplified herein such ribozymes are useful in a cell and/or in vivo even if activity over all is reduced 10 fold (Burgin et al., 1996, Biochemistry, 35, 14090). Such ribozymes herein are said to “maintain” the enzymatic activity on all RNA ribozyme.

Therapeutic ribozymes delivered exogenously must optimally be stable within cells until translation of the target RNA has been inhibited long enough to reduce the levels of the undesirable protein. This period of time varies between hours to days depending upon the disease state. Clearly, ribozymes must be resistant to nucleases in order to function as effective intracellular therapeutic agents. Improvements in the chemical synthesis of RNA (Wincott et al., 1995 Nucleic Acids Res. 23, 2677; incorporated by reference herein) have expanded the ability to modify ribozymes by introducing nucleotide modifications to enhance their nuclease stability as described above.

By “nucleotide” as used herein is as recognized in the art to include natural bases (standard), and modified bases well known in the art. Such bases are generally located at the 1′ position of a sugar moiety. Nucleotide generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other ; see for example, Usman and McSwiggen, supra; Eckstein et al., International PCT Publication No. WO 92/07065; Usman et al., International PCT Publication No. WO 93/15187; all hereby incorporated by reference herein). There are several examples of modified nucleic acid bases known in the art and has recently been summarized by Limbach et al., 1994, Nucleic Acids Res. 22, 2183. Some of the non-limiting examples of base modifications that can be introduced into enzymatic nucleic acids without significantly effecting their catalytic activity include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine) and others (Burgin et al., 1996, Biochemistry, 35, 14090). By “modified bases” in this aspect is meant nucleotide bases other than adenine, guanine, cytosine and uracil at 1′ position or their equivalents; such bases may be used within the catalytic core of the enzyme and/or in the substrate-binding regions.

By “abasic” is meant sugar moieties lacking a base or having other chemical groups in place of base at the 1′ position.

By “unmodified nucleoside” is meant one of the bases adenine, cytosine, guanine, uracil joined to the 1′ carbon of beta-D-ribo-furanose.

By “modified nucleoside” is meant any nucleotide base which contains a modification in the chemical structure of an unmodified nucleotide base, sugar and/or phosphate.

Various modifications to ribozyme structure can be made to enhance the utility of ribozymes. Such modifications will enhance shelf-life, half-life in vitro, stability, and ease of introduction of such ribozymes to the target site, e.g., to enhance penetration of cellular membranes, and confer the ability to recognize and bind to targeted cells.

Administration of Ribozymes

Sullivan et al., PCT WO 94/02595, describes the general methods for delivery of enzymatic RNA molecules . Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination is locally delivered by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Sullivan et al., supra and Draper et al, PCT W093/23569 which have been incorporated by reference herein.

The molecules of the instant invention can be used as pharmaceutical agents. Pharmaceutical agents prevent, inhibit the occurrence, or treat (alleviate a symptom to some extent, preferably all of the symptoms) of a disease state in a patient.

The negatively charged polynucleotides of the invention can be administered (e.g., RNA, DNA or protein) and introduced into a patient by any standard means, with or without stabilizers, buffers, and the like, to form a pharmaceutical composition. When it is desired to use a lipid or liposome delivery mechanism, standard protocols for formulation can be followed. The compositions of the present invention may also be formulated and used as tablets, capsules or elixirs for oral administration; suppositories for rectal administration; sterile solutions; suspensions for injectable administration; and the like.

The present invention also includes pharmaceutically acceptable formulations of the compounds described. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, preferably a human. Suitable forms, in part, depend upon the use or the route of entry, for example oral, transdermal, or by injection. Such forms should not prevent the composition or formulation to reach a target cell (i.e., a cell to which the negatively charged polymer is desired to be delivered to). For example, pharmacological compositions injected into the blood stream should be soluble. Other factors are known in the art, and include considerations such as toxicity and forms which prevent the composition or formulation from exerting its effect.

By “systemic administration” is meant in vivo systemic absorption or accumulation of drugs in the blood stream followed by distribution throughout the entire body. Administration routes which lead to systemic absorption include, without limitations: intravenous, subcutaneous, intraperitoneal, inhalation, oral, intrapulmonary and intramuscular. Each of these administration routes expose the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant invention can potentially localize the drug, for example, in certain tissue types, such as the tissues of the reticular endothelial system (RES). A liposome formulation which can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as the HCV infected liver cells.

The invention also features the use of a composition comprising surface-modified liposomes containing poly (ethylene glycol) lipids (PEG-modified, or long-circulating liposomes or stealth liposomes). These formulations offer an method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al. Chem. Rev. 1995, 95, 2601-2627; Ishiwataet al., Chem. Pharm. Bull. 1995, 43, 1005-1011 ). Such liposomes have been shown to accumulate selectively in tumors, presumably by extravasation and capture in the neovascularized target tissues (Lasic et al., Science 1995, 267, 1275-1276; Oku et al., 1995, Biochim. Biophys. Acta, 1238, 86-90). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 1995, 42, 24864-24870; Choi et al., International PCT Publication No. WO 96/10391; Ansell et al., International PCT Publication No. WO 96/10390; Holland et al., International PCT Publication No. WO 96/10392; all of these are incorporated by reference herein). All of these references are incorporated by reference herein.

In addition other cationic molecules may also be utilized to deliver the molecules of the present invention. For example, ribozymes may be conjugated to glycosylated poly(L-lysine) which has been shown to enhance localization of antisense oligonucleotides into the liver (Nakazono et al., 1996, Hepatology 23, 1297-1303; Nahato et al., 1997, Biochem Pharm. 53, 887-895). Glycosylated poly(L-lysine) may be covently attached to the enzymatic nucleic acid or be bound to enzymatic nucleic acid through electrostatic interaction.

The present invention also includes compositions prepared for storage or administration which include a pharmaceutically effective amount of the desired compounds in a pharmaceutically acceptable carrier or diluent. Acceptable carriers or diluents for therapeutic use are well known in the pharmaceutical art, and are described, for example, in Remington's Pharmaceutical Sciences, Mack Publishing Co. (A.R. Gennaro edit. 1985) hereby incorporated by reference herein. For example, preservatives, stabilizers, dyes and flavoring agents may be provided. Id. at 1449. These include sodium benzoate, sorbic acid and esters of p-hydroxybenzoic acid. In addition, antioxidants and suspending agents may be used.

A pharmaceutically effective dose is that dose required to prevent, inhibit the occurrence, or treat (alleviate a symptom to some extent, preferably all of the symptoms) a disease state. The pharmaceutically effective dose depends on the type of disease, the composition used, the route of administration, the type of mammal being treated, the physical characteristics of the specific mammal under consideration, concurrent medication, and other factors which those skilled in the medical arts will recognize. Generally, an amount between 0.1 mg/kg and 100 mg/kg body weight/day of active ingredients is administered dependent upon potency of the negatively charged polymer.

Alternatively, the enzymatic nucleic acid molecules of the instant invention can be expressed within cells from eukaryotic promoters (e.g., Izant and Weintraub, 1985 Science 229, 345; McGarry and Lindquist, 1986 Proc. Natl. Acad. Sci. USA 83, 399;

Scanlon et al., 1991, Proc. Natl. Acad. Sci USA, 88, 10591-5; Kashani-Sabet et al., 1992 Antisense Res. Dev., 2, 3-15; Dropulic et al., 1992 J. Virol, 66, 1432-41; Weerasinghe et al., 1991 J. Virol, 65, 5531-4; Ojwang et al., 1992 Proc. Natl. Acad. Sci. USA 89, 10802-6; Chen et al, 1992 Nucleic Acids Res., 20, 4581-9; Sarver et al.., 1990 Science 247, 1222-1225; Thompson et al. 1995 Nucleic Acids Res. 23, 2259; Good et al., 1997, Gene Therapy, 4, 45; all of the references are hereby incorporated in their totality by reference herein). Those skilled in the art realize that any nucleic acid can be expressed in eukaryotic cells from the appropriate DNA/RNA vector. The activity of such nucleic acids can be augmented by their release from the primary transcript by a ribozyme (Draper et al., PCT WO 93/23569, and Sullivan et al. PCT WO 94/02595; Ohkawa et al., 1992 Nucleic Acids Symp. Ser., 27, 15-6; Taira et al., 1991, Nucleic Acids Res., 19, 5125-30; Ventura et al., 1993 Nucleic Acids Res., 21, 3249-55; Chowrira et al. 1994 J. Biol. Chem. 269, 25856; all of the references are hereby incorporated in their totality by reference herein).

In another aspect of the invention, enzymatic nucleic acid molecules that cleave target molecules are expressed from transcription units (see for example Couture et al., 25 1996, TIG., 12, 510) inserted into DNA or RNA vectors. The recombinant vectors are preferably DNA plasmids or viral vectors. Ribozyme expressing viral vectors could be constructed based on, but not limited to, adeno-associated virus, retrovirus, adenovirus, or alphavirus. Preferably, the recombinant vectors capable of expressing the ribozymes are delivered as described above, and persist in target cells. Alternatively, viral vectors may be used that provide for transient expression of ribozymes. Such vectors might be repeatedly administered as necessary. Once expressed, the ribozymes cleave the target mRNA. The active ribozyme contains an enzymatic center or core equivalent to those in the examples, and binding arms able to bind target nucleic acid molecules such that cleavage at the target site occurs. Other sequences may be present which do not interfere with such cleavage. Delivery of ribozyme expressing vectors could be systemic, such as by intravenous or intramuscular administration, by administration to target cells ex-planted from the patient followed by reintroduction into the patient, or by any other means that would allow for introduction into the desired target cell (for a review see Couture et al, 1996, TIG., 12, 510).

In one aspect the invention features, an expression vector comprising nucleic acid sequence encoding at least one of the nucleic acid catalyst of the instant invention is disclosed. The nucleic acid sequence encoding the nucleic acid catalyst of the instant invention is operable linked in a manner which allows expression of that nucleic acid molecule.

In another aspect the invention features, the expression vector comprises: a transcription initiation region (e.g., eukaryotic pol I, II or III initiation region); b) a transcription termination region (e.g., eukaryotic pol I, II or III termination region); c) a gene encoding at least one of the nucleic acid catalyst of the instant invention; and wherein said gene is operably linked to said initiation region and said termination region, in a manner which allows expression and/or delivery of said nucleic acid molecule. The vector may optionally include an open reading frame (ORF) for a protein operably linked on the 5′ side or the 3′-side of the gene encoding the nucleic acid catalyst of the invention; and/or an intron (intervening sequences).

Transcription of the ribozyme sequences are driven from a promoter for eukaryotic RNA polymerase I (pol I), RNA polymerase II (pol II), or RNA polymerase III (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters are also used, providing that the prokaryotic RNA polymerase enzyme is expressed in the appropriate cells (Elroy-Stein and Moss, 1990 Proc. Natl. Acad. Sci USA, 87, 6743-7; Gao and Huang 1993 Nucleic Acids Res., 21, 2867-72; Lieber et al., 1993 Methods Enzymol., 217, 47-66; Zhou et al., 1990 Mol. Cell. Biol., 10, 4529-37). Several investigators have demonstrated that ribozymes expressed from such promoters can function in mammalian cells (e.g. Kashani-Sabet et al., 1992 Antisense Res. Dev., 2, 3-15; Ojwang et al., 1992 Proc. Natl. Acad. Sci. USA, 89, 10802-6; Chen etal., 1992 Nucleic Acids Res., 20,4581-9; Yu et al., 1993 Proc. Natl. Acad. Sci. U S A, 90, 6340-4; L'Huillier et al., 1992 EMBO J 11, 4411-8; Lisziewicz et al., 1993 Proc. Natl. Acad. Sci. U. S. A., 90, 8000-4; Thompson et al., 1995 Nucleic Acids Res. 23, 2259; Sullenger & Cech, 1993, Science, 262, 1566). More specifically, transcription units such as the ones derived from genes encoding U6 small nuclear (snRNA), transfer RNA (tRNA) and adenovirus VA RNA are useful in generating high concentrations of desired RNA molecules such as ribozymes in cells (Thompson et al., supra; Couture and Stinchcomb, 1996, supra; Noonberg et al., 1994, Nucleic Acid Res., 22, 2830; Noonberg et al., U.S. Pat. No. 5,624,803; Good et al. 1997, Gene Ther. 4, 45; Beigelman et al., International PCT Publication No. WO 96/18736; all of these publications are incorporated by reference herein. The above ribozyme transcription units can be incorporated into a variety of vectors for introduction into mammalian cells, including but not restricted to, plasmid DNA vectors, viral DNA vectors (such as adenovirus or adeno-associated virus vectors), or viral RNA vectors (such as retroviral or alphavirus vectors) (for a review see Couture and Stinchcomb, 1996, supra).

In yet another aspect the invention features an expression vector comprising nucleic acid sequence encoding at least one of the catalytic nucleic acid molecule of the invention, in a manner which allows expression of that nucleic acid molecule. The expression vector comprises in one embodiment; a) a transcription initiation region; b) a transcription termination region; c) a gene encoding at least one said nucleic acid molecule; and wherein said gene is operably linked to said initiation region and said termination region, in a manner which allows expression and/or delivery of said nucleic acid molecule. In another preferred embodiment the expression vector comprises: a) a transcription initiation region; b) a transcription termination region; c) an open reading frame; d) a gene encoding at least one said nucleic acid molecule, wherein said gene is operably linked to the 3′-end of said open reading frame; and wherein said gene is operably linked to said initiation region, said open reading frame and said termination region, in a manner which allows expression and/or delivery of said nucleic acid molecule. In yet another embodiment the expression vector comprises: a) a transcription initiation region; b) a transcription termination region; c) an intron; d) a gene encoding at least one said nucleic acid molecule; and wherein said gene is operably linked to said initiation region, said intron and said termination region, in a manner which allows expression and/or delivery of said nucleic acid molecule. In another embodiment, the expression vector comprises: a) a transcription initiation region; b) a transcription termination region; c) an intron; d) an open reading frame; e) a gene encoding at least one said nucleic acid molecule, wherein said gene is operably linked to the 3′-end of said open reading frame; and wherein said gene is operably linked to said initiation region, said intron, said open reading frame and said termination region, in a manner which allows expression and/or delivery of said nucleic acid molecule.

Interferons

Type I interferons (IFN) are a class of natural cytokines that includes a family of greater than 25 IFN-oA (Pesta, 1986, Methods Enzymol. 119, 3-14) as well as IFN-β, and IFN-ω). Although evolutionarily derived from the same gene (Diaz et al., 1994, Genomics 22, 540-552), there are many differences in the primary sequence of these molecules, implying an evolutionary divergence in biologic activity. All type I IFN share a common pattern of biologic effects that begin with binding of the IFN to the cell surface receptor (Pfeffer & Strulovici, 1992, Transmembrane secondary messengers for IFN-α/β. In: Interferon. Principles and Medical Applications., S. Baron, D. H. Coopenhaver, F. Dianzani, W. R. Fleischmann Jr., T. K. Hughes Jr., G. R. Kimpel, D. W. Niesel, G. J. Stanton, and S. K. Tyring, eds. 151-160). Binding is followed by activation of tyrosine kinases, including the Janus tyrosine kinases and the STAT proteins, which leads to the production of several IFN-stimulated gene products (Johnson et al., 1994, Sci. Am. 270, 68-75). The IFN-stimulated gene products are responsible for the pleotropic biologic effects of type I IFN, including antiviral, antiproliferative, and immunomodulatory effects, cytokine induction, and HLA class I and class II regulation (Pestka et al., 1987, Annu. Rev. Biochem 56, 727). Examples of IFN-stimulated gene products include 2-5-oligoadenylate synthetase (2-5 OAS), 32-microglobulin, neopterin, p68 kinases, and the Mx protein (Chebath & Revel, 1992, The 2-5 A system: 2-5 A synthetase, isospecies and functions. In: Interferon. Principles and Medical Applications. S. Baron, D. H. Coopenhaver, F. Dianzani, W. R. Jr. Fleischmann, T. K. Jr Hughes, G. R. Kimpel, D. W. Niesel, G. J. Stanton, and S. K. Tyring, eds., pp. 225-236; Samuel, 1992, The RNA-dependent P1/eIF-2α protein kinase. In: Interferon. Principles and Medical Applications. S. Baron, D. H. Coopenhaver, F. Dianzani, W. R. Fleischmann Jr., T. K. Hughes Jr., G. R. Kimpel, D. W. Niesel, G. H. Stanton, and S. K. Tyring, eds. 237-250; Horisberger, 1992, MX protein: function and Mechanism of Action. In: Interferon. Principles and Medical Applications. S. Baron, D. H. Coopenhaver, F. Dianzani, W.R. Fleischmann Jr., T. K. Hughes Jr., G. R. Kimpel, D. W. Niesel, G. H. Stanton, and S. K. Tyring, eds. 215-224). Although all type I IFN have similar biologic effects, not all the activities are shared by each type I IFN, and, in many cases, the extent of activity varies quite substantially for each IFN subtype (Fish et al, 1989, J Interferon Res. 9, 97-114; Ozes et al., 1992, J Interferon Res. 12, 55-59). More specifically, investigations into the properties of different subtypes of IFN-α and molecular hybrids of IFN-α have shown differences in pharmacologic properties (Rubinstein, 1987, J Interferon Res. 7, 545-551). These pharmacologic differences may arise from as few as three amino acid residue changes (Lee et al., 1982, Cancer Res. 42, 1312-1316).

Eighty-five to 166 amino acids are conserved in the known IFN-α subtypes. Excluding the IFN-α pseudogenes, there are approximately 25 known distinct IFN-α subtypes. Pairwise comparisons of these nonallelic subtypes show primary sequence differences ranging from 2% to 23%. In addition to the naturally occurring IFNs, a non-natural recombinant type I interferon known as consensus interferon (CIFN) has been synthesize as a therapeutic compound (Tong et al., 1997, Hepatology 26, 747-754).

Interferon is currently in use for at least 12 different indications including infectious and autoimmune diseases and cancer (Borden, 1992, N. Engl. J Med. 326, 1491-1492). For autoimmune diseases IFN has been utilized for treatment of rheumatoid arthritis, multiple sclerosis, and Crohn's disease. For treatment of cancer IFN has been used alone or in combination with a number of different compounds. Specific types of cancers for which IFN has been used include squamous cell carcinomas, melanomas, hypernephromas, hemangiomas, hairy cell leukemia, and Kaposi's sarcoma. In the treatment of infectious diseases, IFNs increase the phagocytic activity of macrophages and cytotoxicity of lymphocytes and inhibits the propagation of cellular pathogens. Specific indications for which IFN has been used as treatment include: hepatitis B, human papillomavirus types 6 and 11 (i.e. genital warts) (Leventhal et al., 1991, N Engl J Med 325, 613-617), chronic granulomatous disease, and hepatitis C virus.

Numerous well controlled clinical trials using IFN-α alpha in the treatment of chronic HCV infection have demonstrated that treatment three times a week results in lowering of serum ALT values in approximately 50% (range 40% to 70%) of patients by the end of 6 months of therapy (Davis et al., 1989, The new England Journal of Medicine 321, 1501-1506; Marcellin et al., 1991, Hepatology 13, 393-397; Tong et al., 1997, Hepatology 26, 747-754; Tong et al., Hepatology 26, 1640-1645). However, following cessation of interferon treatment, approximately 50% of the responding patients relapsed, resulting in a “durable” response rate as assessed by normalization of serum ALT concentrations of approxyimately 20 to 25%. In addition, studies that have examined six months of type 1 interferon therapy using changes in HCV RNA values as a clinical endpoint have demonstrated that up to 35% of patients will have a loss of HCV RNA by the end of therapy (Tong et al., 1997, supra). However, as with the ALT endpoint, about 50% of the patients relapse six months following cessation of therapy resulting in a durable virologic response of only 12% (23). Studies that have examined 48 weeks of therapy have demonstrated that the sustained virological response is up to 25%

Ribozymes in combination with IFN have the potential to improve the effectiveness of treatment of HCV or any of the other indications discussed above. Ribozymes targeting RNAs associated with diseases such as infectious diseases, autoimmune disases, and cancer, can be used individually or in combination with other therapies such as IFN to achieve enhanced efficacy.

### EXAMPLES

The following are non-limiting examples showing the selection, isolation, synthesis and activity of enzymatic nucleic acids of the instant invention.

The following examples demonstrate the selection of ribozymes that cleave HCV RNA. The methods described herein represent a scheme by which ribozymes may be derived that cleave other RNA targets required for HCV replication.

### Example 1

### Identification of Potential Ribozyme Cleavage Sites in HCV RNA

The sequence of HCV RNA was screened for accessible sites using a computer folding algorithm. Regions of the MRNA that did not form secondary folding structures and contained potential hammerhead and/or hairpin ribozyme cleavage sites were identified. The sequences of these cleavage sites are shown in tables IV-VIII.

### Example 2

### Selection of Ribozyme Cleavage Sites in HCV RNA

To test whether the sites predicted by the computer-based RNA folding algorithm corresponded to accessible sites in HCV RNA, 20 hammerhead sites were selected for analysis. Ribozyme target sites were chosen by analyzing genomic sequences of HCV (Input Sequence=HPCJTA (Acc#D11168 & D01171)) and prioritizing the sites on the basis of folding. Hammerhead ribozymes were designed that could bind each target (see FIG. 1) and were individually analyzed by computer folding (Christoffersen et al. 1994 J Mol Struc. Theochem, 311, 273; Jaeger et al., 1989, Proc. Natl. Acad. Sci. USA, 86, 7706) to assess whether the ribozyme sequences fold into the appropriate secondary structure. Those ribozymes with unfavorable intramolecular interactions between the binding arms and the catalytic core were eliminated from consideration. As noted below, varying binding arm lengths can be chosen to optimize activity. Generally, at least 5 bases on each arm are able to bind to, or otherwise interact with, the target RNA.

Selection of ribozyme candidates was initiated by scanning for all hammerhead cleavage sites in an HCV RNA sequence derived from a patient infected with HCV genotype 1b. The results of this sequence analysis are shown in Table III. As seen by Table III, 1300 hammerhead ribozyme sites were identified by this analysis. Next, in order to identify hammerhead ribozyme candidates that would cleave in the conserved regions of the HCV genome, a sequence alignment of approximately 50 HCV isolates from genotypes 1a, 1b, 2a, 2b, 2c, 3a, 3b, 4a, 5a, and 6 was completed. Within genotype sites were identified that are in areas having the greatest sequence identity between all isolates examined. This analysis reduced the hammerhead ribozyme candidates to about 23 (Table III).

Due to the high sequence variability of the HCV genome, selection of ribozymes for broad therapeutic applications should probably involve the conserved regions of the HCV genome. A list of the thirty-hamnmerhead ribozymes derived from the conserved regions (5′- Non-Coding Region (NCR), 5′- end of core protein coding region, and 3′-NCR) of the HCV genome is shown in Table IV. In general, ribozymes targeted to sites located in the 5′ terminal region of the HCV genome should block translation while ribozymes cleavage sites located in the 3′ terminal region of the genome should block RNA replication.

### Example 3

### Chemical Synthesis and Purification of Ribozymes

Ribozymes of the hammerhead or hairpin motif were designed to anneal to various sites in the RNA message. The binding arms are complementary to the target site sequences described above. The ribozymes were chemically synthesized. The method of synthesis used followed the procedure for normal RNA synthesis as described in Usman et al., (1987 J. Am. Chem. Soc., 109, 7845), Scaringe et al., (1990 Nucleic Acids Res., 18, 5433) and Wincott et al., supra, and made use of common nucleic acid protecting and coupling groups, such as dimethoxytrityl at the 5′-end, and phosphoramidites at the 3′-end. The average stepwise coupling yields were>98%.

Inactive hammerhead ribozymes were synthesized by substituting switching the order of G₅A₆ and substituting a U for A₁₄ (numbering from Hertel et al., 1992 Nucleic Acids Res., 20, 3252). Hairpin ribozymes were synthesized in two parts and annealed to reconstruct the active ribozyme (Chowrira and Burke, 1992 Nucleic Acids Res., 20, 2835-2840). Ribozymes were also synthesized from DNA templates using bacteriophage T7 RNA polymerase (Milligan and Uhlenbeck, 1989, Methods Enzymol. 180, 51). Ribozymes were modified to enhance stability by modification with nuclease resistant groups, for example, 2′-amino, 2′-C-allyl, 2′-flouro, 2′-O-methyl, 2′-H (for a review see Usman and Cedergren, 1992 TIBS 17, 34). Ribozymes were purified by gel electrophoresis using general methods or were purified by high pressure liquid chromatography (HPLC; See Wincott et al., supra; the totality of which is hereby incorporated herein by reference) and were resuspended in water. The sequences of the chemically synthesized ribozymes used in this study are shown below in Table IV -IX.

### Example 4

### Ribozyme Cleavage of HCV RNA Target in vitro

Ribozymes targeted to the HCV are designed and synthesized as described above. These ribozymes can be tested for cleavage activity in vitro, for example using the following procedure. The target sequences and the nucleotide location within the HCV are given in Table IV.

Cleavage Reactions: Full-length or partially full-length, internally-labeled target RNA for ribozyme cleavage assay is prepared by in vitro transcription in the presence of [α⁻³²p] CTP, passed over a G 50 Sephadex column by spin chromatography and used as substrate RNA without further purification. Alternately, substrates are 5′-³²P-end labeled using T4 polynucleotide kinase enzyme. Assays are performed by pre-warming a 2X concentration of purified ribozyme in ribozyme cleavage buffer (50 mM Tris-HCl, pH 7.5 at 37° C., 10 mM MgCl₂) and the cleavage reaction was initiated by adding the 2X ribozyme mix to an equal volume of substrate RNA (maximum of 1-5 nM) that was also pre-warmed in cleavage buffer. As an initial screen, assays are carried out for 1 hour at 37° C. using a final concentration of either 40 nM or 1 mM ribozyme, i.e., ribozyme excess. The reaction is quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

### Example 5

### Ability of HCV Ribozymes to Cleave HCV RNA in Patient Serum

Ribozymes targeting sites in HCV RNA were synthesized using modifications that confer nuclease resistance (Beigelman, 1995, J. Biol. Chem. 270, 25702). It has been well documented that serum from chronic hepatitis C patients contains on average 3×10⁶ copies/ml of HCV RNA. To further select ribozyme product candidates, the 30 HCV specific ribozymes are characterized for HCV RNA cleavage activity utilizing HCV RNA isolated from the serum of genotype 1b HCV patients. The best candidates from the HCV genotype1b screen will be screened against isolates from the wide range of HCV genotypes including 1a, 1b, 2a, 2b, 2c, 3a, 3b, 4a, 5a, and 6. Therefore, it is possible to select ribozyme candidates for further development based on their ability to broadly cleave HCV RNA from a diverse range of HCV genotypes and quasispecies.

### Example 6

### Ribozyme Cleavage of Conserved HCV RNA Target Sites in vitro

There are three regions of the genome that are highly conserved, both within a genotype and across different genotypes. These conserved sequences occur in the 5′ and 3′ non-coding regions (NCRs) as well as the 5′-end of the Core Protein coding region. These regions are thought to be important for HCV RNA replication and translation. Thus, therapeutic agents that target these conserved HCV genornic regions may have a significant impact over a wide range of HCV genotypes. The presence of quasispecies, and the potential for infection with more than one genotype makes this a critical feature of an effective therapy. Moreover, it is unlikely that drug resistance will occur, since mutations that have been suggested to lead to drug resistance typically do not occur within these highly conserved regions. In order to target multiple genotypes and decrease the chance of developing drug resistance, Applicant has designed ribozymes that cleave in regions of identity within the conserved regions discussed above.

Sequence alignments were performed for the 5′ NCR, the 5′ end of the Core Protein coding region, and the 3′ NCR. For the 5′ NCR, 34 different isolates representing genotypes 1a, 1b, 2a, 2b, 2c, 3a, 3b, 4a, 4f, and 5a were aligned. The alignments included the sequences from nucleotide position 1 to nucleotide position 350 (18 nucleotides downstream of the initiator ATG codon), using the reported sequence “HPCKlSI” as the reference for numbering. For the Core Protein coding region, 44 different isolates representing genotypes 1a, 1b, 2a, 2b, 2c, 3a, 3b, 4a, 4c, 4f, 5a, and 6a were aligned. These alignments included 600 nucleotides, beginning 8 nucleotides upstream of the initiator ATG codon. As the reference for numbering, the reported sequence “HPCCOPR” was used, with the “C” eight nucleotides upstream of the initiator codon ATG designated as “I”. For the 3′ NCR region, 20 different isolates representing genotypes1b, 2a, 2b, 3a, and 3b were aligned. These alignments included sequences in the 3′ terminal 235 nucleotides of the genome, with the reported sequence “D855 16” used as the reference for numbering, and the 235h nucleotide from the 3′ end designated as “1”.

During analysis of the alignments of each region, each sequence was compared to the respective reference sequence (identified above), and regions of identity across all isolates were determined. All potential ribozyme sites were identified in the reference sequence. The highest priority for choosing ribozyme sites was that the site should have 100% identity across all isolates aligned, at every position in both the cleavage site and binding arms. Ribozyme sites that met these criteria were chosen. In addition, two specific allowances were made as follows. 1) If a potential ribozyme site had 100% sequence identity at all except one or two nucleotide positions, then the actual nucleotide at that position was examined in the isolate(s) that differed. If that nucleotide was such that a ribozyme designed to allow “G:U wobble” base-paring could function on all the isolates, then that site was chosen. 2) If a potential ribozyme site had 100% sequence identity at all except one or two nucleotide positions, then the genotype of the isolate which contained the differing nucleotide(s) was examined. If the genotype of the isolate that differed was of extremely rare prevalence, then that site was also chosen.

Ribozyme sites identified and referred to below use the following nomenclature: “region of the genome in which the site exists” followed by “nucleotide position 5′ to the cleavage site” (according to the reference sequence and numbering described above). For example, a ribozyme cleavage site at nucleotide position 67 in the 5′ NCR is designated “5-67”, and a ribozyme cleavage site at position 48 in the core coding region is designated “c48”.

A number of these ribozymes were screened in an in vitro HCV cleavage assay to select appropriate ribozyme candidates for cell culture studies. The ribozymes selected for screening targeted the 5′ UTR region that is necessary for HCV translation. These sites are all conserved among the 8 major HCV genotypes and 18 subtypes, and have a high degree of homology in every HCV isolate that was used in the analysis described above. HCV RNA of four different genotypes (1b, 2a, 4, and 5) were isolated from human patients and the 5′ HCV UTR and 5′ core region were amplified using RT-PCR. Run-off transcripts of the 5′ HCV UTR region (-750 nt transcripts) were prepared from the RT-PCR products, which contained a T7 promoter, using the T7 Megascript transcription kit and the manufacturers protocol (Ambion, Inc.). Unincorporated nucleotides are removed by spin column filtration on Bio-Gel P-60 resin (Bio-Rad). The filtered transcript was 5′ end labeled with ³²p using Polynucleotide Kinase (Boehringer/Mannheim) and 150μCi/μl Gamma-32P-ATP (NEN) using the enzyme manufacturer's protocol. The kinased transcript is spin purified again to remove unincorporated Gamma-32P-ATP and gel purified on 5% polyacrylamide gel.

Ribozymes targeting various sites from table IV were selected and tested on the 5′ HCV UTR transcript sequence to test the efficiency of RNA cleavage. 15 ribozymes were synthesized as previously described (Wincott et al., supra).

Assays were performed by pre-warming a 2X (2 μM ) concentration of purified ribozyme in ribozyme cleavage buffer (50 mM TRIS pH 7.5, 10 M MgCI₂, 10 units RNase Inhibitor (Boehringer/Mannheim), 10 mM DTT, 0.5 μg tRNA) and the cleavage reaction was initiated by adding the 2X ribozyme mix to an equal volume of substrate RNA (17.46 pmole final concentration) that was also pre-warmed in cleavage buffer. The assay was carried out for 24 hours at 37° C. using a fmal concentration of 1 μM ribozyme, iLe., ribozyme excess. The reaction was quenched by the addition of an equal volume of 95% formamide, 20 mM EDTA, 0.05% bromophenol blue and 0.05% xylene cyanol after which the sample is heated to 95° C. for 2 minutes, quick chilled and loaded onto a denaturing polyacrylamide gel. Substrate RNA and the specific RNA cleavage products generated by ribozyme cleavage are visualized on an autoradiograph of the gel. The percentage of cleavage is determined by Phosphor Imager® quantitation of bands representing the intact substrate and the cleavage products.

Observed cleavage fragment sizes from the gels are correlated to predicted fragment sizes by comparison to the RNA marker. The optical density of expected cleavage fragments are determined from the phosphorimage plates and ranked from highest density, indicating the most cleavage product, to lowest of each genotype of HCV transcript tested. The top 3 cleaving ribozymes (out of 15 ribozymes tested) are given ranking values of 5, the next 3 highest densities are given ranking values of 4, etc for every genotype tested. The ranking values for each ribozyme are averaged between the genotypes tested. Individual and average ribozyme ranking values are graphed and compared. The results (FIG. 2) demonstrate that many of these tested ribozymes are able to to give high levels of cleavage regardless of genotype. In particular, ribozymes targeting site HCV.5-258, HCV.5-294, HCV.5-313 (Sakamoto et al., J. Clinical Investigation 1996 98(12):2720-2728), and HCV.5-318 (table IV) appear to demonstrate a consistent pattern of RNA cleavage

### Example 7

### Inhibition of Luciferase Activity Using HCV Tarizeting Ribozymes in OST7 Cells

The capability of ribozymes to inhibit HCV RNA intracellularly was tested using a dual reporter system that utilizes both firefly and Renilla luciferase (FIG. 3). The ribozymes targeted to the 5′ HCV UTR region, which when cleaved, would prevent the translation of the transcript into luciferase. OST-7 cells were plated at 12,500 cells per well in black walled 96 well plates (Packard) in medium DMEM containing 10% fetal bovine serum, 1% pen/strep, and 1% L-glutamine and incubated at 37° C. overnight. A plasmid containing T7 promoter expressing 5′ HCV UTR and firefly luciferase (T7Cl-341 (Wang etal., 1993, J. of Virol. 67, 3338-3344)) was mixed with a pRLSV40 Renilla control plasmid (Promega Corporation) followed by ribozyme, and cationic lipid to make a 5X concentration of the reagents (T7C1-341 (4 gg/ml), pRLSV40 renilla luciferase control (6 μg/ml), ribozyme (250 nM), transfection reagent (28.5 μg/ml).

The complex mixture was incubated at 37° C. for 20 minutes. The media was removed from the cells and 120 μl of Opti-mem media was added to the well followed by 30 μt of the 5X complex mixture. 150 μl of Opti-mem was added to the wells holding the untreated cells. The complex mixture was incubated on OST-7 cells for 4 hours, lysed with passive lysis buffer (Promega Corporation) and luminescent signals were quantified using the Dual Luciferase Assay Kit using the manufacturer's protocol (Promega Corporation). The ribozyme sequences used are given in table IV. The ribozymes used were of the hammerhead motif. The hammerhead ribozymes were chemically modified such that the ribozyme consists of ribose residues at five positions (see for example FIG. 7); position 4 has either 2′-C-allyl or 2′-amino modification; position 7 has either 2′-amnino modification or 2-O-methyl modification; the remaining nucleotide positions contain 2′-O-methyl substitutions; four nucleotides at the 5′ terminus contains phosphorothioate substitutions. Additionally, the 3′ end of the ribozyme includes a 3′-3′ linked inverted abasic moiety (abasic deoxyribose; iH). The data (FIG. 4) is given as a ratio between the firefly and Renilla luciferase fluorescence. All of the ribozymes targeting 5′ HCV UTR were able to reduce firefly luciferase signal relative to renilla luciferase.

### Example 9

### Ribozyme Mediated Inhibition of Luciferase Activity Compared to its Inactive Control in OST-7 Cells

The dual reporter system described above was utilized to determine the level of reduction of luciferase activity mediated by a ribozyme compared to its inactive control. Ribozymes, having the chemical composition described in the previous example, to sites HCV 313 and 318 (table IV) and their inactive controls were synthesized as above. The inactive control has the same nucleotide base composition as the active ribozyme but the nucleotide sequence has been scrambled. The protocols utilized for tissue culture and the luciferase assay was exactly as given in example 8 except the ribozyme concentration in the 5X complex mixture was 1 mM (final concentration on the cells was 200 nM).

The results are given in FIG. 5. The ribozyme targeting HCV.5-3 18 was able to greatly reduce firefly luciferase activity compared to the untreated and inactive controls. The ribozyme targeting HCV.5-313 was able to slightly reduce firefly luciferase activity compared to the inactive control.

### Example 10:

### Ribozyme Inhibition of Viral Replication

During HCV infection, viral RNA is present as a potential target for ribozyme cleavage at several processes: uncoating, translation, RNA replication and packaging. Target RNA may be more or less accessible to ribozyme cleavage at any one of these steps. Although the association between the HCV initial ribosome entry site (IRES) and the translation apparatus is mimicked in the HCV 5′ UTR/luciferase reporter system (example 9), these other viral processes are not represented in the OST7 system. The resulting RNA/protein complexes associated with the target viral RNA are also absent. Moreover, these processes may be coupled in an HCV-infected cell which could further impact target RNA accessibility. Therefore, we tested whether ribozymes designed to cleave the HCV 5′ UTR could effect a replicating viral system.

Recently, Lu and Wimmer characterized an HCV-poliovirus chimera in which the poliovirus IRES was replaced by the IRES from HCV (Lu & Wimmer, 1996, Proc. Natl. Acad. Sci. USA. 93, 1412-1417). Poliovirus (PV) is a positive strand RNA virus like HCV, but unlike HCV is non-enveloped and replicates efficiently in cell culture. The HCV-PV chimera expresses a stable, small plaque phenotype relative to wild type PV.

The following ribozymes were synthesized for the experiment (table VIII): ribozyme targeting site 183 (3 5′-end phosphorothioate linkages), scrambled control to site 183, ribozyme to site 318 (3 5′-end phosphorothioate linkages), ribozyme targeting site 183 (4 5′-end phosphorothioate linkages), inactive ribozyme targeting site 183 (4 5′-end phosphorothioate linkages). HeLa cells were infected with the HCV-PV chimera for 30 minutes and immediately treated with ribozyme. HeLa cells were seeded in U-bottom 96-well plates at a density of 9000-10,000 cells/well and incubated at 37° C. under 5% CO₂ for 24 h. Transfection of ribozyme (200 nM) was achieved by mixing of 10X ribozyme (2000 nM) and 10X of a cationic lipid (80μpg/ml) in DMEM (Gibco BRL) with 5% fetal bovine serum (FBS). Ribozyme/lipid complexes were allowed to incubate for 15 minutes at 37° C. under 5% CO₂. Medium was aspirated from cells and replaced with 80 μs of DMEM (Gibco BRL) with 5% FBS serum, followed by the addition of 20 μls of 10X complexes. Cells were incubated with complexes for 24 hours at 37° C. under 5% CO₂.

The yield of HCV-PV from treated cells (FIG. 6A) was quantified by plaque assay. The plaque assays were performed by diluting virus samples in serum-free DMEM (Gibco BRL) and applying 100 μl to HeLa cell monolayers (˜80% confluent) in 6-well plates for 30 minutes. Infected monolayers were overlayed with 3 ml 1.2% agar (Sigma) and incubated at 37° C. under 5% CO₂. Two-three days later the overlay was removed, monolayers were stained with 1.2% crystal violet, and plaque forming units were counted. The data is shown in FIG. 6A. Ribozymes to site 183 inhibited HCV-PV replication by>80% (P<0.05) compared to the scrambled control (FIG. 6A, first two bars). In addition, 3 or 4 phosphorothioate stabilization was equally effective (P<0.05 vs. control for each) in inhibiting viral replication (compare 1st and 4th bar in FIG. 6A). Ribozymes to the 318 site also had a statistically significant (P<0.05), effect on viral replication (compare ₂nd and ₃rd bar in FIG. 6A).

To confirm that a ribozyme cleavage mechanism was responsible for the inhibition of HCV-PV replication observed, HCV-PV infected cells were treated with ribozymes to site 183 that maintained binding arm sequences but contained a mutation in the catalytic core to attenuate cleavage activity (Table I). Viral replication in these cells was not inhibited compared to cells treated with the scrambled control ribozyme (FIG. 6A, ₄th and ₅th bar), indicating that ribozyme cleavage activity was required for the inhibition of HCV-PV replication observed. In addition, ribozymes targeting site 183 of the HCV 5′ UTR had no effect on wild type PV replication (FIG. 6B). These data provide evidence that the ribozyme-mediated inhibition of HCV-PV replication was dependent upon the HCV 5′ UTR and not a general inhibition of PV replication.

Ribozymes to site 183 were also tested for the ability to inhibit HCV-PV replication during a single infectious cycle in HeLa cells (FIG. 8). Cells treated with ribozyme to site 183 (7/4 format) produced significantly less virus than cells treated with the scrambled control (>80% inhibition at 8h post infection, P <0.001).

### Example 11

### Shortening of Ribozyme Lengths

All the ribozymes described in example I0 above contained 7 nucleotides on each binding arms and contained a 4 base-paired stem II element (7/4 format). For pharmaceutical manufacture of a therapeutic ribozyme it is advantageous to minimize sequence length if possible. Thus ribozymes to site 183 were shortened by removing the outer most nucleotide from each binding arm such that the ribozyme has six nucleotides in each binding arm and the stem II region is four base-paired long (6/4 format); removing one base-pair (2 nucleotides) in stem II resulting in a 3 base-paired stem II (7/3 format); or removing one nucleotide from each binding arm and shortening the stem II by one base-pair (6/3 format) (See FIG. 7 for a schematic representation of each of these ribozymes. Ribozymes in all tested formats gave significant inhibition of viral replication (FIG. 8) with the 7/4, 7/3 and 6/3 formats being almost identical at the 8h timepoint (P<0.001 across time course for all formats). The shortest ribozyme tested (6/3 format) was slightly more efficacious (>90% inhibition, P<0.001) than the 7/4 ribozyme (˜80% inhibition, P<0.001). The 6/3 ribozyme may have a greater ability to access site 183 in the HCV-PV chimera.

### Example 12

### Combination Therapy of HCV Ribozymes and Interferon

HeLa cells (10,000 cells per well) were pre-treated with 12.5 Units/ml of Interferon alpha in complete media (DMEM+5% FBS) or pre-treated with complete media alone for 4 hours and then infected with HCV-PV at an MOI=0.1 for 30 minutes. The viral inoculum was then removed and 200 nM ribozyme targeted to HCV site 183 (Rz) or binding attenuated control, which has mutations in the catalytic core of the ribozyme that severely attenuates the activity of the ribozyme, (BAC) was delivered using cationic lipid in complete media for 24 hours. After 24 hours, the cells were lysed three times by freeze/thaw to release virus and virus was quantified by plaque assay. Viral yield is shown as mean plaque forming units per ml (pfu/ml)+SEM. The data is shown in FIG. 10.

Pre-treatment with interferon (IFN) reduces the viral yield by ˜10⁻¹ in control treated cells (BAC+IFN versus BAC). Ribozyme treated cells produce 2×10⁻¹ less virus than control-treated cells (Rz versus BAC). The combination of Rz and IFN treatment results in a synergistic 4×10⁻² reduction in viral yield (Rz+IFN versus BAC). An additive effect would result in only a 3×10⁻¹ reduction (1×10^{-1+2×10}⁻¹).

### Example 13

### Inhibition of Hepatitis C virus Usingz other Ribozyme Motifs

A number of varying ribozyme motifs (RPI motifs 1-3; FIG. 9), were tested for their ability to inhibit HCV propagation in tissue culture. An example of RPI motif I is described in Kore et al., 1998, Nucleic Acids Research 26, 4116-4120, while an example of RPI motif II is described in Ludwig & Sproat, International PCT Publication No. WO 98/58058). RPI motif III is a new ribozyme motif which applicant has recently developed and an example of this motif was tested herein.

OST7 cells were maintained in Dulbecco's modified Eagle's medium (GIBCO BRL) supplemented with 10% fetal calf serum, L-glutamine (2 mM) and penicillin/streptomycin. For transfections, OST7 cells were seeded in black-walled 96-well plates (Packard Instruments) at a density of 12,500 cells/well and incubated at 37° C. under 5% CO₂ for 24 hours. Co-transfection of target reporter HCVT7C (0.8 μg/ml), control reporter pRLSV40, (1.2 μg/ml) and ribozyme, 50-200 nM was achieved by the following method: a 5X mixture of HCVT7C (4 μg/ml), pRLSV40 (6 μg/ml), ribozyme (250-1000 nM) and cationic lipid (28.5 μg/ml) was made in 150 μls of OPTI-MEM (GIBCO BRL) minus serum. Reporter/ribozyme/lipid complexes were allowed to form for 20 minutes at 37° C. under 5% CO₂. Medium was aspirated from OST7 cells and replaced with 120 tls of OPTI-MEM (GIBCO BRL) minus serum, immediately followed by the addition of 30 μs of 5X reporter/ribozyme/lipid complexes. Cells were incubated with complexes for 4 hours at 37° C. under 5% CO₂. Luciferase assay was performed as described in example 7. The data is summarized in table IX, with each motif's results listed along with its control. All of the ribozyme motifs were able to reduce the amount of HCV produced by the cells compared to the irrelevant controls.

### CELL CULTURE ASSAYS

Although there have been reports of replication of HCV in cell culture (see below), these systems are difficult to replicate and have proven unreliable. Therefore, as was the case for development of other anti-HCV therapeutics such as interferon and ribavirin, after demonstration of safety in animal studies applicant can proceed directly into a clinical feasibility study.

Several recent reports have documented in vitro growth of HCV in human cell lines (Mizutani et al., Biochem Biophys Res Commun 1996 227(3):822-826; Tagawa et al., Journal of Gasteroenterology and Hepatology 1995 10(5):523-527; Cribier et al., Journal of General Virology 76(10):2485-2491; Seipp et al., Journal of General Virology 1997 78(10)2467-2478; Iacovacci et al., Research Virology 1997 148(2):147-151; Iocavacci et al., Hepatology 1997 26(5) 1328-1337; Ito et al., Journal of General Virology 1996 77(5):1043-1054; Nakajima et al., Journal of Virology 1996 70(5):3325-3329; Mizutani et al., Journal of Virology 1996 70(10):7219-7223; Valli et al., Res Virol 1995 146(4): 285-288; Kato et al., Biochem Biophys Res Comm 1995 206(3):863-869). Replication of HCV has been demonstrated in both T and B cell lines as well as cell lines derived from human hepatocytes. Demonstration of replication was documented using either RT-PCR based assays or the b-DNA assay. It is important to note that the most recent publications regarding HCV cell cultures document replication for up to 6-months.

In addition to cell lines that can be infected with HCV, several groups have reported the successful transformation of cell lines with cDNA clones of full-length or partial HCV genomes (Harada et al., Journal of General Virology 1995 76(5)1215-1221; Haramatsu et al., Journal of Viral Hepatitis 1997 4S(l):61-67; Dash et al., American Journal of Pathology 1997 151(2):363-373; Mizuno et al., Gasteroenterology 1995 109(6):1933-40; Yoo et al., Journal Of Virology 1995 69(l):32-38).

### ANIMAL MODELS

The best characterized animal system for HCV infection is the chimpanzee.

Moreover, the chronic hepatitis that results from HCV infection in chimpanzees and humans is very similar. Although clinically relevant, the chimpanzee model suffers from several practical impediments that make use of this model difficult. These include; high cost, long incubation requirements and lack of sufficient quantities of animals. Due to these factors, a number of groups have attempted to develop rodent models of chronic hepatitis C infection. While direct infection has not been possible several groups have reported on the stable transfection of either portions or entire HCV genomes into rodents (Yamamoto et al., Hepatology 1995 22(3): 847-855; Galun et al., Journal of Infectious Disease 1995 172(1):25-30; Koike et al, Journal of general Virology 1995 76(12)3031-3038; Pasquinelli et al, Hepatology 1997 25(3): 719-727; Hayashi et al, Princess Takamatsu Symp 1995 25:1430149; Mariya K, Yotsuyanagi H, Shintani Y, Fujie H, Ishibashi K, Matsuura Y, Miyamura T, Koike K. Hepatitis C virus core protein induces hepatic steatosis in transgenic mice. Journal of General Virology 1997 78(7) 1527-1531; Takehara et al, Hepatology 1995 21(3):746-751; Kawamura et al, Hepatology 1997 25(4): 1014-1021). In addition, transplantation of HCV infected human liver into immunocompromised mice results in prolonged detection of HCV RNA in the animal's blood.

Diagnostic uses

Ribozymes of this invention may be used as diagnostic tools to examine genetic drift and mutations within diseased cells or to detect the presence of HCV RNA in a cell. The close relationship between ribozyme activity and the structure of the target RNA allows the detection of mutations in any region of the molecule, which alters the base-pairing and three-dimensional structure of the target RNA. By using multiple ribozymes described in this invention, one may map nucleotide changes, which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defmed as important mediators of the disease. These experiments will lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes of this invention are well known in the art, and include detection of the presence of mRNAs associated with HCV related condition. Such RNA is detected by determining the presence of a cleavage product after treatment with a ribozyme using standard methodology.

In a specific example, ribozymes which can cleave only wild-type or mutant forms of the target RNA are used for the assay. The first ribozyme is used to identify wild-type RNA present in the sample and the second ribozyme will be used to identify mutant RNA in the sample. As reaction controls, synthetic substrates of both wild-type and mutant RNA will be cleaved by both ribozymes to demonstrate the relative ribozyme efficiencies in the reactions and the absence of cleavage of the “non-targeted” RNA species. The cleavage products from the synthetic substrates will also serve to generate size markers for the analysis of wild-type and mutant RNAs in the sample population. Thus each analysis will require two ribozymes, two substrates and one unknown sample which will be combined into six reactions. The presence of cleavage products will be determined using an RNase protection assay so that full-length and cleavage fragments of each RNA can be analyzed in one lane of a polyacrylamide gel. It is not absolutely required to quantify the results to gain insight into the expression of mutant RNAs and putative risk of the desired phenotypic changes in target cells. The expression of MRNA whose protein product is implicated in the development of the phenotype (i.e., HCV) is adequate to establish risk. If probes of comparable specific activity are used for both transcripts, then a qualitative comparison of RNA levels will be adequate and will decrease the cost of the initial diagnosis. Higher mutant form to wild-type ratios will be correlated with higher risk whether RNA levels are compared qualitatively or quantitatively.

Additional Uses

Potential usefulness of sequence-specific enzymatic nucleic acid molecules of the instant invention might have many of the same applications for the study of RNA that DNA restriction endonucleases have for the study of DNA (Nathans et al., 1975 Ann. Rev. Biochem. 44:273). For example, the pattern of restriction fragments could be used to establish sequence relationships between two related RNAs, and large RNAs could be specifically cleaved to fragments of a size more useful for study. The ability to engineer sequence specificity of the ribozyme is ideal for cleavage of RNAs of unknown sequence.

Other embodiments are within the following claims.

TABLE I
Characteristics of naturally occurring ribozymes

Group I Introns
Size: ˜150 to >1000 nucleotides.
Requires a U in the target sequence immediately 5′ of the cleavage site.
Binds 4-6 nucleotides at the 5′-side of the cleavage site.
Reaction mechanism: attack by the 3′-OH of guanosine to generate cleavage products with
3′-OH and 5′-guanosine.
Additional protein cofactors required in some cases to help folding and maintainance of
the active structure.
Over 300 known members of this class. Found as an intervening sequence in Tetrahymena
thermophila rRNA, fungal mitochondria, chloroplasts, phage T4, blue-green algae, and
others.
Major structural features largely established through phylogenetic comparisons,
mutagenesis, and biochemical studies [ⁱ,ⁱⁱ].
Complete kinetic framework established for one ribozyme [ⁱⁱⁱ,^{iv},^{v},^{vi}].
Studies of ribozyme folding and substrate docking underway [^{vii},^{viii},^{ix}].
Chemical modification investigation of important residues well established [^{x},^{xi}].
The small (4-6 nt) binding site may make this ribozyme too non-specific for targeted RNA
cleavage, however, the Tetrahymena group I intron has been used to repair a “defective”
β-galactosidase message by the ligation of new β-galactosidase sequences onto the
defective message [^{xii}].
RNAse P RNA (M1 RNA)
Size: ˜290 to 400 nucleotides.
RNA portion of a ubiquitous ribonucleoprotein enzyme.
Cleaves tRNA precursors to form mature tRNA [^{xiii}].
Reaction mechanism: possible attack by M²⁺-OH to generate cleavage products with 3′-
OH and 5′-phosphate.
RNAse P is found throughout the prokaryotes and eukaryotes. The RNA subunit has
been sequenced from bacteria, yeast, rodents, and primates.
Recruitment of endogenous RNAse P for therapeutic applications is possible through
hybridization of an External Guide Sequence (EGS) to the target RNA [^{xiv},^{xv}]
Important phosphate and 2′ OH contacts recently identified [^{xvi},^{xvii}]
Group II Introns
Size: >1000 nucleotides.
Trans cleavage of target RNAs recently demonstrated [^{xviii},^{xix}].
Sequence requirements not fully determined.
Reaction mechanism: 2′-OH of an internal adenosine generates cleavage products with 3′-
OH and a “lariat” RNA containing a 3′-5′ and a 2′-5′ branch point.
Only natural ribozyme with demonstrated participation in DNA cleavage [^{xx},^{xxi}] in
addition to RNA cleavage and ligation.
Major structural features largely established through phylogenetic comparisons [^{xxii}].
Important 2′ OH contacts beginning to be identified [^{xxiii}]
Kinetic framework under development [^{xxiv}]
Neurospora VS RNA
Size: ˜144 nucleotides.
Trans cleavage of hairpin target RNAs recently demonstrated [^{xxv}].
Sequence requirements not fully determined.
Reaction mechanism: attack by 2′-OH 5′ to the scissile bond to generate cleavage products
with 2′,3′-cyclic phosphate and 5′-OH ends.
Binding sites and structural requirements not fully determined.
Only 1 known member of this class. Found in Neurospora VS RNA.
Hammerhead Ribozyme
(see text for references)
Size: ˜13 to 40 nucleotides.
Requires the target sequence UH immediately 5′ of the cleavage site.
Binds a variable number nucleotides on both sides of the cleavage site.
Reaction mechanism: attack by 2′-OH 5′ to the scissile bond to generate cleavage products
with 2′,3′-cyclic phosphate and 5′-OH ends.
14 known members of this class. Found in a number of plant pathogens (virusoids) that
use RNA as the infectious agent.
Essential structural features largely defined, including 2 crystal structures [^{xxvi},^{xxvii}]
Minimal ligation activity demonstrated (for engineering through in vitro selection) [^{xxviii}]
Complete kinetic framework established for two or more ribozymes [^{xxix}].
Chemical modification investigation of important residues well established [^{xxx}].
Hairpin Ribozyme
Size: ˜50 nucleotides.
Requires the target sequence GUC immediately 3′ of the cleavage site.
Binds 4-6 nucleotides at the 5′-side of the cleavage site and a variable number to the 3′-
side of the cleavage site.
Reaction mechanism: attack by 2′-OH 5′ to the scissile bond to generate cleavage products
with 2′,3′-cyclic phosphate and 5′-OH ends.
3 known members of this class. Found in three plant pathogen (satellite RNAs of the
tobacco ringspot virus, arabis mosaic virus and chicory yellow mottle virus) which uses
RNA as the infectious agent.
Essential structural features largely defined [^{xxxi},^{xxxii},^{xxxiii},^{xxiv}]
Ligation activity (in addition to cleavage activity) makes ribozyme amenable to
engineering through in vitro selection [^{xxxv}]
Complete kinetic framework established for one ribozyme [^{xxxvi}].
Chemical modification investigation of important residues begun [^{xxxvii},^{xxxviii}].
Hepatitis Delta Virus (HDV) Ribozyme
Size: ˜60 nucleotides.
Trans cleavage of target RNAs demonstrated [^{xxxix}].
Binding sites and structural requirements not fully determined, although no sequences 5′of
cleavage site are required. Folded ribozyme contains a pseudoknot structure [^{xl}].
Reaction mechanism: attack by 2′-OH 5′ to the scissile bond to generate cleavage products
with 2′,3′-cyclic phosphate and 5′-OH ends.
Only 2 known members of this class. Found in human HDV.
Circular form of HDV is active and shows increased nuclease stability [^{xli}]
[ⁱ ]Michel, Francois; Westhof, Eric. Slippery substrates. Nat. Struct. Biol. (1994), 1(1), 5-7.
[ⁱⁱ ]Lisacek, Frederique; Diaz, Yolande; Michel, Francois. Automatic identification of group I intron cores in genomic DNA sequences. J. Mol. Biol. (1994), 235(4), 1206-17.
[ⁱⁱⁱ ]Herschlag, Daniel; Cech, Thomas R.. Catalysis of RNA cleavage by the Tetrahymena thermophila ribozyme. 1. Kinetic description of the reaction of an RNA substrate complementary to the active site. Biochemistry (1990), 29(44), 10159-71.
[^{iv} ]Herschlag, Daniel; Cech, Thomas R.. Catalysis of RNA cleavage by the Tetrahymena thermophila ribozyme. 2. Kinetic description of the reaction of an RNA substrate that forms a mismatch at the active site. Biochemistry (1990), 29(44), 10172-80.
[^{v} ]Knitt, Deborah S.; Herschlag, Daniel. pH Dependencies of the Tetrahymena Ribozyme Reveal an Unconventional Origin of an Apparent pKa. Biochemistry (1996), 35(5), 1560-70.
[^{vi} ]Bevilacqua, Philip C.; Sugimoto, Naoki; Turner, Douglas H.. A mechanistic framework for the second step of splicing catalyzed by the Tetrahymena ribozyme. Biochemistry (1996), 35(2), 648-58.
[^{vii} ]Li, Yi; Bevilacqua, Philip C.; Mathews, David; Turner, Douglas H.. Thermodynamic and activation parameters for binding of a pyrene-labeled substrate by the Tetrahymena ribozyme: docking is not diffusion-controlled and is driven by a favorable entropy change. Biochemistry (1995), 34(44), 14394-9.
[^{viii} ]Banerjee, Aloke Raj; Turner, Douglas H.. The time dependence of chemical modification reveals slow steps in the folding of a group I ribozyme. Biochemistry (1995), 34(19), 6504-12.
[^{ix} ]Zarrinkar, Patrick P.; Williamson, James R.. The P9.1-P9.2 peripheral extension helps guide folding of the Tetrahymena ribozyme. Nucleic Acids Res. (1996), 24(5), 854-8.
[^{x} ]Strobel, Scott A.; Cech, Thomas R.. Minor groove recognition of the conserved G.cntdot.U pair at the Tetrahymena ribozyme reaction site. Science (Washington, D.C.) (1995), 267(5198), 675-9.
[^{xi} ]Strobel, Scott A.; Cech, Thomas R.. Exocyclic Amine of the Conserved G.cntdot.U Pair at the Cleavage Site of the Tetrahymena Ribozyme Contributes to 5′-Splice Site Selection and Transition State Stabilization. Biochemistry (1996), 35(4), 1201-11.
[^{xii} ]Sullenger, Bruce A.; Cech, Thomas R.. Ribozyme-mediated repair of defective mRNA by targeted trans-splicing. Nature (London) (1994), 371(6498), 619-22.
[^{xiii} ]Robertson, H. D.; Altman, S.; Smith, J. D. J. Biol. Chem., 247, 5243-5251 (1972).
[^{xiv} ]Forster, Anthony C.; Altman, Sidney. External guide sequences for an RNA enzyme. Science (Washington, D.C., 1883-) (1990), 249(4970), 783-6.
[^{xv} ]Yuan, Y.; Hwang, E. S.; Altman, S. Targeted cleavage of mRNA by human RNase P. Proc. Natl. Acad. Sci. USA (1992) 89, 8006-10.
[^{xvi} ]Harris, Michael E.; Pace, Norman R.. Identification of phosphates involved in catalysis by the ribozyme RNase P RNA. RNA (1995), 1(2), 210-18.
[^{xvii} ]Pan, Tao; Loria, Andrew; Zhong, Kun. Probing of tertiary interactions in RNA: 2′-hydroxyl-base contacts between the RNase P RNA and pre-tRNA. Proc. Natl. Acad. Sci. U.S.A. (1995), 92(26), 12510-14.
[^{xviii} ]Pyle, Anna Marie; Green, Justin B.. Building a Kinetic Framework for Group II Intron Ribozyme Activity: Quantitation of Interdomain Binding and Reaction Rate. Biochemistry (1994), 33(9), 2716-25.
[^{xix} ]Michels, William J. Jr.; Pyle, Anna Marie. Conversion of a Group II Intron into a New Multiple-Turnover Ribozyme that Selectively Cleaves Oligonucleotides: Elucidation of Reaction Mechanism and Structure/Function Relationships. Biochemistry (1995), 34(9), 2965-77.
[^{xx} ]Zimmerly, Steven; Guo, Huatao; Eskes, Robert; Yang, Jian; Perlman, Philip S.; Lambowitz, Alan M.. A group II intron RNA is a catalytic component of a DNA endonuclease involved in intron mobility. Cell (Cambridge, Mass.) (1995), 83(4), 529-38.
[^{xxi} ]Griffin, Edmund A., Jr.; Qin, Zhifeng; Michels, Williams J., Jr.; Pyle, Anna Marie. Group II intron ribozymes that cleave DNA and RNA linkages with similar efficiency, and lack contacts with substrate 2′-hydroxyl groups. Chem. Biol. (1995), 2(11), 761-70.
[^{xxii} ]Michel, Francois; Ferat, Jean Luc. Structure and activities of group II introns. Annu. Rev. Biochem. (1995), 64, 435-61.
[^{xxiii} ]Abramovitz, Dana L.; Friedman, Richard A.; Pyle, Anna Marie. Catalytic role of 2′-hydroxyl groups within a group II intron active site. Science (Washington, D.C.) (1996), 271(5254), 1410-13.
[^{xxiv} ]Daniels, Danette L.; Michels, William J., Jr.; Pyle, Anna Marie. Two competing pathways for self-splicing by group II introns: a quantitative analysis of in vitro reaction rates and products. J. Mol. Biol. (1996), 256(1), 31-49.
[^{xxv} ]Guo, Hans C. T.; Collins, Richard A.. Efficient trans-cleavage of a stem-loop RNA substrate by a ribozyme derived from Neurospora VS RNA. EMBO J. (1995), 14(2), 368-76.
[^{xxvi} ]Scott, W. G., Finch, J. T., Aaron, K. The crystal structure of an all RNA hammerhead ribozyme:Aproposed mechanism for RNA catalytic cleavage. Cell, (1995), 81, 991-1002.
[^{xvii} ]McKay, Structure and function of the hammerhead ribozyme: an unfinished story. RNA, (1996), 2, 395-403.
[^{xviii} ]Long, D., Uhlenbeck, 0., Hertel, K. Ligation with hammerhead ribozymes. U.S. Pat. No. 5,633,133.
[^{xvix} ]Hertel, K. J., Herschlag, D., Uhlenbeck, O. A kinetic and thermodynamic framework for the hammerhead ribozyme reaction. Biochemistry, (1994) 33, 3374-3385.Beigelman, L., et al., Chemical modifications of hammerhead ribozymes. J. Biol. Chem., (1995) 270, 25702-25708.
[^{xxx} ]Beigelman, L., et al., Chemical modifications of hammerhead ribozymes. J. Biol. Chem., (1995) 270, 25702-25708.
[^{xxxi} ]Hampel, Arnold; Tritz, Richard; Hicks, Margaret; Cruz, Phillip. ‘Hairpin’ catalytic RNA model: evidence for helixes and sequence requirement for substrate RNA. Nucleic Acids Res. (1990), 18(2), 299-304.
[^{xxxii} ]Chowrira, Bharat M.; Berzal-Herranz, Alfredo; Burke, John M.. Novel guanosine requirement for catalysis by the hairpin ribozyme. Nature (London) (1991), 354(6351), 320-2.
[^{xxxiii} ]Berzal-Herranz, Alfredo; Joseph, Simpson; Chowrira, Bharat M.; Butcher, Samuel E.; Burke, John M.. Essential nucleotide sequences and secondary structure elements of the hairpin ribozyme. EMBO J. (1993), 12(6), 2567-73.
[^{xxxiv} ]Joseph, Simpson; Berzal-Herranz, Alfredo; Chowrira, Bharat M.; Butcher, Samuel E.. Substrate selection rules for the hairpin ribozyme determined by in vitro selection, mutation, and analysis of mismatched substrates. Genes Dev. (1993), 7(1), 130-8.
[^{xxxv} ]Berzal-Herranz, Alfredo; Joseph, Simpson; Burke, John M.. In vitro selection of active hairpin ribozymes by sequential RNA-catalyzed cleavage and ligation reactions. Genes Dev. (1992), 6(1), 129-34.
[^{xxxvi} ]Hegg, Lisa A.; Fedor, Martha J.. Kinetics and Thermodynamics of Intermolecular Catalysis by Hairpin Ribozymes. Biochemistry (1995), 34(48), 15813-28.
[^{xxxvii} ]Grasby, Jane A.; Mersmann, Karin; Singh, Mohinder; Gait, Michael J.. Purine Functional Groups in Essential Residues of the Hairpin Ribozyme Required for Catalytic Cleavage of RNA. Biochemistry (1995), 34(12), 4068-76.
[^{xxxviii} ]Schmidt, Sabine; Beigelman, Leonid; Karpeisky, Alexander; Usman, Nassim; Sorensen, Ulrik S.; Gait, Michael J.. Base and sugar requirements for RNA cleavage of essential nucleoside residues in internal loop B of the hairpin ribozyme: implications for secondary structure. Nucleic Acids Res. (1996), 24(4), 573-81.
[^{xxix} ]Perrotta, Anne T.; Been, Michael D.. Cleavage of oligoribonucleotides by a ribozyme derived from the hepatitis .delta. virus RNA sequence. Biochemistry (1992), 31(1), 16-21.
[^{xl} ]Perrotta, Anne T.; Been, Michael D.. A pseudoknot-like structure required for efficient self-cleavage of hepatitis delta virus RNA. Nature (London) (1991), 350(6317), 434-6.
[^{xli} ]Puttaraju, M.; Perrotta, Anne T.; Been, Michael D.. A circular trans-acting hepatitis delta virus ribozyme. Nucleic Acids Res. (1993), 21(18), 4253-8.

TABLE II
2.5 μmol RNA Synthesis Cycle

| | | | Wait |
|---|---|---|---|
| Reagent | Equivalents | Amount | Time* |
| Phosphoramidites | 6.5 | 163 μL | 2.5 |
| S-Ethyl Tetrazole | 23.8 | 238 μL | 2.5 |
| Acetic Anhydride | 100 | 233 μL | 5 sec |
| N-Methyl Imidazole | 186 | 233 μL | 5 sec |
| TCA | 83.2 | 1.73 mL | 21 sec |
| Iodine | 8.0 | 1.18 mL | 45 sec |
| Acetonitrile | NA | 6.67 mL | NA |

*Wait time does not include contact time during delivery.

TABLE III
Ribozyme Selection Characteristics

| Characteristic | Number | |
|---|---|---|
| HCV Genome Length | 9436 | kb |
| All Hammerhead Cleavage Sites* | 1300 | |
| Conserved Region Hammerhead Cleavage | 23 | |
| Sites** | | |

*HCV Genotype 1b was the prototype strain
**Based on sequence alignments from HCV genotype 1a, 1b, 1c, 2a, 2b, 2c 3a, 3b, 4a, 4c, 4f, 5a, and 6a

TABLE IV
Hammerhead Ribozymes Derived from Conserved Regions of
the HCV Genome

| | | |
|---|---|---|
| Name | Substrate | Ribozyme Sequence |

| | | |
|---|---|---|
| 5′ NCR | | |
| HCV.5-50 | CUACUGU C UUCACGC | GCGUGAA CUGAUGAGGCCGUUAGGCCGAA ACAGUAG |
| HCV.5-67 | AAAGCGU C UAGCCAU | AUGGCUA CUGAUGAGGCCGUUAGGCCGAA ACGCUUU |
| HCV.5-69 | AGCGUCU A GCCAUGG | CCAUGGC CUGAUGAGGCCGUUAGGCCGAA AGACGCU |
| HCV.5-92 | UGAGUGU C GUGCAGC | GCUGCAC CUGAUGAGGCCGUUAGGCCGAA ACACUCA |
| HCV.5-130 | GAGCCAU A GUGGUCU | AGACCAC CUGAUGAGGCCGUUAGGCCGAA AUGGCUC |
| HCV.5-136 | UAGUGGU C UGCGGAA | UUCCGCA CUGAUGAGGCCGUUAGGCCGAA ACCACUA |
| HCV.5-153 | GGUGAGU A CACCGGA | UCCGGUG CUGAUGAGGCCGUUAGGCCGAA ACUCACC |
| HCV 5-180 | ACCGGGU C CUUUCUU | AAGAAAG CUGAUGAGGCCGUUAGGCCGAA ACCCGGU |
| HCV.5-183 | GGGUCCU U UCUUGGA | UCCAAGA CUGAUGAGGCCGUUAGGCCGAA AGGACCC |
| HCV.5-184 | GGUCCUU U CUUGGAU | AUCCAAG CUGAUGAGGCCGUUAGGCCGAA AAGGACC |
| HCV.5-258 | GUUGGGU C GCGAAAG | CUUUCGC CUGAUGAGGCCGUUAGGCCGAA ACCCAAC |
| HCV 5-270 | AAGGGCU U GUGGUAC | GUACCAC CUGAUGAGGCCGUUAGGCCGAA AGGCCUU |
| HCV.5-294 | GGGUGCU U GCGAGUG | CACUCGC CUGAUGAGGCCGUUAGGCCGAA AGCACCC |
| HCV.5-313 | GGGAGGU C UCGUAGA | UCUACGA CUGAUGAGGCCGUUAGGCCGAA ACCUCCC |
| HCV.5-315 | GAGGUCU C GUAGACC | GGUCUAC CUGAUGAGGCCGUUAGGCCGAA AGACCUC |
| HCV.5-318 | GUCUCGU A GACCGUG | CACGGUC CUGAUGAGGCCGUUAGGCCGAA ACGAGAC |
| Core Region | | |
| HCV.C-30 | UAAACCU C AAAGAAA | UUUCUUU CUGAUGAGGCCGUUAGGCCGAA AGGUUUA |
| HCV.C-48 | CAAACGU A ACACCAA | UUGGUGU CUGAUGAGGCCGUUAGGCCGAA ACGUUUG |
| HCV C-60 | CAACCGU C GCCCACA | UGUGGGC CUGAUGAGGCCGUUAGGCCGAA ACGGUUG |
| HCV.C-175 | GAGCGGU C ACAACCU | AGGUUGU CUGAUGAGGCCGUUAGGCCGAA ACCGCUC |
| HCV C-374 | GUAAGGU C AUCGAUA | UAUCGAU CUGAUGAGGCCGUUAGGCCGAA ACCUUAC |
| 3′ NCR | | |
| HCV.3-118 | UUUUUUU U UUUUUUU | AAAAAAA CUGAUGAGGCCGUUAGGCCGAA AAAAAAA |
| HCV.3-145 | GGUGGCU C CAUCUUA | UAAGAUG CUGAUGAGGCCGUUAGGCCGAA AGCCACC |
| HCV.3-149 | GCUCCAU C UUAGCCC | GGGCUAA CUGAUGAGGCCGUUAGGCCGAA AUGGAGC |
| HCV.3-151 | UCCAUCU U AGCCCUA | UAGGGCU CUGAUGAGGCCGUUAGGCCGAA AGAUGGA |
| HCV.3-152 | CCAUCUU A GCCCUAG | CUAGGGC CUGAUGAGGCCGUUAGGCCGAA AAGAUGG |
| HCV.3-158 | UAGCCCU A GUCACGG | CCGUGAG CUGAUGAGGCCGUUAGGCCGAA AGGGCUA |
| HCV 3-161 | CCCUAGU C ACGGCUA | UAGCCGU CUGAUGAGGCCGUUAGGCCGAA ACUAGGG |
| HCV.3-168 | CACGGCU A GCUGUGA | UCACAGC CUGAUGAGGCCGUUAGGCCGAA AGCCGUG |
| HCV.3-181 | GAAAGGU C CGUGAGC | GCUCACG CUGAUGAGGCCGUUAGGCCGAA ACCUUUC |

TABLE V
HCV Hammerhead Ribozyme and Target Sequence

| | | | | |
|---|---|---|---|---|
| | | Nt. | | |
| No. | Name | Pos. | Hammerhead Ribozyme | Substrate |

| | | | | |
|---|---|---|---|---|
| 1 | HCV-27 | 27 | UAUGGUG CUGAUGAG X CGAA AGUGUCG | CGACACU C CACCAUA |
| 2 | HCV-114 | 114 | GGUCCUG CUGAUGAG X CGAA AGGCUGC | GCAGCCU C CAGGACC |
| 3 | HCV-128 | 128 | CUCCCGG CUGAUGAG X CGAA AGGGGGG | CCCCCCU C CCGGGAG |
| 4 | HCV-148 | 148 | UUCCGCA CUGAUGAG X CGAA ACCACUA | UAGUGGU C UGCGGAA |
| 5 | HCV-165 | 165 | UCCGGUG CUGAUGAG X CGAA ACUCACC | GGUGAGU A CACCGGA |
| 6 | HCV-175 | 175 | UCCUGGC CUGAUGAG X CGAA AUUCCGG | CCGGAAU U GCCAGGA |
| 7 | HCV-199 | 199 | UUGAUCC CUGAUGAG X CGAA AGAAAGG | CCUUUCU U GGAUCAA |
| 8 | HCV-213 | 213 | AGGCAUU CUGAUGAG X CGAA AGCGGGU | ACCCGCU C AAUGCCU |
| 9 | HCV-252 | 252 | ACUCGGC CUGAUGAG X CGAA AGCAGUC | GACUGCU A GCCGAGU |
| 10 | HCV-260 | 260 | CCAACAC CUGAUGAG X CGAA ACUCGGC | GCCGAGU A GUGUUGG |
| 11 | HCV-265 | 265 | GCGACCC CUGAUGAG X CGAA ACACUAC | GUAGUGU U GGGUCGC |
| 12 | HCV-270 | 270 | CUUUCGC CUGAUGAG X CGAA ACCCAAC | GUUGGGU C GCGAAAG |
| 13 | HCV-288 | 288 | CAGGCAG CUGAUGAG X CGAA ACCACAA | UUGUGGU A CUGCCUG |
| 14 | HCV-298 | 298 | AGCACCC CUGAUGAG X CGAA AUCAGGC | GCCUGAU A GGGUGCU |
| 15 | HCV-306 | 306 | CACUCGC CUGAUGAG X CGAA AGCACCC | GGGUGCU U GCGAGUG |
| 16 | HCV-325 | 325 | UCUACGA CUGAUGAG X CGAA ACCUCCC | GGGAGGU C UCGUAGA |
| 17 | HCV-327 | 327 | GGUCUAC CUGAUGAG X CGAA AGACCUC | GAGGUCU C GUAGACC |
| 18 | HCV-330 | 330 | CACGGUC CUGAUGAG X CGAA ACGAGAC | GUCUCGU A GACCGUG |
| 19 | HCV-407 | 407 | GGAACUU CUGAUGAG X CGAA ACGUCCU | AGGACGU C AAGUUCC |
| 20 | HCV-412 | 412 | GCCCGGG CUGAUGAG X CGAA ACUUGAC | GUCAAGU U CCCGGGC |
| 21 | HCV-413 | 413 | CGCCCGG CUGAUGAG X CGAA AACUUGA | UCAAGUU C CCGGGCG |
| 22 | HCV-426 | 426 | ACGAUCU CUGAUGAG X CGAA ACCACCG | CGGUGGU C AGAUCGU |
| 23 | HCV-472 | 472 | CACACCC CUGAUGAG X CGAA ACGUGGG | CCCACGU U GGGUGUG |
| 24 | HCV-489 | 489 | GUCUUCC CUGAUGAG X CGAA AGUCGCG | CGCGACU A GGAAGAC |
| 25 | HCV-498 | 498 | CGUUCGG CUGAUGAG X CGAA AGUCUUC | GAAGACU U CCGAACG |
| 26 | HCV-499 | 499 | CCGUUCG CUGAUGAG X CGAA AAGUCUU | AAGACUU C CGAACGG |
| 27 | HCV-508 | 508 | AGGUUGC CUGAUGAG X CGAA ACCGUUC | GAACGGU C GCAACCU |
| 28 | HCV-534 | 534 | UUGGGGA CUGAUGAG X CGAA AGGUUGU | ACAACCU A UCCCCAA |
| 29 | HCV-536 | 536 | CCUUGGG CUGAUGAG X CGAA AUAGGUU | AACCUAU C CCCAAGG |
| 30 | HCV-546 | 546 | GGUCGGC CUGAUGAG X CGAA AGCCUUG | CAAGGCU C GCCGACC |
| 31 | HCV-561 | 561 | CAGGCCC CUGAUGAG X CGAA ACCCUCG | CGAGGGU A GGGCCUG |
| 32 | HCV-573 | 573 | CCAGGCU CUGAUGAG X CGAA AGCCCAG | CUGGGCU C AGCCUGG |
| 33 | HCV-583 | 583 | CCAAGGG CUGAUGAG X CGAA ACCCAGG | CCUGGGU A CCCUUGG |
| 34 | HCV-588 | 588 | AGGGGCC CUGAUGAG X CGAA AGGGUAC | GUACCCU U GGCCCCU |
| 35 | HCV-596 | 596 | UGCCAUA CUGAUGAG X CGAA AGGGGCC | GGCCCCU C UAUGGCA |
| 36 | HCV-598 | 598 | AUUGCCA CUGAUGAG X CGAA AGAGGGG | CCCCUCU A UGGCAAU |
| 37 | HCV-632 | 632 | GUGACAG CUGAUGAG X CGAA AGCCAUC | GAUGGCU C CUGUCAC |
| 38 | HCV-637 | 637 | GCGGGGU CUGAUGAG X CGAA ACAGGAG | CUCCUGU C ACCCCGC |
| 39 | HCV-649 | 649 | AGGCCGG CUGAUGAG X CGAA AGCCGCG | CGCGGCU C CCGGCCU |
| 40 | HCV-657 | 657 | CCCCAAC CUGAUGAG X CGAA AGGCCGG | CCGGCCU A GUUGGGG |
| 41 | HCV-660 | 660 | GGGCCCC CUGAUGAG X CGAA ACUAGGC | GCCUAGU U GGGGCCC |
| 42 | HCV-696 | 696 | UUACCCA CUGAUGAG X CGAA AUUGCGC | GCGCAAU C UGGGUAA |
| 43 | HCv-707 | 707 | UAUCGAU CUGAUGAG X CGAA ACCUUAC | GUAAGGU C AUCGAUA |
| 44 | HCV-710 | 710 | GGGUAUC CUGAUGAG X CGAA AUGACCU | AGGUCAU C GAUACCC |
| 45 | HCV-714 | 714 | GUGAGGG CUGAUGAG X CGAA AUCGAUG | CAUCGAU A CCCUCAC |
| 46 | HCV-730 | 730 | GUCGGCG CUGAUGAG X CGAA AGCCGCA | UGCGGCU U CGCCGAC |
| 47 | HCV-731 | 731 | GGUCGGC CUGAUGAG X CGAA AAGCCGC | GCGGCUU C GCCGACC |
| 48 | HCV-748 | 748 | CGGAAUG CUGAUGAG X CGAA ACCCCAU | AUGGGGU A CAUUCCG |
| 49 | HCV-752 | 752 | CGAGCGG CUGAUGAG X CGAA AUGUACC | GGUACAU U CCGCUCG |
| 50 | HCV-753 | 753 | ACGAGCG CUGAUGAG X CGAA AAUGUAC | GUACAUU C CGCUCGU |
| 51 | HCV-758 | 758 | CGCCGAC CUGAUGAG X CGAA AGCGGAA | UUCCGCU C GUCGGCG |
| 52 | HCV-761 | 761 | GGGCGCC CUGAUGAG X CGAA ACGAGCG | CGCUCGU C GGCGCCC |
| 53 | HCV-773 | 773 | CGCCCCC CUGAUGAG X CGAA AGGGGGG | CCCCCCU A GGGGGCG |
| 54 | HCV-806 | 806 | GAACCCG CUGAUGAG X CGAA ACACCAU | AUGGUGU C CGGGUUC |
| 55 | HCV-812 | 812 | CCUCCAG CUGAUGAG X CGAA ACCCGGA | UCCGGGU U CUGGAGG |
| 56 | HCV-813 | 813 | UCCUCCA CUGAUGAG X CGAA AACCCGG | CCGGGUU C UGGAGGA |
| 57 | HCV-832 | 832 | UGUUGCG CUGAUGAG X CGAA AGUUCAC | GUGAACU A CGCAACA |
| 58 | HCV-847 | 847 | ACCGGGC CUGAUGAG X CGAA AGUUCCC | GGGAACU U GCCCGGU |
| 59 | HCV-855 | 855 | AAAGAGC CUGAUGAG X CGAA ACCGGGC | GCCCGGU U GCUCUUU |
| 60 | HCV-859 | 859 | AGAGAAA CUGAUGAG X CGAA AGCAACC | GGUUGCU C UUUCUCU |
| 61 | HCV-982 | 982 | UGCCUCA CUGAUGAG X CGAA ACACAAU | AUUGUGU A UGAGGCA |
| 62 | HCV-1001 | 1001 | UAUGCAU CUGAUGAG X CGAA AUCAUGC | GCAUGAU C AUGCAUA |
| 63 | HCV-1022 | 1022 | CGCAGGG CUGAUGAG X CGAA ACGCACC | GGUGCGU A CCCUGCG |
| 64 | HCV-1031 | 1031 | UCUCCCG CUGAUGAG X CGAA ACGCAGG | CCUGCGU U CGGGAGA |
| 65 | HCV-1032 | 1032 | UUCUCCC CUGAUGAG X CGAA AACGCAG | CUGCGUU C GGGAGAA |
| 66 | HCV-1048 | 1048 | ACAACGG CUGAUGAG X CGAA AGGCGUU | AACGCCU C CCGUUGU |
| 67 | HCV-1053 | 1053 | ACCCAAC CUGAUGAG X CGAA ACGGGAG | CUCCCGU U GUUGGGU |
| 68 | HCV-1056 | 1056 | GCUACCC CUGAUGAG X CGAA ACAACGG | CCGUUGU U GGGUAGC |
| 69 | HCV-1061 | 1061 | UGAGCGC CUGAUGAG X CGAA ACCCAAC | GUUGGGU A GCGCUCA |
| 70 | HCV-1127 | 1127 | GCAAGUC CUGAUGAG X CGAA ACGUGGC | GCCACGU C GACUUGC |
| 71 | HCV-1132 | 1132 | AACGAGC CUGAUGAG X CGAA AGUCGAC | GUCGACU U GCUCGUU |
| 72 | HCV-1136 | 1136 | CCCCAAC CUGAUGAG X CGAA AGCAAGU | ACUUGCU C GUUGGGG |
| 73 | HCV-1139 | 1139 | CCGCCCC CUGAUGAG X CGAA ACGAGCA | UGCUCGU U GGGGCGG |
| 74 | HCV-1153 | 1153 | GGAACAG CUGAUGAG X CGAA AAGCGGC | GCCGCUU U CUGUUCC |
| 75 | HCV-1154 | 1154 | CGGAACA CUGAUGAG X CGAA AAAGCGG | CCGCUUU C UGUUCCG |
| 76 | HCV-1158 | 1158 | AUGGCGG CUGAUGAG X CGAA ACAGAAA | UUUCUGU U CCGCCAU |
| 77 | HCV-1159 | 1159 | CAUGGCG CUGAUGAG X CGAA AACAGAA | UUCUGUU C CGCCAUG |
| 78 | HCV-1168 | 1168 | CCCCACG CUGAUGAG X CGAA ACAUGGC | GCCAUGU A CGUGGGG |
| 79 | HCV-1189 | 1189 | GAAAACG CUGAUGAG X CGAA AUCCGCA | UGCGGAU C CGUUUUC |
| 80 | HCV-1193 | 1193 | CGAGGAA CUGAUGAG X CGAA ACGGAUC | GAUCCGU U UUCCUCG |
| 81 | HCV-1194 | 1194 | ACGAGGA CUGAUGAG X CGAA AACGGAU | AUCCGUU U UCCUCGU |
| 82 | HCV-1195 | 1195 | GACGAGG CUGAUGAG X CGAA AAACGGA | UCCGUUU U CCUCGUC |
| 83 | HCV-1196 | 1196 | AGACGAG CUGAUGAG X CGAA AAAACGG | CCGUUUU C CUCGUCU |
| 84 | HCV-1200 | 1280 | GACCUGA CUGAUGAG X CGAA ACAUGGG | GCCAUGU A UCAGGUC |
| 85 | HCV-1282 | 1282 | GUGACCU CUGAUGAG X CGAA AUACAUG | CAUGUAU C AGGUCAC |
| 86 | HCV-1287 | 1207 | AUGCGGU CUGAUGAG X CGAA ACCUGAU | AUCAGGU C ACCGCAU |
| 87 | HCV-1373 | 1373 | UAUCCAC CUGAUGAG X CGAA ACAGCUU | AAGCUGU C GUGGAUA |
| 88 | HCV-1380 | 1380 | GCCACCA CUGAUGAG X CGAA AUCCACG | CGUGGAU A UGGUGGC |
| 89 | HCV-1406 | 1406 | CCGCUAG CUGAUGAG X CGAA ACUCCCC | GGGGAGU C CUAGCGG |
| 90 | HCV-1409 | 1409 | GGCCCGC CUGAUGAG X CGAA AGGACUC | GAGUCCU A GCGGGCC |
| 91 | HCV-1418 | 1418 | AGUAGGC CUGAUGAG X CGAA AGGCCCG | CGGGCCU U GCCUACU |
| 92 | HCV-1423 | 1423 | GGAAUAG CUGAUGAG X CGAA AGGCAAG | CUUGCCU A CUAUUCC |
| 93 | HCV-1426 | 1426 | CAUGGAA CUGAUGAG X CGAA AGUAGGC | GCCUACU A UUCCAUG |
| 94 | HCV-1428 | 1428 | ACCAUGG CUGAUGAG X CGAA AUAGUAG | CUACUAU U CCAUGGU |
| 95 | HCV-1429 | 1429 | CACCAUG CUGAUGAG X CGAA AAUAGUA | UACUAUU C CAUGGUG |
| 96 | HCV-1727 | 1727 | ACUUGUC CUGAUGAG X CGAA AUGGAGC | GCUCCAU C GACAAGU |
| 97 | HCV-1735 | 1735 | CUGAGCG CUGAUGAG X CGAA ACUUGUC | GACAAGU U CGCUCAG |
| 98 | HCV-1736 | 1736 | CCUGAGC CUGAUGAG X CGAA AACUUGU | ACAAGUU C GCUCAGG |
| 99 | HCV-1740 | 1740 | CAUCCCU CUGAUGAG X CGAA AGCGAAC | GUUCGCU C AGGGAUG |
| 100 | HCV-1757 | 1757 | UAUAGGU CUGAUGAG X CGAA AUGGGGC | GCCCCAU C ACCUAUA |
| 101 | HCV-1762 | 1762 | CUCGGUA CUGAUGAG X CGAA AGGUGAU | AUCACCU A UACCGAG |
| 102 | HCV-1795 | 1795 | CCAGCAG CUGAUGAG X CGAA AAGGCCU | AGGCCUU A CUGCUGG |
| 103 | HCV-1806 | 1806 | GGUGCGU CUGAUGAG X CGAA AUGCCAG | CUGGCAU U ACGCACC |
| 104 | HCV-1807 | 1807 | AGGUGCG CUGAUGAG X CGAA AAUGCCA | UGGCAUU A CGCACCU |
| 105 | HCV-1815 | 1815 | CACUGCC CUGAUGAG X CGAA AGGUGCG | CGCACCU C GGCAGUG |
| 106 | HCV-1827 | 1827 | GGUACGA CUGAUGAG X CGAA ACCACAC | GUGUGGU A UCGUACC |
| 107 | HCV-1829 | 1829 | CAGGUAC CUGAUGAG X CGAA AUACCAC | GUGGUAU C GUACCUG |
| 108 | HCV-1832 | 1832 | ACGCAGG CUGAUGAG X CGAA ACGAUAC | GUAUCGU A CCUGCGU |
| 109 | HCV-1840 | 1840 | CACCUGC CUGAUGAG X CGAA ACGCAGG | CCUGCGU C GCAGGUG |
| 110 | HCV-1854 | 1854 | UACACUG CUGAUGAG X CGAA ACCACAC | GUGUGGU C CAGUGUA |
| 111 | HCV-1883 | 1883 | CCACUAC CUGAUGAG X CGAA ACAGGGC | GCCCUGU U GUAGUGG |
| 112 | HCV-1886 | 1886 | UCCCCAC CUGAUGAG X CGAA ACAACAG | CUGUUGU A GUGGGGA |
| 113 | HCV-1902 | 1902 | CCGGACC CUGAUGAG X CGAA AUCGGUC | GACCGAU C GGUCCGG |
| 114 | HCV-1906 | 1906 | GGCACCG CUGAUGAG X CGAA ACCGAUC | GAUCGGU C CGGUGCC |
| 115 | HCV-1917 | 1917 | UUAUACG CUGAUGAG X CGAA AGGGGCA | UGCCCCU A CGUAUAA |
| 116 | HCV-1921 | 1921 | CCAGUUA CUGAUGAG X CGAA ACGUAGG | CCUACGU A UAACUGG |
| 117 | HCV-1923 | 1923 | CCCCAGU CUGAUGAG X CGAA AUACGUA | UACGUAU A ACUGGGG |
| 118 | HCV-1990 | 1990 | ACAGCCA CUGAUGAG X CGAA ACCAGUU | AACUGGU U UGGCUGU |
| 119 | HCV-1991 | 1991 | UACAGCC CUGAUGAG X CGAA AACCAGU | ACUGGUU U GGCUGUA |
| 120 | HCV-1998 | 1998 | AUCCAUG CUGAUGAG X CGAA ACAGCCA | UGGCUGU A CAUGGAU |
| 121 | HCV-2043 | 2043 | UUGCACG CUGAUGAG X CGAA AGGGCCC | GGGCCCU C CGUGCAA |
| 122 | HCV-2054 | 2054 | CCCCCCC CUGAUGAG X CGAA AUGUUGC | GCAACAU C GGGGGGG |
| 123 | HCV-2063 | 2063 | GGUUGCC CUGAUGAG X CGAA ACCCCCC | GGGGGGU C GGCAACC |
| 124 | HCV-2072 | 2072 | UCAAGGU CUGAUGAG X CGAA AGGUUGC | GCAACCU C ACCUUGA |
| 125 | HCV-2077 | 2077 | GCAGGUC CUGAUGAG X CGAA AGGUGAG | CUCACCU U GACCUGC |
| 126 | HCV-2121 | 2121 | UUUGUGU CUGAUGAG X CGAA AGUGGCC | GGCCACU U ACACAAA |
| 127 | HCV-2122 | 2122 | UUUUGUG CUGAUGAG X CGAA AAGUGGC | GCCACUU A CACAAAA |
| 128 | HCV-2137 | 2137 | UGGCCCC CUGAUGAG X CGAA AGCCACA | UGUGGCU C GGGGCCA |
| 129 | HCV-2149 | 2149 | AGGUGUU CUGAUGAG X CGAA ACCAUGG | CCAUGGU U AACACCU |
| 130 | HCV-2150 | 2150 | UAGGUGU CUGAUGAG X CGAA AACCAUG | CAUGGUU A ACACCUA |
| 131 | HCV-2219 | 2219 | CCUUAAA CUGAUGAG X CGAA AUGGUAA | UUACCAU C UUUAAGG |
| 132 | HCV-2221 | 2221 | AACCUUA CUGAUGAG X CGAA AGAUGGU | ACCAUCU U UAAGGUU |
| 133 | HCV-2261 | 2261 | CAGCACU CUGAUGAG X CGAA AGCCUGU | ACAGGCU U AGUGCUG |
| 134 | HCV-2262 | 2262 | GCAGCAC CUGAUGAG X CGAA AAGCCUG | CAGGCUU A GUGCUGC |
| 135 | Hcv-2295 | 2295 | AGGUCGC CUGAUGAG X CGAA ACGCUCU | AGAGCGU U GCGACCU |
| 136 | HCV-2320 | 2320 | GAGCUCC CUGAUGAG X CGAA AUCUGUC | GACAGAU C GGAGCUC |
| 137 | HCV-2327 | 2327 | GCGGGCU CUGAUGAG X CGAA AGCUCCG | CGGAGCU C AGCCCGC |
| 138 | HCV-2344 | 2344 | UGUCGUG CUGAUGAG X CGAA ACAGGAG | CUGCUGU C CACGACA |
| 139 | HCV-2417 | 2417 | UCUGAUG CUGAUGAG X CGAA AGGUGGA | UCCACCU C CAUCAGA |
| 140 | HCV-2421 | 2421 | AUGUUCU CUGAUGAG X CGAA AUGGAGG | CCUCCAU C AGAACAU |
| 141 | HCV-2429 | 2429 | CGUCCAC CUGAUGAG X CGAA AUGUUCU | AGAACAU C GUGGACG |
| 142 | HCV-2534 | 2534 | AGGCACA CUGAUGAG X CGAA ACGCGCG | CGCGCGU C UGUGCCU |
| 143 | HCV-2585 | 2585 | GGUUCUC CUGAUGAG X CGAA AGGGCGG | CCGCCCU A GAGAACC |
| 144 | HCV-2600 | 2600 | CGUUGAG CUGAUGAG X CGAA ACCACCA | UGGUGGU C CUCAACG |
| 145 | HCV-2603 | 2603 | CCGCGUU CUGAUGAG X CGAA AGGACCA | UGGUCCU C AACGCGG |
| 146 | HCV-2673 | 2671 | CUUGAUG CUGAUGAG X CGAA ACCAGGC | GCCUGGU A CAUCAAG |
| 147 | ECV-2675 | 2675 | UGCCCUU CUGAUGAG X CGAA AUGUACC | GGUACAU C AAGGGCA |
| 148 | HCV-2690 | 2690 | CCCCAGG CUGAUGAG X CGAA ACCAGCC | GGCUGGU C CCUGGGG |
| 149 | HCV-2704 | 2704 | CAGAGCA CUGAUGAG X CGAA AUGCCGC | GCGGCAU A UGGUCUG |
| 150 | HCV-2709 | 2709 | CCGUACA CUGAUGAG X CGAA AGCAUAU | AUAUGCU C UGUACGG |
| 151 | HCV-2713 | 2713 | CACGCCG CUGAUGAG X CGAA ACAGAGC | GCUCUGU A CGGCGUG |
| 152 | HCV-2738 | 2738 | CCAGCAG CUGAUGAG X CGAA AGCAGGA | UCCUGCU C CUGCUGG |
| 153 | HCV-2763 | 2763 | AUGGCGU CUGAUGAG X CGAA AGCCCGU | ACGGGCU U ACGCCAU |
| 154 | HCV-2764 | 2764 | CAUGGCG CUGAUGAG X CGAA AAGCCCG | CGGGCUU A CGCCAUG |
| 155 | HCV-2878 | 2878 | GUAUUGU CUGAUGAG X CGAA ACCACCA | UGGUGGU U ACAAUAC |
| 156 | HCV-2879 | 2879 | AGUAUUG CUGAUGAG X CGAA AACCACC | GGUGGUU A CAAUACU |
| 157 | HCV-2884 | 2884 | GAUAAAG CUGAUGAG X CGAA AUUGUAA | UUACAAU A CUUUAUC |
| 158 | HCV-2887 | 2887 | GGUGAUA CUGAUGAG X CGAA AGUAUUG | CAAUACU U UAUCACC |
| 159 | HCV-2888 | 2888 | UGGUGAU CUGAUGAG X CGAA AAGUAUU | AAUACUU U AUCACCA |
| 160 | HCV-2910 | 2910 | ACGCACA CUGAUGAG X CGAA AUGCGCC | GGCGCAU U UGUGCGU |
| 161 | HCV-2911 | 2911 | CACGCAC CUGAUGAG X CGAA AAUGCGC | GCGCAUU U GUGCGUG |
| 162 | HCV-2924 | 2924 | GAGGGGG CUGAUGAG X CGAA ACCCACA | UGUGGGU C CCCCCUC |
| 163 | HCV-2931 | 2933 | ACAUUGA CUGAUGAG X CGAA AGGGGGG | CCCCCCU C UCAAUGU |
| 164 | HCV-2933 | 2933 | GGACAUU CUGAUGAG X CGAA AGAGGGG | CCCCUCU C AAUGUCC |
| 165 | HCV-2939 | 2939 | CCCCCCG CUGAUGAG X CGAA ACAUUGA | UCAAUGU C CGGGGGG |
| 166 | HCV-2958 | 2958 | AGGAUGA CUGAUGAG X CGAA AGCAUCG | CGAUGCU A UCAUCCU |
| 167 | HCV-2960 | 2960 | GGAGGAU CUGAUGAG X CGAA AUAGCAU | AUGCUAU C AUCCUCC |
| 168 | HCV-2963 | 2963 | UGAGGAG CUGAUGAG X CGAA AUGAUAG | CUAUCAU C CUCCUCA |
| 169 | HCV-2966 | 2966 | AUGUGAG CUGAUGAG X CGAA AGGAUGA | UCAUCCU C CUCACAU |
| 170 | HCV-2969 | 2969 | CACAUGU CUGAUGAG X CGAA AGGAGGA | UCCUCCU C ACAUGUG |
| 171 | HCV-3059 | 3059 | UCGCAGU CUGAUGAG X CGAA AUGGCAG | CUGCCAU A ACUGCGA |
| 172 | HCV-3138 | 3138 | UGGACGU CUGAUGAG X CGAA AUGGCCU | AGGCCAU U ACGUCCA |
| 173 | HCV-3139 | 3139 | UUGGACG CUGAUGAG X CGAA AAUGGCC | GGCCAUU A CGUCCAA |
| 174 | HCV-3143 | 3143 | CCAUUUG CUGAUGAG X CGAA ACGUAAU | AUUACGU C CAAAUGG |
| 175 | HCV-3154 | 3154 | CUUCAUG CUGAUGAG X CGAA AGGCCAU | AUGGCCU U CAUGAAG |
| 176 | HCV-3155 | 3155 | GCUUCAU CUGAUGAG X CGAA AAGGCCA | UGGCCUU C AUGAAGC |
| 177 | HCV-3209 | 3209 | AAUCCUG CUGAUGAG X CGAA AGCGGGG | CCCCGCU A CAGGAUU |
| 178 | HCV-3216 | 3216 | UGGGCCC CUGAUGAG X CGAA AUCCUGU | ACAGGAU U GGGCCCA |
| 179 | HCV-3233 | 3233 | GGUCUCG CUGAUGAG X CGAA AGGCCCG | CGGGCCU A CGAGACC |
| 180 | HCV-3242 | 3242 | CCACCGC CUGAUGAG X CGAA AGGUCUC | GAGACCU U GCGGUGG |
| 181 | HCV-3263 | 3263 | AGAAGAC CUGAUGAG X CGAA ACGGGCU | AGCCCGU C GUCUUCU |
| 182 | HCV-3266 | 3266 | CAGAGAA CUGAUGAG X CGAA ACGACGG | CCGUCGU C UUCUCUG |
| 183 | HCV-3268 | 3268 | GUCAGAG CUGAUGAG X CGAA AGACGAC | GUCGUCU U CUCUGAC |
| 184 | HCV-3290 | 3290 | AGGUGAU CUGAUGAG X CGAA AUCUUGG | CCAAGAU C AUCACCU |
| 185 | HCV-3293 | 3293 | CCCAGGU CUGAUGAG X CGAA AUGAUCU | AGAUCAU C ACCUGGG |
| 186 | HCV-3329 | 3329 | CCAAGAU CUGAUGAG X CGAA AUGUCCC | GGGACAU C AUCUUGG |
| 187 | HCV-3332 | 3332 | GUCCCAA CUGAUGAG X CGAA AUGAUGU | ACAUCAU C UUGGGAC |
| 188 | HCV-3334 | 3334 | CAGUCCC CUGAUGAG X CGAA AGAUGAU | AUCAUCU U GGGACUG |
| 189 | HCV-3347 | 3347 | GGGCGGA CUGAUGAG X CGAA ACGGGCA | UGCCCGU C UCCGCCC |
| 190 | HCV-3349 | 3349 | UCGGGCG CUGAUGAG X CGAA AGACGGG | CCCGUCU C CGCCCGA |
| 191 | HCV-3371 | 3371 | CCAGAAG CUGAUGAG X CGAA AUCUCCC | GGGAGAU A CUUCUGG |
| 192 | HCV-3416 | 3416 | GGGCAAG CUGAUGAG X CGAA AGUCGCC | GGCGACU C CUUGCCC |
| 193 | HCV-3419 | 3419 | UGGGGGC CUGAUGAG X CGAA AGGAGUC | GACUCCU U GCCCCCA |
| 194 | HCV-3428 | 3428 | AGGCCGU CUGAUGAG X CGAA AUGGGGG | CCCCCAU C ACGGCCU |
| 195 | HCV-3482 | 3482 | GGCCUGU CUGAUGAG X CGAA AGGCUAG | CUAGCCU C ACAGGCC |
| 196 | HCV-3518 | 3518 | CCACUUG CUGAUGAG X CGAA ACCUCCC | GGGAGGU U CAAGUGG |
| 197 | HCV-3519 | 3519 | ACCACUU CUGAUGAG X CGAA AACCUCC | GGAGGUU C AAGUGGU |
| 198 | HCV-3527 | 3527 | CGGUGGA CUGAUGAG X CGAA ACCACUU | AAGUGGU U UCCACCG |
| 199 | HCV-3528 | 3528 | GCGGUGG CUGAUGAG X CGAA AACCACU | AGUGGUU U CCACCGC |
| 200 | HCV-3529 | 3529 | UGCGGUG CUGAUGAG X CGAA AAACCAC | GUGGUUU C CACCGCA |
| 201 | HCV-3576 | 3576 | ACGGUCC CUGAUGAG X CGAA ACACACA | UGUGUGU U GGACCGU |
| 202 | HCV-3601 | 3601 | GGUCUUU CUGAUGAG X CGAA AGCCGGC | GCCGGCU C AAAGACC |
| 203 | HCV-3611 | 3611 | GGCCGGC CUGAUGAG X CGAA AGGGUCU | AGACCCU A GCCGGCC |
| 204 | HCV-3684 | 3684 | GCCCCGG CUGAUGAG X CGAA AGGCGCA | UGCGCCU C CCGGGGC |
| 205 | HCV-3696 | 3696 | GUAAGGG CUGAUGAG X CGAA ACGCGCC | GGCGCGU U CCCUUAC |
| 206 | HCV-3697 | 3697 | UGUAAGG CUGAUGAG X CGAA AACGCGC | GCGCGUU C CCUUACA |
| 207 | HCV-3701 | 3701 | AUGGUGU CUGAUGAG X CGAA AGGGAAC | GUUCCCU U ACACCAU |
| 208 | HCV-3702 | 3702 | CAUGGUG CUGAUGAG X CGAA AAGGGAA | UUCCCUU A CACCAUG |
| 209 | HCV-3724 | 3724 | GAGGUCC CUGAUGAG X CGAA AGCUACC | GGUAGCU C GGACCUC |
| 210 | HCV-3731 | 3731 | CCAGAUA CUGAUGAG X CGAA AGGUCCG | CGGACCU C UAUCUGG |
| 211 | HCV-3733 | 3733 | GACCAGA CUGAUGAG X CGAA AGAGGUC | GACCUCU A UCUGGUC |
| 212 | HCV-3735 | 3735 | GUGACCA CUGAUGAG X CGAA AUAGAGG | CCUCUAU C UGGUCAC |
| 213 | HCV-3740 | 3740 | GUCUCGU CUGAUGAG X CGAA ACCAGAU | AUCUGGU C ACGAGAC |
| 214 | HCV-3761 | 3761 | GCACCGG CUGAUGAG X CGAA AUGACGU | ACGUCAU U CCGGUGC |
| 215 | HCV-3762 | 3762 | CGCACCG CUGAUGAG X CGAA AAUGACG | CGUCAUU C CGGUGCG |
| 216 | HCV-3786 | 3786 | CUCCCCC CUGAUGAG X CGAA ACCGUCA | UGACGGU C GGGGGAG |
| 217 | HCV-3797 | 3797 | GGGACAG CUGAUGAG X CGAA AGGCUCC | GGAGCCU A CUGUCCC |
| 218 | HCV-3802 | 3802 | UCUGGGG CUGAUGAG X CGAA ACAGUAG | CUACUGU C CCCCAGA |
| 219 | HCV-3835 | 3835 | GCCACCC CUGAUGAG X CGAA AAGAGCC | GGCUCUU C GGGUGGC |
| 220 | HCV-3851 | 3851 | AAGGGCA CUGAUGAG X CGAA AGGAGUG | CACUGCU C UGCCCUU |
| 221 | HCV-3858 | 3858 | UGCCCCG CUGAUGAG X CGAA AGGGCAG | CUGCCCU U CGGGGCA |
| 222 | HCV-3859 | 3859 | GUGCCCC CUGAUGAG X CGAA AAGGGCA | UGCCCUU C GGGGCAC |
| 223 | HCV-3872 | 3872 | AGAUGCC CUGAUGAG X CGAA ACAGCGU | ACGCUGU A GGCAUCU |
| 224 | HCV-3878 | 3878 | CCCGGAA CUGAUGAG X CGAA AUGCCUA | UAGGGAU C UUCCGGG |
| 225 | HCV-3880 | 3880 | AGCCCGG CUGAUGAG X CGAA AGAUGCC | GGCAUCU U CCGGGCU |
| 226 | HCV-3881 | 3881 | CAGCCCG CUGAUGAG X CGAA AAGAUGC | GCAUCUU C CGGGCUG |
| 227 | HCV-3908 | 3908 | CCUUCGC CUGAUGAG X CGAA ACCCCCC | GGGGGGU U GCGAAGG |
| 228 | HCV-4056 | 4056 | GGCACUU CUGAUGAG X CGAA AGUGCUC | GAGCACU A AAGUGCC |
| 229 | HCV-4072 | 4072 | GGCUGCG CUGAUGAG X CGAA ACGCAGC | GCUGCGU A CGCAGCC |
| 230 | HCV-4087 | 4087 | UACCUUG CUGAUGAG X CGAA ACCCUUG | CAAGGGU A CAAGGUA |
| 231 | HCV-4115 | 4115 | UGGCGGC CUGAUGAG X CGAA ACAGAUG | CAUCUGU U GCCGCCA |
| 232 | HCV-4175 | 4175 | CAGUUCU CUGAUGAG X CGAA AUGUUGG | CCAACAU C AGAACUG |
| 233 | HCV-4187 | 4187 | UGGUCCU CUGAUGAG X CGAA ACCCCAG | CUGGGGU A AGGACCA |
| 234 | HCV-4228 | 4228 | CUUACCA CUGAUGAG X CGAA AGGUGGA | UCCACCU A UGGUAAG |
| 235 | HCV-4233 | 4233 | AGGAACU CUGAUGAG X CGAA ACCAUAG | CUAUGGU A AGUUCCU |
| 236 | HCV-4237 | 4237 | GGCAAGG CUGAUGAG X CGAA ACUUACC | GGUAAGU U CCUUGCC |
| 237 | HCV-4238 | 4238 | CGGCAAG CUGAUGAG X CGAA AACUUAC | GUAAGUU C CUUGCCG |
| 238 | HCV-4241 | 4241 | CGUCGGC CUGAUGAG X CGAA AGGAACU | AGUUCCU U GCCGACG |
| 239 | HCV-4280 | 4280 | CACAUAU CUGAUGAG X CGAA AUGAUAU | AUAUCAU A AUAUGUG |
| 240 | HCV-4283 | 4283 | CAUCACA CUGAUGAG X CGAA AUUAUGA | UCAUAAU A UGUGAUG |
| 241 | HCV-4337 | 4337 | GGUCCAG CUGAUGAG X CGAA ACUGUGC | GCACAGU C CUGGACC |
| 242 | HCV-4370 | 4370 | GCACGAC CUGAUGAG X CGAA AGCCGCG | CGCGGCU C GUCGUGC |
| 243 | HCV-4373 | 4373 | CGAGCAC CUGAUGAG X CGAA ACGAGCC | GGCUCGU C GUGCUCG |
| 244 | HCV-4379 | 4379 | CGGUGGC CUGAUGAG X CGAA AGCACGA | UCGUGCU C GCCACCG |
| 245 | HCV-4425 | 4425 | UCCUCAA CUGAUGAG X CGAA AUUUGGG | CCCAAAU A UUGAGGA |
| 246 | HCV-4444 | 4444 | AGUGUUG CUGAUGAG X CGAA ACAGAGC | GCUCUGU C CAACACU |
| 247 | HCV-4460 | 4460 | AGAAGGG CUGAUGAG X CGAA AUCUCUC | GAGAGAU C CCCUUCU |
| 248 | HCV-4481 | 4481 | CGAGGGG CUGAUGAG X CGAA AUGGCCU | AGGCCAU C CCCCUCG |
| 249 | HCV-4487 | 4487 | UGGCCUC CUGAUGAG X CGAA AGGGGGA | UCCCCCU C GAGGCCA |
| 250 | HCV-4496 | 4496 | CCCCCUU CUGAUGAG X CGAA AUGGCCU | AGGCCAU C AAGGGGG |
| 251 | HCV-4528 | 4528 | CUUCUUG CUGAUGAG X CGAA AGUGGCA | UGCCACU C CAAGAAG |
| 252 | HCV-4577 | 4577 | CGGCAUU CUGAUGAG X CGAA AUUCCGA | UCGGAAU C AAUGCCG |
| 253 | HCV-4586 | 4586 | AAUACGC CUGAUGAG X CGAA ACGGCAU | AUGCCGU A GCGUAUU |
| 254 | HCV-4591 | 4591 | CCGGUAA CUGAUGAG X CGAA ACGCUAC | GUAGCGU A UUACCGG |
| 255 | HCV-4593 | 4593 | CCCCGGU CUGAUGAG X CGAA AUACGCU | AGCGUAU U ACCGGGG |
| 256 | HCV-4594 | 4594 | ACCCCGG CUGAUGAG X CGAA AAUACGC | GCGUAUU A CCGGGGU |
| 257 | HCV-4616 | 4616 | UCGGUAU CUGAUGAG X CGAA ACGGACA | UGUCCGU C AUACCGA |
| 258 | HCV-4619 | 4619 | UAGUCGG CUGAUGAG X CGAA AUGACGG | CCGUCAU A CCGACUA |
| 259 | HCV-4626 | 4626 | UCUCCGC CUGAUGAG X CGAA AGUCGGU | ACCGACU A GCGGAGA |
| 260 | HCV-4672 | 4672 | ACCGGUG CUGAUGAG X CGAA AGCCCGU | ACGGGCU A CACCGGU |
| 261 | HCV-4697 | 4697 | UGCAGUC CUGAUGAG X CGAA AUCACCG | CGGUGAU C GACUGCA |
| 262 | HCV-4789 | 4789 | UGAGCGC CUGAUGAG X CGAA ACACCGC | GCGGUGU C GCGCUCA |
| 263 | HCV-4795 | 4795 | CCGUUGU CUGAUGAG X CGAA AGCGCGA | UCGCGCU C ACAACGG |
| 264 | HCV-4920 | 4920 | UCAUACC CUGAUGAG X CGAA AGCACAG | CUGUGCU U GGUAUGA |
| 265 | HCV-4924 | 4924 | GAGCUCA CUGAUGAG X CGAA ACCAAGC | GCUUGGU A UGAGCUC |
| 266 | HCV-4931 | 4931 | CGGGCGU CUGAUGAG X CGAA AGCUCAU | AUGAGCU C ACGCCCG |
| 267 | HCV-4947 | 4947 | CUGACUG CUGAUGAG X CGAA AGUCUCA | UGAGACU A CAGUCAG |
| 268 | HCV-4952 | 4952 | GCAACCU CUGAUGAG X CGAA ACUGUAG | CUACAGU C AGGUUGC |
| 269 | HCV-4957 | 4957 | AGCCCGC CUGAUGAG X CGAA ACCUGAC | GUCAGGU U GCGGGCU |
| 270 | HCV-4965 | 4965 | UUCAGGU CUGAUGAG X CGAA AGCCCGC | GCGGGCU U ACCUGAA |
| 271 | HCV-4966 | 4966 | AUUCAGG CUGAUGAG X CGAA AAGCCCG | CGGGCUU A CCUGAAU |
| 272 | HCV-4974 | 4974 | CCUGGUG CUGAUGAG X CGAA AUUCAGG | CCUGAAU A CACCAGG |
| 273 | HCV-4984 | 4984 | GACGGGC CUGAUGAG X CGAA ACCCUGG | CCAGGGU U GCCCGUC |
| 274 | HCV-4991 | 4991 | CCUGGCA CUGAUGAG X CGAA ACGGGCA | UGCCCGU C UGGCAGG |
| 275 | HCV-5004 | 5004 | AACUCCA CUGAUGAG X CGAA AUGGUCC | GGACCAU C UGGAGUU |
| 276 | HCV-5102 | 5102 | GGUAUGC CUGAUGAG X CGAA ACCAGGU | ACCUGGU A GCAUACC |
| 277 | HCV-5107 | 5107 | GGCUUGG CUGAUGAG X CGAA AUGGUAC | GUAGCAU A CCAAGCC |
| 278 | HCV-5133 | 5133 | GGAGCCU CUGAUGAG X CGAA AGCCCUG | CAGGGCU C AGGCUCC |
| 279 | HCV-5218 | 5218 | UAGCCUA CUGAUGAG X CGAA ACAGGAG | CUGCUGU A UAGGCUA |
| 280 | HCV-5220 | 5220 | CCUAGCC CUGAUGAG X CGAA AUACAGC | GCUGUAU A GGCUAGG |
| 281 | HCV-5306 | 5306 | UAGUGAC CUGAUGAG X CGAA ACCUCCA | UGGAGGU C GUCACUA |
| 282 | HCV-5309 | 5309 | UGCUAGU CUGAUGAG X CGAA ACGACCU | AGGUCGU C ACUAGCA |
| 283 | HCV-5313 | 5313 | CAGGUGG CUGAUGAG X CGAA AGUGACG | CGUCACU A GCACCUG |
| 284 | HCV-5330 | 5330 | CUCCGCC CUGAUGAG X CGAA ACCAGGA | UGCUGGU A GGCGGAG |
| 285 | HCV-5339 | 5339 | CUGCAAG CUGAUGAG X CGAA ACUCCGC | GCGGAGU C CUUGCAG |
| 286 | HCV-5342 | 5342 | GAGCUGC CUGAUGAG X CGAA AGGACUC | GAGUCCU U GCAGCUC |
| 287 | HCV-5359 | 5359 | CAGGCAA CUGAUGAG X CGAA AUGCGGC | GCCGCAU A UUGCCUG |
| 288 | HCV-5361 | 5361 | GUCAGGC CUGAUGAG X CGAA AUAUGCG | CGCAUAU U GCCUGAC |
| 289 | HCV-5376 | 5376 | ACCACAC CUGAUGAG X CGAA ACCGGUU | AACCGGU A GUGUGGU |
| 290 | HCV-5399 | 5399 | ACAAAAU CUGAUGAG X CGAA AUCCUAC | GUAGGAU C AUUUUGU |
| 291 | HCV-5423 | 5423 | CGGGAAC CUGAUGAG X CGAA ACAGCCG | CGGCUGU U GUUCCCG |
| 292 | HCV-5426 | 5426 | UGUCGGG CUGAUGAG X CGAA ACAACAG | CUGUUGU U CCCGACA |
| 293 | HCV-5427 | 5427 | CUGUCGG CUGAUGAG X CGAA AACAACA | UGUUGUU C CCGACAG |
| 294 | HCV-5524 | 5524 | CUGCUUG CUGAUGAG X CGAA ACUCCUC | GAGCAGU U CAAGCAG |
| 295 | HCV-5525 | 5525 | UCUGCUU CUGAUGAG X CGAA AACUGCU | AGCAGUU C AAGCAGA |
| 296 | HCV-5583 | 5583 | ACCACGG CUGAUGAG X CGAA AGCAGCG | CGCUGCU C CCGUGGU |
| 297 | HCV-5596 | 5596 | CCACCUG CUGAUGAG X CGAA ACUCCAC | GUGGAGU C CAGGUGG |
| 298 | HCV-5612 | 5612 | AGGCCUC CUGAUGAG X CGAA AGGGCCC | GGGCCCU U GAGGCCU |
| 299 | HCV-5620 | 5620 | UGCCCAG CUGAUGAG X CGAA AGGCCUC | GAGGCCU U CUGGGCA |
| 300 | HCV-5621 | 5621 | UUGCCCA CUGAUGAG X CGAA AAGGCCU | AGGCCUU C UGGGCAA |
| 301 | HCV-5674 | 5674 | AGUGGAU CUGAUGAG X CGAA AGCCUGC | GCAGGCU U AUCCACU |
| 302 | HCV-5675 | 5675 | GAGUGGA CUGAUGAG X CGAA AAGCCUG | CAGGCUU A UCCACUC |
| 303 | HCV-5767 | 5767 | GAUGUUG CUGAUGAG X CGAA ACAGGAG | CUCCUGU U CAACAUC |
| 304 | HCV-5768 | 5768 | AGAUGUU CUGAUGAG X CGAA AACAGGA | UCCUGUU C AACAUCU |
| 305 | HCV-5801 | 5801 | GAGGAGC CUGAUGAG X CGAA AGUUGAG | CUCAACU C GCUCCUC |
| 306 | HCV-5805 | 5805 | CUGGGAG CUGAUGAG X CGAA AGCGAGU | ACUCGCU C CUCCCAG |
| 307 | HCV-5821 | 5821 | GAAGGCC CUGAUGAG X CGAA AAGCAGC | GCUGCUU C GGCCUUC |
| 308 | HCV-5827 | 5827 | GCCCACG CUGAUGAG X CGAA AGGCCGA | UCGGCCU U CGUGGGC |
| 309 | HCV-5828 | 5828 | CGGCCAC CUGAUGAG X CGAA AAGGCCG | CGGCCUU C GUGGGCG |
| 310 | HCV-5843 | 5843 | CACCGGC CUGAUGAG X CGAA AUGCCGG | CCGGCAU U GCCGGUG |
| 311 | HCV-5858 | 5858 | UGCUGCC CUGAUGAG X CGAA AUGGCCG | CGGCCAU U GGCAGGA |
| 312 | HCV-5867 | 5867 | CAAGGCC CUGAUGAG X CGAA AUGCUGC | GCAGCAU A GGCCUUG |
| 313 | HCV-5873 | 5873 | CCUUCCC CUGAUGAG X CGAA AGGCCUA | UAGGCCU U GGGAAGG |
| 314 | HCV-5905 | 5905 | CGCUCCA CUGAUGAG X CGAA AGCCCGG | GGGGGCU A UGGAGCG |
| 315 | HCV-5930 | 5930 | AAGCCAC CUGAUGAG X CGAA AGUGGAC | GUGCACU C GUGGCUU |
| 316 | HCV-5937 | 5937 | ACCUUAA CUGAUGAG X CGAA AGGCACG | CGUGGCU U UUAAGGU |
| 317 | HCV-5938 | 5938 | GACCUUA CUGAUGAG X CGAA AAGCCAC | GUGGCUU U UAAGGUC |
| 318 | HCV-5939 | 5939 | UGACCUU CUGAUGAG X CGAA AAAGCCA | UGGCUUU U AAGGUCA |
| 319 | HCV-5940 | 5940 | AUGACCU CUGAUGAG X CGAA AAAAGCC | GGCUUUU A AGGUCAU |
| 320 | HCV-5945 | 5945 | CGCUCAU CUGAUGAG X CGAA ACCUUAA | UUAAGGU C AUGAGCG |
| 321 | HCV-5965 | 5965 | CUCGGCG CUGAUGAG X CGAA AGGGCGC | GCGCCCU C CGCCGAG |
| 322 | HCV-5981 | 5981 | GCAAGUU CUGAUGAG X CGAA ACCAGGU | ACCUGGU U AACUUGC |
| 323 | HCV-5982 | 5982 | AGCAAGU CUGAUGAG X CGAA AACCAGG | CCUGGUU A ACUUGCU |
| 324 | HCv-5990 | 5990 | UGGCAGG CUGAUGAG X CGAA AGCAAGU | ACUUGCU C CCUGCCA |
| 325 | HCV-6004 | 6004 | GCCGGGG CUGAUGAG X CGAA AGAGGAU | AUCCUCU C CCCCGGC |
| 326 | HCV-6020 | 6020 | CCCCGAC CUGAUGAG X CGAA ACCAGGG | CCCUGGU C GUCGGGG |
| 327 | HCV-6023 | 6023 | CGACCCC CUGAUGAG X CGAA ACGACCA | UGGUCGU C GGGGUCG |
| 328 | HCV-6029 | 6029 | CACACAC CUGAUGAG X CGAA ACCCCGA | UCGGGGU C GUGUGUG |
| 329 | HCV-6044 | 6044 | GACGCAG CUGAUGAG X CGAA AUUGCUG | CAGCAAU C CUGCGUC |
| 330 | HCV-6051 | 6051 | ACGUGCC CUGAUGAG X CGAA ACGCAGG | CCUGCGU C GGCACGU |
| 331 | HCV-6106 | 6106 | CGAAGCG CUGAUGAG X CGAA ACGCUAU | AUAGCGU U CGCUUCG |
| 332 | HCV-6107 | 6107 | GCGAAGC CUGAUGAG X CGAA AACGCUA | UAGCGUU C GCUUCGC |
| 333 | HCV-6111 | 6111 | CCCCGCG CUGAUGAG X CGAA AGCGAAC | GUUCGCU U CGCGGGG |
| 334 | HCV-6413 | 6413 | UUUGCAU CUGAUGAG X CGAA AUGCCGU | ACGGCAU C AUGCAAA |
| 335 | HCV-6574 | 6574 | CCUGGAA CUGAUGAG X CGAA AGUUCGG | CCGAACU A UUCCAGG |
| 336 | HCV-6576 | 6576 | GCCCUGG CUGAUGAG X CGAA AUAGUUC | GAACUAU U CCAGGGC |
| 337 | HCV-6577 | 6577 | CGCCCUG CUGAUGAG X CGAA AAUAGUU | AACUAUU C CAGGGCG |
| 338 | HCV-6637 | 6637 | GUAGUGG CUGAUGAG X CGAA AGUCCCC | GGGGACU U CCACUAC |
| 339 | HCV-6638 | 6638 | CGUAGUG CUGAUGAG X CGAA AAGUCCC | GGGACUU C CACUACG |
| 340 | HCV-6643 | 6643 | CGUCACG CUGAUGAG X CGAA AGUGGAA | UUCCACU A CGUGACG |
| 341 | HCV-6671 | 6671 | GGCAUUU CUGAUGAG X CGAA ACGUUGU | ACAACGU A AAAUGCC |
| 342 | HCV-6703 | 6703 | GGUGAAG CUGAUGAG X CGAA AUUCCGG | CCCGAAU U CUUCACC |
| 343 | HCV-6704 | 6704 | CGGUGAA CUGAUGAG X CGAA AAUUCGG | CCGAAUU C UUCACCG |
| 344 | HCV-6706 | 6706 | UUCGGUG CUGAUGAG X CGAA AGAAUUC | GAAUUCU U CACCGAA |
| 345 | HCV-6707 | 6707 | AUUCGGU CUGAUGAG X CGAA AAGAAUU | AAUUCUU C ACCGAAU |
| 346 | HCV-6715 | 6715 | CCCGUCC CUGAUGAG X CGAA AUUCGGU | ACCGAAU U GGACGGG |
| 347 | HCV-6730 | 6730 | CCUGUGC CUGAUGAG X CGAA ACCGCAC | GUGCCGU U GCACAGG |
| 348 | HCV-6739 | 6739 | CGGAGCG CUGAUGAG X CGAA ACCUGUG | CACAGGU A CGCUCCG |
| 349 | HCV-6744 | 6744 | CACGCCG CUGAUGAG X CGAA AGCGUAC | GUACGCU C CGGCGUG |
| 350 | HCV-6759 | 6759 | CGUAGGA CUGAUGAG X CGAA AGGUCUG | CAGACCU C UCCUACG |
| 351 | HCV-6761 | 6761 | CCCGUAG CUGAUGAG X CGAA AGAGGUC | GACCUCU C CUACGGG |
| 352 | HCV-6764 | 6764 | CCUCCCG CUGAUGAG X CGAA AGGAGAG | CUCUCCU A CGGGAGG |
| 353 | HCV-6776 | 6776 | GGAAUGU CUGAUGAG X CGAA ACAUCCU | AGGAUGU C ACAUUCC |
| 354 | HCV-6782 | 6782 | CGACCUG CUGAUGAG X CGAA AAUGUGA | UCACAUU C CAGGUCG |
| 355 | HCV-6788 | 6788 | UGACCGC CUGAUGAG X CGAA ACCUGGA | UCCAGGU C GGGCUCA |
| 356 | HCV-6794 | 6794 | AUUGGUU CUGAUGAG X CGAA AGCCCGA | UCGGGCU C AACCAAU |
| 357 | HCV-6802 | 6802 | AACCAGG CUGAUGAG X CGAA AUUGGUU | AACCAAU A CCUGGUU |
| 358 | HCV-6809 | 6809 | GUGACCC CUGAUGAG X CGAA ACCAGGU | ACCUGGU U GGGUCAC |
| 359 | HCV-6814 | 6814 | GAGCUGU CUGAUGAG X CGAA ACCCAAC | GUUGGGU C ACAGCUC |
| 360 | HCV-6821 | 6821 | CGCAUGG CUGAUGAG X CGAA AGCUGUG | CACAGCU C CCAUGCG |
| 361 | HCV-6906 | 6906 | GCCAGCC CUGAUGAG X CGAA ACGUUUA | UAAACGU A GGCUGGC |
| 362 | HCV-6922 | 6922 | GGGGGGA CUGAUGAG X CGAA ACCCCCU | AGGGGGU C UCCCCCC |
| 363 | HCV-6924 | 6924 | GAGGGGG CUGAUGAG X CGAA AGACCCC | GGGGUCU C CCCCCUC |
| 364 | HCV-6931 | 6931 | GGCCAAG CUGAUGAG X CGAA AGGGGGG | CCCCCCU C CUUGGCC |
| 365 | HCV-6934 | 6934 | GCUGGCC CUGAUGAG X CGAA AGGAGGG | CCCUCCU U GGCCAGC |
| 366 | HCV-6943 | 6943 | AGCUGAA CUGAUGAG X CGAA AGCUCGC | GCCAGCU C UUCAGCU |
| 367 | HCV-6958 | 6953 | CGCAGAC CUGAUGAG X CGAA AUUGGCU | AGCCAAU U GUCUGCG |
| 368 | HCV-6961 | 6961 | AGGCGCA CUGAUGAG X CGAA ACAAUUG | CAAUUGU C UGCGCCU |
| 369 | HCV-7034 | 7034 | GCCACAG CUGAUGAG X CGAA AGGUUGG | CCAACCU C CUGUGGC |
| 370 | HCV-7118 | 7118 | CCGCUCG CUGAUGAG X CGAA AGCGGGU | ACCCGCU U CGAGCGG |
| 371 | HCV-7119 | 7119 | UCCGCUC CUGAUGAG X CGAA AAGCGGG | CCCGCUU C GAGGGGA |
| 372 | HCV-7145 | 7145 | CAACGGA CUGAUGAG X CGAA ACUUCCC | GGGAAGU A UCCGUUG |
| 373 | HCV-7195 | 7195 | UAUGGGC CUGAUGAG X CGAA ACGCGGG | CCCGCGU U GCCCAUA |
| 374 | HCV-7202 | 7202 | GUGCCCA CUGAUGAG X CGAA AUGGGCA | UGCCCAU A UGGGCAC |
| 375 | HCV-7218 | 7218 | GGGUUGU CUGAUGAG X CGAA AUCCGGG | CCCGGAU U ACAACCC |
| 376 | HCV-7219 | 7219 | AGGGUUG CUGAUGAG X CGAA AAUCCGG | CCGGAUU A CAACCCU |
| 377 | HCV-7234 | 7234 | GGACUCU CUGAUGAG X CGAA ACAGUGG | CCACUGU U AGAGUCC |
| 378 | HCV-7235 | 7235 | AGGACUC CUGAUGAG X CGAA AACAGUG | CACUGUU A GAGUCCU |
| 379 | HCV-7251 | 7251 | UAGUCCG CUGAUGAG X CGAA ACUUUUC | GAAAAGU C CGGACUA |
| 380 | HCV-7258 | 7258 | AGGGACG CUGAUGAG X CGAA AGUCCGG | CCGGACU A CGUCCCU |
| 381 | HCV-7262 | 7262 | CCGGAGG CUGAUGAG X CGAA ACGUAGU | ACUACGU C CCUCCGG |
| 382 | HCV-7266 | 7266 | ACCGCCG CUGAUGAG X CGAA AGGGACG | CGUCCCU C CGGCGGU |
| 383 | ECV-7288 | 7288 | AGGCGGC CUGAUGAG X CGAA AUGGGCA | UGCCCAU U GCCGCCU |
| 384 | HCV-7296 | 7296 | CCCGUGG CUGAUGAG X CGAA AGGCGGC | GCCGCCU A CCACGGG |
| 385 | HCV-7354 | 7354 | CACGGUG CUGAUGAG X CGAA ACUCUGU | ACAGAGU C CACCGUG |
| 386 | HCV-7386 | 7386 | GUCUUAG CUGAUGAG X CGAA AGCCAGC | GCUGGCU A CUAAGAC |
| 387 | HCV-7389 | 7389 | AAAGUCU CUGAUGAG X CGAA AGUAGCC | GGCUACU A AGACUUU |
| 388 | HCV-7395 | 7395 | CUGCCGA CUGAUGAG X CGAA AGUCUUA | UAAGACU U UCGGCAG |
| 389 | HCV-7396 | 7396 | GCUGCCG CUGAUGAG X CGAA AAGUCUU | AAGACUU U CGGCAGC |
| 390 | HCV-7397 | 7397 | AGCUGCC CUGAUGAG X CGAA AAAGUCU | AGACUUU C GGCAGCU |
| 391 | HCV-7411 | 7411 | GGCCGAC CUGAUGAG X CGAA AUCCGGA | UCCGGAU C GUCGGCC |
| 392 | HCV-7414 | 7414 | AACGGCC CUGAUGAG X CGAA ACGAUCC | GGAUCGU C GGCCGUU |
| 393 | HCV-7421 | 7421 | CGCUGUC CUGAUGAG X CGAA ACGGCCG | CGGCCGU U GACAGCG |
| 394 | HCV-7498 | 7498 | CAUGGAG CUGAUGAG X CGAA AGUACGA | UCGUACU C CUCCAUG |
| 395 | HCV-7501 | 7501 | GGGCAUG CUGAUGAG X CGAA AGGAGUA | UACUCCU C CAUGCCC |
| 396 | HCV-7514 | 7514 | CCCCCUC CUGAUGAG X CGAA AGGGGGG | CCCCCCU U GAGGGGG |
| 397 | HCV-7539 | 7539 | UCGCUGA CUGAUGAG X CGAA AUCAGGG | CCCUGAU C UCAGCGA |
| 398 | HCV-7541 | 7541 | CGUCGCU CUGAUGAG X CGAA AGAUCAG | CUGAUCU C AGCGACG |
| 399 | HCV-7552 | 7552 | AGACCAA CUGAUGAG X CGAA ACCCGUC | GACGGGU C UUGGUCU |
| 400 | HCV-7554 | 7554 | GUAGACC CUGAUGAG X CGAA AGACCCG | CGGGUCU U GGUCUAC |
| 401 | HCV-7558 | 7558 | CACGGUA CUGAUGAG X CGAA ACCAAGA | UCUUGGU C UACCGUG |
| 402 | HCV-7560 | 7560 | CUCACGG CUGAUGAG X CGAA AGACCAA | UUGGUCU A CCGUGAG |
| 403 | HCV-7589 | 7589 | AGCAGAC CUGAUGAG X CGAA AUGUCGU | ACGACAU C GUCUGCU |
| 404 | HCV-7592 | 7592 | AGCAGCA CUGAUGAG X CGAA ACGAUGU | ACAUCGU C UGCUGCU |
| 405 | HCV-7600 | 7600 | GGACAUU CUGAUGAG X CGAA AGCAGCA | UGCUGCU C AAUGUCC |
| 406 | HCV-7606 | 7606 | UGUGUAG CUGAUGAG X CGAA ACAUUGA | UCAAUGU C CUACACA |
| 407 | HCV-7667 | 7667 | ACGCGUU CUGAUGAG X CGAA AUGGGCA | UGCCCAU C AACGCGU |
| 408 | HCV-7723 | 7723 | ACUGCGG CUGAUGAG X CGAA AUGUUGU | ACAACAU C CCGCAGU |
| 409 | HCV-7775 | 7775 | CGUCCAG CUGAUGAG X CGAA ACUUGCA | UGCAAGU C CUGGACG |
| 410 | HCV-7789 | 7789 | GUCCCGG CUGAUGAG X CGAA AGUGGUC | GACCACU A CCGGGAC |
| 411 | HCV-7839 | 7839 | AGAAGUU CUGAUGAG X CGAA AGCCUUA | UAAGGCU A AACUUCU |
| 412 | HCV-7847 | 7847 | CUACGGA CUGAUGAG X CGAA AGAAGUU | AACUUCU A UCCGUAG |
| 413 | HCV-7849 | 7849 | UUCUACG CUGAUGAG X CGAA AUAGAAG | CUUCUAU C CGUAGAA |
| 414 | HCV-7853 | 7853 | CUUCUUC CUGAUGAG X CGAA ACGGAUA | UAUCCGU A GAAGAAG |
| 415 | HCV-7894 | 7894 | AAAUUUA CUGAUGAG X CGAA AUUUGGC | GCCAAAU C UAAAUUU |
| 416 | HCV-7896 | 7896 | CCAAAUU CUGAUGAG X CGAA AGAUUUG | CAAAUCU A AAUUUGG |
| 417 | HCV-7900 | 7900 | AUAGCCA CUGAUGAG X CGAA AUUUAGA | UCUAAAU U UGGCUAU |
| 418 | HCV-7901 | 7901 | CAUAGCC CUGAUGAG X CGAA AAUUUAG | CUAAAUU U GGCUAUG |
| 419 | HCV-7906 | 7906 | UGCCCCA CUGAUGAG X CGAA AGCCAAA | UUUGGCU A UGGGGCA |
| 420 | HCV-7955 | 7955 | CGGAGCG CUGAUGAG X CGAA AUGUGGU | ACCACAU C CGCUCCG |
| 421 | HCV-7960 | 7960 | CCACACG CUGAUGAG X CGAA AGCGGAU | AUCCGCU C CGUGUGG |
| 422 | HCV-8075 | 8075 | AUACGAU CUGAUGAG X CGAA AGGCGAG | CUCGCCU U AUCGUAU |
| 423 | HCV-8076 | 8076 | AAUACGA CUGAUGAG X CGAA AAGGCGA | UCGCCUU A UCGUAUU |
| 424 | HCV-8078 | 8078 | GGAAUAC CUGAUGAG X CGAA AUAAGGC | GCCUUAU C GUAUUCC |
| 425 | HCV-8170 | 8170 | GAAUCCG CUGAUGAG X CGAA ACGAGGA | UCCUCGU A CGGAUUC |
| 426 | HCV-8176 | 8176 | GUACUGG CUGAUGAG X CGAA AUCCGUA | UACGGAU U CCAGUAC |
| 427 | HCV-8182 | 8182 | AGGAGAG CUGAUGAG X CGAA ACUGGAA | UUCCAGU A CUCUCCU |
| 428 | ECV-8187 | 8187 | UGCCCAG CUGAUGAG X CGAA AGAGUAC | GUACUCU C CUGGGCA |
| 429 | HCV-8201 | 8201 | GGAACUC CUGAUGAG X CGAA ACCCGCU | AGCGGGU U GAGUUCC |
| 430 | HCV-8206 | 8206 | CACCAGG CUGAUGAG X CGAA ACUCAAC | GUUGAGU U CCUGGUG |
| 431 | HCV-8207 | 8207 | UCACCAG CUGAUGAG X CGAA AACUCAA | UUGAGUU C CUGGUGA |
| 432 | HCV-8227 | 8227 | UUUCUUU CUGAUGAG X CGAA AUUUCCA | UGGAAAU C AAAGAAA |
| 433 | HCV-8357 | 8357 | GCGACUU CUGAUGAG X CGAA AUGGCCU | AGGCCAU A AAGUCGC |
| 434 | HCV-8362 | 8362 | CGUGAGC CUGAUGAG X CGAA ACUUUAU | AUAAAGU C GCUCACG |
| 435 | HCV-8366 | 8366 | GCUCCGU CUGAUGAG X CGAA AGCGACU | AGUCGCU C ACGGAGC |
| 436 | HCV-8378 | 8378 | CGAUGUA CUGAUGAG X CGAA AGCCGCU | AGCGGCU C UACAUCG |
| 437 | HCV-8380 | 8380 | CCCGAUG CUGAUGAG X CGAA AGAGCCG | CGGCUCU A CAUCGGG |
| 438 | HCV-8384 | 8384 | GGCCCCC CUGAUGAG X CGAA AUGUAGA | UCUACAU C GGGGGCC |
| 439 | HCV-8424 | 8424 | CGGCGAU CUGAUGAG X CGAA ACCGCAG | CUGCGGU U AUCGCCG |
| 440 | HCV-8425 | 8425 | CCGGCGA CUGAUGAG X CGAA AACCGCA | UGCGGUU A UCGCCGG |
| 441 | HCV-8427 | 8427 | CACCGGC CUGAUGAG X CGAA AUAACCG | CGGUUAU C GCCGGUG |
| 442 | HCV-8460 | 8460 | CCGCAGC CUGAUGAG X CGAA AGUCGUC | GACGACU A GCUGCGG |
| 443 | HCV-8508 | 8508 | GCAGCUC CUGAUGAG X CGAA ACAGGCC | GGCCUGU C GAGCUGC |
| 444 | HCV-8522 | 8522 | AGUCCUG CUGAUGAG X CGAA AGCUUUG | CAAAGCU C CAGGACU |
| 445 | HCV-8540 | 8540 | CGUUCAC CUGAUGAG X CGAA AGCAUCG | CGAUGCU C GUGAACG |
| 446 | HCV-8558 | 8558 | UAACGAC CUGAUGAG X CGAA AGGUCGU | ACGACCU U GUCGUUA |
| 447 | HCV-8561 | 8561 | AGAUAAC CUGAUGAG X CGAA ACAAGGU | ACCUUGU C GUUAUCU |
| 448 | HCV-8564 | 8564 | CACAGAU CUGAUGAG X CGAA ACGACAA | UUGUCGU U AUCUGUG |
| 449 | HCV-8638 | 8638 | GGGGGCA CUGAUGAG X CGAA AGUACCU | AGGUACU C UGCCCCC |
| 450 | HCV-8671 | 8671 | CAAGUCG CUGAUGAG X CGAA AUUCUGG | CCAGAAU A CGACUUG |
| 451 | HCV-8698 | 8698 | GUUGGAG CUGAUGAG X CGAA AGCAUGA | UCAUGCU C CUCCAAC |
| 452 | HCV-8701 | 8701 | CACGUUG CUGAUGAG X CGAA AGGAGCA | UGCUCCU C CAACGUG |
| 453 | HCV-8728 | 8728 | UUUGCCG CUGAUGAG X CGAA AUGCGUC | GACGCAU C CGGCAAA |
| 454 | HCV-8774 | 8774 | CCCGUGC CUGAUGAG X CGAA AGGGGGG | CCCCCCU U GCACGGG |
| 455 | HCV-8842 | 8842 | GGGCGCA CUGAUGAG X CGAA ACAUGAU | AUCAUGU A UGCGCCC |
| 456 | HCV-8854 | 8854 | UGCCCAU CUGAUGAG X CGAA AGGUGGG | CCCACCU U AUGGGCA |
| 457 | HCV-8855 | 8855 | UUGCCCA CUGAUGAG X CGAA AAGGUGG | CCACCUU A UGGGCAA |
| 458 | HCV-8871 | 8871 | GUCAUCA CUGAUGAG X CGAA AAUCAUC | GAUGAUU U UGAUGAC |
| 459 | HCU-8880 | 8880 | AAGAAGU CUGAUGAG X CGAA AGUCAUC | GAUGACU C ACUUCUU |
| 460 | HCU-8931 | 8931 | AUCUGAC CUGAUGAG X CGAA AUCCAGG | CCUGGAU U GUCAGAU |
| 461 | HCV-8934 | 8934 | UAGAUCU CUGAUGAG X CGAA ACAAUCC | GGAUUGU C AGAUCUA |
| 462 | HCU-8939 | 8939 | CCCCGUA CUGAUGAG X CGAA AUCUGAC | GUCAGAU C UACGGGG |
| 463 | HCV-8941 | 8941 | GGCCCCG CUGAUGAG X CGAA AGAUCUG | CAGAUCU A CGGGGCC |
| 464 | HCV-9065 | 9065 | GUUUCCU CUGAUGAG X CGAA AGGCAUG | CAUGCCU C AGGAAAC |
| 465 | HCV-9074 | 9074 | GUACCCC CUGAUGAG X CGAA AGUUUCC | GGAAACU U GGGGUAC |
| 466 | HCV-9080 | 9080 | AGGGCGG CUGAUGAG X CGAA ACCCCAA | UUGGGGU A CCGCCCU |
| 467 | HCV-9088 | 9088 | GACUCGC CUGAUGAG X CGAA AGGGCGG | CCGCCCU U GCGAGUC |
| 468 | HCV-9095 | 9095 | GUCUCCA CUGAUGAG X CGAA ACUCGCA | UGCGAGU C UGGAGAC |
| 469 | HCV-9119 | 9119 | UAGCGCG CUGAUGAG X CGAA ACACUUC | GAAGUGU C CGCGCUA |
| 470 | HCV-9126 | 9126 | AGUAGCC CUGAUGAG X CGAA AGCGCGG | CCGCGCU A GGCUACU |
| 471 | HCV-9131 | 9131 | GGGACAG CUGAUGAG X CGAA AGCCUAG | CUAGGCU A CUGUCCC |
| 472 | HCV-9136 | 9136 | CCCUUGG CUGAUGAG X CGAA ACAGUAG | CUACUGU C CCAAGGG |
| 473 | HCV-9226 | 9226 | CAGCUGG CUGAUGAG X CGAA ACGCGGC | GCCGCGU C CCAGCUG |
| 474 | HCV-9238 | 9238 | GCUGGAC CUGAUGAG X CGAA AGUCCAG | CUGGACU U GUCCAGC |
| 475 | HCV-9241 | 9241 | CCAGCUG CUGAUGAG X CGAA ACAAGUC | GACUUGU C CAGCUGG |
| 476 | HCV-9250 | 9250 | AGCAACG CUGAUGAG X CGAA ACCAGCU | AGCUGGU U CGUUGCU |
| 477 | HCV-9251 | 9251 | CAGCAAC CUGAUGAG X CGAA AACCAGC | GCUGGUU C GUUGCUG |
| 478 | HCU-9254 | 9254 | AACCAGC CUGAUGAG X CGAA ACGAACC | GGUUCGU U GCUGGUU |
| 479 | HCV-9278 | 9278 | UGUGAUA CUGAUGAG X CGAA AUGUCUC | GAGACAU A UAUCACA |
| 480 | HCV-9280 | 9280 | GCUGUGA CUGAUGAG X CGAA AUAUGUC | GACAUAU A UCACAGC |
| 481 | HCV-9282 | 9282 | AGGCUGU CUGAUGAG X CGAA AUAUAUG | CAUAUAU C ACAGCCU |
| 482 | HCV-9292 | 9292 | GGCACGA CUGAUGAG X CGAA ACAGGCU | AGCCUGU C UCGUGCC |
| 483 | HCV-9326 | 9326 | GUAGGAG CUGAUGAG X CGAA AGGCACC | GGUGCCU A CUCCUAC |
| 484 | HCV-9329 | 9329 | AAAGUAG CUGAUGAG X CGAA AGUAGGC | GCCUACU C CUACUUU |
| 485 | HCV-9332 | 9332 | CGGAAAG CUGAUGAG X CGAA AGGAGUA | UACUCCU A CUUUCCG |
| 486 | HCV-9335 | 9335 | CUACGGA CUGAUGAG X CGAA AGUAGGA | UCCUACU U UCCGUAG |
| 487 | HCV-9336 | 9336 | CCUACGG CUGAUGAG X CGAA AAGUAGG | CCUACUU U CCGUAGG |
| 488 | HCV-9337 | 9337 | CCCUACG CUGAUGAG X CGAA AAAGUAG | CUACUUU C CGUAGGG |
| 489 | HCV-9341 | 9341 | CUACCCC CUGAUGAG X CGAA ACGGAAA | UUUCCGU A GGGGUAG |
| 490 | HCV-9347 | 9347 | AGAUGCC CUGAUGAG X CGAA ACCCCUA | UAGGGGU A GGCAUCU |
| 491 | HCV-9353 | 9353 | GCAGGUA CUGAUGAG X CGAA AUGCCUA | UAGGCAU C UACCUGC |
| 492 | HCV-9355 | 9355 | GAGCAGG CUGAUGAG X CGAA AGAUGCC | GGCAUCU A CCUGCUC |
| 493 | HCV-9362 | 9362 | GGUUGGG CUGAUGAG X CGAA AGCAGGU | ACCUGCU C CCCAACC |
| 494 | HCV-9385 | 9385 | GAGUGAU CUGAUGAG X CGAA AGCUCCC | GGGAGCU A AUCACUC |
| 495 | HCV-9388 | 9388 | CUGGAGU CUGAUGAG X CGAA AUUAGCU | AGCUAAU C ACUCCAG |
| 496 | HCV-9392 | 9392 | UGGCCUG CUGAUGAG X CGAA AGUGAUU | AAUCACU C CAGGCCA |
| 497 | HCV-9402 | 9402 | GAUGGCC CUGAUGAG X CGAA AUUGGCC | GGCCAAU A GGCCAUC |

Where “X” represents stem II region of a HH ribozyme (Hertel et al., 1992 Nucleic Acids Res. 20: 3252). The length of stem II may be ≧2 base-pairs.

TABLE VI
Additional HCV Hammerhead (HH) Ribozyme and
Target Sequence

| | | |
|---|---|---|
| Pos. | Ribozyme | Substrate |

| | | |
|---|---|---|
| 14 | CGCCCCC CUGAUGAG X CGAA AUCGGGG | CCCCGAU U GGGGGCG |
| 34 | AGUGAUC CUGAUGAG X CGAA AUGGUGG | CCACCAU A GAUCACU |
| 38 | GGGGAGU CUGAUGAG X CGAA AUCUAUG | CAUAGAU C ACUCCCC |
| 42 | CACAGGG CUGAUGAG X CGAA AGUGAUC | GAUCACU C CCCUGUG |
| 57 | AAGACAG CUGAUGAG X CGAA AGUUCCU | AGGAACU A CUGUCUU |
| 62 | GCGUGAA CUGAUGAG X CGAA ACAGUAG | CUACUGU C UUCACGC |
| 64 | CUGCGUG CUGAUGAG X CGAA AGACAGU | ACUGUCU U CACGCAG |
| 65 | UCUGCGU CUGAUGAG X CGAA AAGACAG | CUGUCUU C ACGCAGA |
| 79 | AUGGCUA CUGAUGAG X CGAA ACGCUUU | AAAGCGU C UAGCCAU |
| 81 | CCAUGGC CUGAUGAG X CGAA AGACGCU | AGCGUCU A GCCAUGG |
| 92 | UCAUACU CUGAUGAG X CGAA ACGCCAU | AUGGCGU U AGUAUGA |
| 93 | CUCAUAC CUGAUGAG X CGAA AACGCCA | UGGCGUU A GUAUGAG |
| 96 | ACACUCA CUGAUGAG X CGAA ACUAACG | CGUUAGU A UGAGUGU |
| 104 | GCUGCAC CUGAUGAG X CGAA ACACUCA | UGAGUGU C GUGCAGC |
| 142 | AGACCAC CUGAUGAG X CGAA AUGGCUC | GAGCCAU A GUGGUCU |
| 192 | AAGAAAG CUGAUGAG X CGAA ACCCGGU | ACCGGGU C CUUUCUU |
| 195 | UCCAAGA CUGAUGAG X CGAA AGGACCC | GGGUCCU U UCUUGGA |
| 196 | AUCCAAG CUGAUGAG X CGAA AAGGACC | GGUCCUU U CUUGGAU |
| 197 | GAUCCAA CUGAUGAG X CGAA AAAGGAC | GUCCUUU C UUGGAUC |
| 204 | GCGGGUU CUGAUGAG X CGAA AUCCAAG | CUUGGAU C AACCCGC |
| 227 | ACGCCCA CUGAUGAG X CGAA AUCUCCA | UGGAGAU U UGGGCGU |
| 228 | CACGCCC CUGAUGAG X CGAA AAUCUCC | GGAGAUU U GGGCGUG |
| 282 | GUACCAC CUGAUGAG X CGAA AGGCCUU | AAGGCCU U GUGGUAC |
| 354 | GGUUUAG CUGAUGAG X CGAA AUUCGUG | CACGAAU C CUAAACC |
| 357 | UGAGGUU CUGAUGAG X CGAA AGGAUUC | GAAUCCU A AACCUCA |
| 363 | UUUCUUU CUGAUGAG X CGAA AGGUUUA | UAAACCU C AAAGAAA |
| 381 | UAGGUGU CUGAUGAG X CGAA ACGUUUG | CAAACGU A ACACCUA |
| 388 | GCGGCGG CUGAUGAG X CGAA AGGUGUU | AACACCU A CCGCCGC |
| 431 | CACCAAC CUGAUGAG X CGAA AUCUGAC | GUCAGAU C GUUGGUG |
| 434 | CUCCACC CUGAUGAG X CGAA ACGAUCU | AGAUCGU U GGUGGAG |
| 443 | ACACGUA CUGAUGAG X CGAA ACUCCAC | GUGGAGU U UACGUGU |
| 444 | AACACGU CUGAUGAG X CGAA AACUCCA | UGGAGUU U ACGUGUU |
| 445 | CAACACG CUGAUGAG X CGAA AAACUCC | GGAGUUU A CGUGUUG |
| 451 | GCGCGGC CUGAUGAG X CGAA ACACGUA | UACGUGU U GCCGCGC |
| 516 | CUUCCAC CUGAUGAG X CGAA AGGUUGC | GCAACCU C GUGGAAG |
| 688 | AUUGCGC CUGAUGAG X CGAA ACCUCCG | CGGAGGU C GCGCAAU |
| 702 | AUGACCU CUGAUGAG X CGAA ACCCAGA | UCUGGGU A AGGUCAU |
| 719 | CGCACGU CUGAUGAG X CGAA AGGGUAU | AUACCCU C ACGUGCG |
| 740 | ACCCCAU CUGAUGAG X CGAA AGGUCGG | CCGACCU C AUGGGGU |
| 861 | AUAGAGA CUGAUGAG X CGAA AGAGCAA | UUGCUCU U UCUCUAU |
| 862 | GAUAGAG CUGAUGAG X CGAA AAGAGCA | UGCUCUU U CUCUAUC |
| 863 | AGAUAGA CUGAUGAG X CGAA AAAGAGC | GCUCUUU C UCUAUCU |
| 865 | GAAGAUA CUGAUGAG X CGAA AGAAAGA | UCUUUCU C UAUCUUC |
| 867 | AGGAAGA CUGAUGAG X CGAA AGAGAAA | UUUCUCU A UCUUCCU |
| 869 | AGAGGAA CUGAUGAG X CGAA AUAGAGA | UCUCUAU C UUCCUCU |
| 871 | CAAGAGG CUGAUGAG X CGAA AGAUAGA | UCUAUCU U CCUCUUG |
| 872 | CCAAGAG CUGAUGAG X CGAA AAGAUAG | CUAUCUU C CUCUUGG |
| 875 | GGGCCAA CUGAUGAG X CGAA AGGAAGA | UCUUCCU C UUGGCCC |
| 877 | CAGGGCC CUGAUGAG X CGAA AGAGGAA | UUCCUCU U GGCCCUG |
| 889 | CAAACAG CUGAUGAG X CGAA ACAGCAG | CUGCUGU C CUGUUUG |
| 894 | AUGGUCA CUGAUGAG X CGAA ACAGGAC | GUCCUGU U UGACCAU |
| 895 | GAUGGUC CUGAUGAG X CGAA AACAGGA | UCCUGUU U GACCAUC |
| 902 | AAGCUGG CUGAUGAG X CGAA AUGGUCA | UGACCAU C CCAGCUU |
| 909 | UAAGCGG CUGAUGAG X CGAA AGCUGGG | CCCAGCU U CCGCUUA |
| 910 | AUAAGCG CUGAUGAG X CGAA AAGCUGG | CCAGCUU C CGCUUAU |
| 915 | ACCUGAU CUGAUGAG X CGAA AGCGGAA | UUCCGCU U AUCAGGU |
| 916 | CACCUGA CUGAUGAG X CGAA AAGCGGA | UCCGCUU A UCAGGUG |
| 918 | CGCACCU CUGAUGAG X CGAA AUAAGCG | CGCUUAU C AGGUGCG |
| 934 | CAGCCCG CUGAUGAG X CGAA AUGCGUU | AACGCAU C CGGGCUG |
| 943 | GACAUGG CUGAUGAG X CGAA ACAGCCC | GGGCUGU A CCAUGUC |
| 950 | CAUUCGU CUGAUGAG X CGAA ACAUGGU | ACCAUGU C ACGAAUG |
| 964 | UGAGUUG CUGAUGAG X CGAA AGCAGUC | GACUGCU C CAACUCA |
| 970 | AAUGCUU CUGAUGAG X CGAA AGUUGGA | UCCAACU C AAGCAUU |
| 977 | CAUACAC CUGAUGAG X CGAA AUGCUUG | CAAGCAU U GUGUAUG |
| 1008 | CCGGGGG CUGAUGAG X CGAA AUGCAUG | CAUGCAU A CCCCCGG |
| 1067 | UGGGAGU CUGAUGAG X CGAA AGCGCUA | UAGCGCU C ACUCCCA |
| 1071 | AGCGUGG CUGAUGAG X CGAA AGUGAGC | GCUCACU C CCACGCU |
| 1079 | UGGCCGC CUGAUGAG X CGAA AGCGUGG | CCACGCU C GCGGCCA |
| 1100 | UAGUGGG CUGAUGAG X CGAA AUGCUGG | CCAGCAU C CCCACUA |
| 1107 | AUUGUCG CUGAUGAG X CGAA AGUGGGG | CCCCACU A CGACAAU |
| 1115 | GGCGUCG CUGAUGAG X CGAA AUUGUCG | CGACAAU A CGACGCC |
| 1152 | GAACAGA CUGAUGAG X CGAA AGCGGCC | GGCCGCU U UCUGUUC |
| 1181 | AUCCGCA CUGAUGAG X CGAA AGGUCCC | GGGACCU C UGCGGAU |
| 1199 | GGGAGAC CUGAUGAG X CGAA AGGAAAA | UUUUCCU C GUCUCCC |
| 1202 | ACUGGGA CUGAUGAG X CGAA ACGAGGA | UCCUCGU C UCCCAGU |
| 1204 | CAACUGG CUGAUGAG X CGAA AGACGAG | CUCGUCU C CCAGUUG |
| 1210 | GGUGAAC CUGAUGAG X CGAA ACUGGGA | UCCCAGU U GUUCACC |
| 1213 | GAAGGUG CUGAUGAG X CGAA ACAACUG | CAGUUGU U CACCUUC |
| 1214 | AGAAGGU CUGAUGAG X CGAA AACAACU | AGUUGUU C ACCUUCU |
| 1219 | AGGCGAG CUGAUGAG X CGAA AGGUGAA | UUCACCU U CUCGCCU |
| 1220 | GAGGCGA CUGAUGAG X CGAA AAGGUGA | UCACCUU C UCGCCUC |
| 1222 | GCGAGGC CUGAUGAG X CGAA AGAAGGU | ACCUUCU C GCCUCGC |
| 1227 | UACCGGC CUGAUGAG X CGAA AGGCGAG | CUCGCCU C GCCGGUA |
| 1234 | UGUCUCA CUGAUGAG X CGAA ACCGGCG | CGCCGGU A UGAGACA |
| 1244 | AGUCCUG CUGAUGAG X CGAA ACUGUCU | AGACAGU A CAGGACU |
| 1257 | AUUGAGC CUGAUGAG X CGAA AUUGCAG | CUGCAAU U GCUCAAU |
| 1261 | AUAGAUU CUGAUGAG X CGAA AGCAAUU | AAUUGCU C AAUCUAU |
| 1265 | CGGGAUA CUGAUGAG X CGAA AUUGAGC | GCUCAAU C UAUCCCG |
| 1267 | GCCGGGA CUGAUGAG X CGAA AGAUUGA | UCAAUCU A UCCCGGC |
| 1269 | UGGCCGG CUGAUGAG X CGAA AUAGAUU | AAUCUAU C CCGGCCA |
| 1299 | AUAUCCC CUGAUGAG X CGAA AGCCAUG | CAUGGCU U GGGAUAU |
| 1305 | AUCAUCA CUGAUGAG X CGAA AUCCCAA | UUGGGAU A UGAUGAU |
| 1321 | UGUAGGC CUGAUGAG X CGAA ACCAGUU | AACUGGU C GCCUACA |
| 1326 | GCUGUUG CUGAUGAG X CGAA AGGCGAC | GUCGCCU A CAACAGC |
| 1337 | ACACCAC CUGAUGAG X CGAA AGGGCUG | CAGCCCU A GUGGUGU |
| 1345 | UAACUGC CUGAUGAG X CGAA ACACCAC | GUGGUGU C GCAGUUA |
| 1351 | CCGGAGU CUGAUGAG X CGAA ACUGCGA | UCGCAGU U ACUCCGG |
| 1352 | UCCGGAG CUGAUGAG X CGAA AACUGCG | CGCAGUU A CUCCGGA |
| 1355 | GGAUCCG CUGAUGAG X CGAA AGUAACU | AGUUACU C CGGAUCC |
| 1361 | CUUGUGG CUGAUGAG X CGAA AUCCGGA | UCCGGAU C CCACAAG |
| 1449 | AAGACCU CUGAUGAG X CGAA AGCCCAG | CUGGGCU A AGGUCUU |
| 1454 | CAAUCAA CUGAUGAG X CGAA ACCUUAG | CUAAGGU C UUGAUUG |
| 1456 | CACAAUC CUGAUGAG X CGAA AGACCUU | AAGGUCU U GAUUGUG |
| 1460 | ACAUCAC CUGAUGAG X CGAA AUCAAGA | UCUUGAU U GUGAUGU |
| 1468 | AAAGAGU CUGAUGAG X CGAA ACAUCAC | GUGAUGU U ACUCUUU |
| 1469 | CAAAGAG CUGAUGAG X CGAA AACAUCA | UGAUGUU A CUCUUUG |
| 1472 | CGGCAAA CUGAUGAG X CGAA AGUAACA | UGUUACU C UUUGCCG |
| 1474 | GCCGGCA CUGAUGAG X CGAA AGAGUAA | UUACUCU U UGCCGGC |
| 1475 | CGCCGGC CUGAUGAG X CGAA AAGAGUA | UACUCUU U GCCGGCG |
| 1484 | CCCCGUC CUGAUGAG X CGAA ACGCCGG | CCGGCGU U GACGGGG |
| 1493 | UGUAAGU CUGAUGAG X CGAA ACCCCGU | ACGGGGU C ACUUACA |
| 1497 | GUCGUGU CUGAUGAG X CGAA AGUGACC | GGUCACU U ACACGAC |
| 1498 | UGUCGUG CUGAUGAG X CGAA AAGUGAC | GUCACUU A CACGACA |
| 1513 | AGCUUGC CUGAUGAG X CGAA ACCCCCC | GGGGGGU C GCAAGCU |
| 1521 | GUGUGGC CUGAUGAG X CGAA AGCUUGC | GCAAGCU C GCCACAC |
| 1538 | AGGACGU CUGAUGAG X CGAA ACGCUCU | AGAGCGU C ACGUCCU |
| 1543 | GAAGAAG CUGAUGAG X CGAA ACGUGAC | GUCACGU C CUUCUUC |
| 1546 | GGUGAAG CUGAUGAG X CGAA AGGACGU | ACGUCCU U CUUCACC |
| 1547 | GGGUGAA CUGAUGAG X CGAA AAGGACG | CGUCCUU C UUCACCC |
| 1549 | UUGGGUG CUGAUGAG X CGAA AGAAGGA | UCCUUCU U CACCCAA |
| 1550 | CUUGGGU CUGAUGAG X CGAA AAGAAGG | CCUUCUU C ACCCAAG |
| 1574 | UGAGCUG CUGAUGAG X CGAA AUUCUCU | AGAGAAU C CAGCUCA |
| 1580 | UGUUUAU CUGAUGAG X CGAA AGCUGGA | UCCAGCU C AUAAACA |
| 1583 | UGGUGUU CUGAUGAG X CGAA AUGAGCU | AGCUCAU A AACACCA |
| 1607 | UCCUGUU CUGAUGAG X CGAA AUGUGCC | GGCACAU C AACAGGA |
| 1636 | GUUGAGG CUGAUGAG X CGAA AUUCAUU | AAUGAAU C CCUCAAC |
| 1640 | CGGUGUU CUGAUGAG X CGAA AGGGAUU | AAUCCCU C AACACCG |
| 1651 | GGCAAAG CUGAUGAG X CGAA ACCCGGU | ACCGGGU U CUUUGCC |
| 1652 | CGGCAAA CUGAUGAG X CGAA AACCCGG | CCGGGUU C UUUGCCG |
| 1654 | UGCGGCA CUGAUGAG X CGAA AGAACCC | GGGUUCU U UGCCGCA |
| 1655 | GUGCGGC CUGAUGAG X CGAA AAGAACC | GGUUCUU U GCCGCAC |
| 1666 | UGCGUAG CUGAUGAG X CGAA ACAGUGC | GCACUGU U CUACGCA |
| 1667 | GUGCGUA CUGAUGAG X CGAA AACAGUG | CACUGUU C UACGCAC |
| 1669 | GUGUGCG CUGAUGAG X CGAA AGAACAG | CUGUUCU A CGCACAC |
| 1681 | CGAGUUG CUGAUGAG X CGAA ACUUGUG | CACAAGU U CAACUCG |
| 1682 | ACGAGUU CUGAUGAG X CGAA AACUUGU | ACAAGUU C AACUCGU |
| 1687 | UCCGGAC CUGAUGAG X CGAA AGUUGAA | UUCAACU C GUCCGGA |
| 1690 | GCAUCCG CUGAUGAG X CGAA ACGAGUU | AACUCGU C CGGAUGC |
| 1723 | GUCGAUG CUGAUGAG X CGAA AGCUGCA | UGCAGCU C CAUCGAC |
| 1764 | GGCUCGG CUGAUGAG X CGAA AUAGGUG | CACCUAU A CCGAGCC |
| 1773 | AGGUCCC CUGAUGAG X CGAA AGGCUCG | CGAGCCU A GGGACCU |
| 1785 | GGCCUCU CUGAUGAG X CGAA AUCCAGG | CCUGGAU C AGAGGCC |
| 1794 | CAGCAGU CUGAUGAG X CGAA AGGCCUC | GAGGCCU U ACUGCUG |
| 1861 | GAAACAG CUGAUGAG X CGAA ACACUGG | CCAGUGU A CUGUUUC |
| 1866 | GGGGUGA CUGAUGAG X CGAA ACAGUAC | GUACUGU U UCACCCC |
| 1867 | UGGGGUG CUGAUGAG X CGAA AACAGUA | UACUGUU U CACCCCA |
| 1868 | UUGGGGU CUGAUGAG X CGAA AAACAGU | ACUGUUU C ACCCCAA |
| 1955 | UGUUGAG CUGAUGAG X CGAA AGCAGCA | UGCUGCU U CUCAACA |
| 1956 | UUGUUGA CUGAUGAG X CGAA AAGCAGC | GCUGCUU C UCAACAA |
| 1958 | UGUUGUU CUGAUGAG X CGAA AGAAGCA | UGCUUCU C AACAACA |
| 2020 | CUUGGUG CUGAUGAG X CGAA ACCCAGU | ACUGGGU U CACCAAG |
| 2021 | UCUUGGU CUGAUGAG X CGAA AACCCAG | CUGGGUU C ACCAAGA |
| 2094 | CGAAAGC CUGAUGAG X CGAA AUCCGUG | CACGGAU U GCUUUCG |
| 2098 | CUUCCGA CUGAUGAG X CGAA AGCAAUC | GAUUGCU U UCGGAAG |
| 2099 | GCUUCCG CUGAUGAG X CGAA AAGCAAU | AUUGCUU U CGGAAGC |
| 2100 | UGCUUCC CUGAUGAG X CGAA AAAGCAA | UUGCUUU C GGAAGCA |
| 2157 | AUACACC CUGAUGAG X CGAA AGGUGUU | AACACCU A GGUGUAU |
| 2163 | UCAACUA CUGAUGAG X CGAA ACACCUA | UAGGUGU A UAGUUGA |
| 2165 | AGUCAAC CUGAUGAG X CGAA AUACACC | GGUGUAU A GUUGACU |
| 2168 | GGUAGUC CUGAUGAG X CGAA ACUAUAC | GUAUAGU U GACUACC |
| 2173 | GUAUGGG CUGAUGAG X CGAA AGUCAAC | GUUGACU A CCCAUAC |
| 2179 | GAGCCUG CUGAUGAG X CGAA AUGGGUA | UACCCAU A CAGGCUC |
| 2186 | AGUGCCA CUGAUGAG X CGAA AGCCUGU | ACAGGCU C UGGCACU |
| 2194 | GCAGGGG CUGAUGAG X CGAA AGUGCCA | UGGCACU A CCCCUGC |
| 2207 | UAAAGUU CUGAUGAG X CGAA ACAGUGC | GCACUGU C AACUUUA |
| 2212 | GAUGGUA CUGAUGAG X CGAA AGUUGAC | GUCAACU U UACCAUC |
| 2213 | AGAUGGU CUGAUGAG X CGAA AAGUUGA | UCAACUU U ACCAUCU |
| 2214 | AAGAUGG CUGAUGAG X CGAA AAAGUUG | CAACUUU A CCAUCUU |
| 2222 | UAACCUU CUGAUGAG X CGAA AAGAUGG | CCAUCUU U AAGGUUA |
| 2223 | CUAACCU CUGAUGAG X CGAA AAAGAUG | CAUCUUU A AGGUUAG |
| 2228 | ACAUCCU CUGAUGAG X CGAA ACCUUAA | UUAAGGU U AGGAUGU |
| 2229 | UACAUCC CUGAUGAG X CGAA AACCUUA | UAAGGUU A GGAUGUA |
| 2236 | CCCCACA CUGAUGAG X CGAA ACAUCCU | AGGAUGU A UGUGGGG |
| 2283 | UCUCCUC CUGAUGAG X CGAA AGUCCAG | CUGGACU C GAGGAGA |
| 2366 | AACAGGG CUGAUGAG X CGAA AGUGUCU | AGACACU U CCCUGUU |
| 2367 | GAACAGG CUGAUGAG X CGAA AAGUGUC | GACACUU C CCUGUUC |
| 2373 | GUGAAGG CUGAUGAG X CGAA ACAGGGA | UCCCUGU U CCUUCAC |
| 2374 | GGUGAAG CUGAUGAG X CGAA AACAGGG | CCCUGUU C CUUCACC |
| 2377 | GGUGGUG CUGAUGAG X CGAA AGGAACA | UGUUCCU U CACCACC |
| 2378 | GGGUGGU CUGAUGAG X CGAA AAGGAAC | GUUCCUU C ACCACCC |
| 2387 | GAGCCGG CUGAUGAG X CGAA AGGGUGG | CCACCCU A CCGGCUC |
| 2394 | GUGGACA CUGAUGAG X CGAA AGCCGGU | ACCGGCU C UGUCCAC |
| 2398 | ACCAGUG CUGAUGAG X CGAA ACAGAGC | GCUCUGU C CACUGGU |
| 2406 | UGGAUCA CUGAUGAG X CGAA ACCAGUG | CACUGGU U UGAUCCA |
| 2407 | GUGGAUC CUGAUGAG X CGAA AACCAGU | ACUGGUU U GAUCCAC |
| 2411 | GGAGGUG CUGAUGAG X CGAA AUCAAAC | GUUUGAU C CACCUCC |
| 2443 | GUACAGG CUGAUGAG X CGAA ACUGCAC | GUGCAGU A CCUGUAC |
| 2449 | UAUACCG CUGAUGAG X CGAA ACAGGUA | UACCUGU A CGGUAUA |
| 2454 | GACCCUA CUGAUGAG X CGAA ACCGUAC | GUACGGU A UAGGGUC |
| 2456 | CUGACCC CUGAUGAG X CGAA AUACCGU | ACGGUAU A GGGUCAG |
| 2461 | AACCGCU CUGAUGAG X CGAA ACCCUAU | AUAGGGU C AGCGGUU |
| 2468 | AGGAGAC CUGAUGAG X CGAA ACCGCUG | CAGCGGU U GUCUCCU |
| 2471 | CAAAGGA CUGAUGAG X CGAA ACAACCG | CGGUUGU C UCCUUUG |
| 2473 | CACAAAG CUGAUGAG X CGAA AGACAAC | GUUGUCU C CUUUGUG |
| 2476 | GAUCACA CUGAUGAG X CGAA AGGAGAC | GUCUCCU U UGUGAUC |
| 2477 | UGAUCAC CUGAUGAG X CGAA AAGGAGA | UCUCCUU U GUGAUCA |
| 2483 | CCCAUUU CUGAUGAG X CGAA AUCACAA | UUGUGAU C AAAUGGG |
| 2494 | CACGAUA CUGAUGAG X CGAA ACUCCCA | UGGGAGU A UAUCGUG |
| 2496 | AACACGA CUGAUGAG X CGAA AUACUCC | GGAGUAW A UCGUGUU |
| 2498 | GCAACAC CUGAUGAG X CGAA AUAUACU | AGUAUAU C GUGUUGC |
| 2503 | GAAAAGC CUGAUGAG X CGAA ACACGAU | AUCGUGU U GCUUUUC |
| 2507 | GAAGGAA CUGAUGAG X CGAA AGCAACA | UGUUGCU U UUCCUUC |
| 2508 | AGAAGGA CUGAUGAG X CGAA AAGCAAC | GUUGCUU U UCCUUCU |
| 2509 | GAGAAGG CUGAUGAG X CGAA AAAGCAA | UUGCUUU U CCUUCUC |
| 2510 | GGAGAAG CUGAUGAG X CGAA AAAAGCA | UGCUUUU C CUUCUCC |
| 2513 | CCAGGAG CUGAUGAG X CGAA AGGAAAA | UUUUCCU U CUCCUGG |
| 2514 | GCCAGGA CUGAUGAG X CGAA AAGGAAA | UUUCCUU C UCCUGGC |
| 2516 | CCGCCAG CUGAUGAG X CGAA AGAAGGA | UCCUUCU C CUGGCGG |
| 2545 | CAUCCAC CUGAUGAG X CGAA AGCAGGC | GCCUGCU U GUGGAUG |
| 2564 | CCUGGGC CUGAUGAG X CGAA AUCAGCA | UGCUGAU A GCCCAGG |
| 2614 | GGCCAGG CUGAUGAG X CGAA ACGCCGC | GCGGCGU C CCUGGCC |
| 2636 | AGGAGAG CUGAUGAG X CGAA AUGCCAU | AUGGCAU U CUCUCCU |
| 2637 | AAGGAGA CUGAUGAG X CGAA AAUGCCA | UGGCAUU C UCUCCUU |
| 2639 | GGAAGGA CUGAUGAG X CGAA AGAAUGC | GCAUUCU C UCCUUCC |
| 2641 | AAGGAAG CUGAUGAG X CGAA AGAGAAU | AUUCUCU C CUUCCUU |
| 2644 | CACAAGG CUGAUGAG X CGAA AGGAGAG | CUCUCCU U CCUUGUG |
| 2645 | ACACAAG CUGAUGAG X CGAA AAGGAGA | UCUCCUU C CUUGUGU |
| 2648 | AAAACAC CUGAUGAG X CGAA AGGAAGG | CCUUCCU U GUGUUUU |
| 2653 | ACAGAAA CUGAUGAG X CGAA ACACAAG | CUUGUGU U UUUCUGU |
| 2654 | CACAGAA CUGAUGAG X CGAA AACACAA | UUGUGUU U UUCUGUG |
| 2655 | GCACAGA CUGAUGAG X CGAA AAACACA | UGUGUUU U UCUGUGC |
| 2656 | GGCACAG CUGAUGAG X CGAA AAAACAC | GUGUUUU U CUGUGCC |
| 2657 | CGGCACA CUGAUGAG X CGAA AAAAACA | UGUUUUU C UGUGCCG |
| 2732 | GGAGCAG CUGAUGAG X CGAA AGCAGCG | CGCUGCU C CUGCUCC |
| 2749 | UGGUGGU CUGAUGAG X CGAA ACGCCAG | CUGGCGU U ACCACCA |
| 2750 | GUGGUGG CUGAUGAG X CGAA AACGCCA | UGGCGUU A CCACCAC |
| 2791 | UCCACAC CUGAUGAG X CGAA AUGCAGC | GCUGCAU C GUGUGGA |
| 2807 | CUACAAA CUGAUGAG X CGAA ACCACCC | GGGUGGU U UUUGUAG |
| 2808 | CCUACAA CUGAUGAG X CGAA AACCACC | GGUGGUU U UUGUAGG |
| 2809 | ACCUACA CUGAUGAG X CGAA AAACCAC | GUGGUUU U UGUAGGU |
| 2810 | GACCUAC CUGAUGAG X CGAA AAAACCA | UGGUUUU U GUAGGUC |
| 2813 | UUAGACC CUGAUGAG X CGAA ACAAAAA | UUUUUGU A GGUCUAA |
| 2817 | AGUAUUA CUGAUGAG X CGAA ACCUACA | UGUAGGU C UAAUACU |
| 2819 | AGAGUAU CUGAUGAG X CGAA AGACCUA | UAGGUCU A AUACUCU |
| 2822 | UCAAGAG CUGAUGAG X CGAA AUUAGAC | GUCUAAU A CUCUUGA |
| 2825 | AGGUCAA CUGAUGAG X CGAA AGUAUUA | UAAUACU C UUGACCU |
| 2827 | CAAGGUC CUGAUGAG X CGAA AGAGUAU | AUACUCU U GACCUUG |
| 2833 | UGGUGAC CUGAUGAG X CGAA AGGUCAA | UUGACCU U GUCACCA |
| 2836 | GUGUGGU CUGAUGAG X CGAA ACAAGGU | ACCUUGU C ACCACAC |
| 2845 | CACUUUG CUGAUGAG X CGAA AGUGUGG | CCACACU A CAAAGUG |
| 2854 | GGCGAGG CUGAUGAG X CGAA ACACUUU | AAAGUGU U CCUCGCC |
| 2855 | UGGCGAG CUGAUGAG X CGAA AACACUU | AAGUGUU C CUCGCCA |
| 2858 | GCCUGGC CUGAUGAG X CGAA AGGAACA | UGUUCCU C GCCAGGC |
| 2867 | ACCAUAU CUGAUGAG X CGAA AGCCUGG | CCAGGCU C AUAUGGU |
| 2870 | ACCACCA CUGAUGAG X CGAA AUGAGCC | GGCUCAU A UGGUGGU |
| 2889 | CUGGUGA CUGAUGAG X CGAA AAAGUAU | AUACUUU A UCACCAG |
| 2891 | CCCUGGU CUGAUGAG X CGAA AUAAAGU | ACUUUAU C ACCAGGG |
| 2993 | CAAAGAU CUGAUGAG X CGAA AGCUCUG | CAGAGCU A AUCUUUG |
| 2996 | UGUCAAA CUGAUGAG X CGAA AUUAGCU | AGCUAAU C UUUGACA |
| 2998 | AAUGUCA CUGAUGAG X CGAA AGAUUAG | CUAAUCU U UGACAUU |
| 2999 | UAAUGUC CUGAUGAG X CGAA AAGAUUA | UAAUCUU U GACAUUA |
| 3005 | GUUUGGU CUGAUGAG X CGAA AUGUCAA | UUGACAU U ACCAAAC |
| 3006 | AGUUUGG CUGAUGAG X CGAA AAUGUCA | UGACAUU A CCAAACU |
| 3014 | CGAGCAG CUGAUGAG X CGAA AGUUUGG | CCAAACU C CUGCUCG |
| 3020 | GAAUGGC CUGAUGAG X CGAA AGCAGGA | UCCUGCU C GCCAUUC |
| 3026 | GACCGAG CUGAUGAG X CGAA AUGGCGA | UCGCCAU U CUCGGUC |
| 3027 | GGACCGA CUGAUGAG X CGAA AAUGGCG | CGCCAUU C UCGGUCC |
| 3029 | GCGGACC CUGAUGAG X CGAA AGAAUGG | CCAUUCU C GGUCCGC |
| 3033 | AUGAGCG CUGAUGAG X CGAA ACCGAGA | UCUCGGU C CGCUCAU |
| 3038 | GCACCAU CUGAUGAG X CGAA AGCGGAC | GUCCGCU C AUGGUGC |
| 3047 | CAGCCUG CUGAUGAG X CGAA AGCACCA | UGGUGCU C CAGGCUG |
| 3073 | UACAAAG CUGAUGAG X CGAA ACGGCAU | AUGCCGU A CUUUGUA |
| 3076 | GCGUACA CUGAUGAG X CGAA AGUACGG | CCGUACU U UGUACGC |
| 3077 | CGCGUAC CUGAUGAG X CGAA AAGUACG | CGUACUU U GUACGCG |
| 3080 | GAGCGCG CUGAUGAG X CGAA ACAAAGU | ACUUUGU A CGCGCUC |
| 3087 | AGCCCCU CUGAUGAG X CGAA AGCGCGU | ACGCGCU C AGGGGCU |
| 3095 | CACGAAU CUGAUGAG X CGAA AGCCCCU | AGGGGCU U AUUCGUG |
| 3096 | GCACGAA CUGAUGAG X CGAA AAGCCCC | GGGGCUU A UUCGUGC |
| 3098 | AUGCACG CUGAUGAG X CGAA AUAAGCC | GGCUUAU U CGUGCAU |
| 3099 | CAUGCAC CUGAUGAG X CGAA AAUAAGC | GCUUAUU C GUGCAUG |
| 3112 | CCGCACC CUGAUGAG X CGAA ACAUGCA | UGCAUGU U GGUGCGG |
| 3125 | CUCCGGC CUGAUGAG X CGAA ACUUUCC | GGAAAGU A GCCGGAG |
| 3180 | ACGUACG CUGAUGAG X CGAA ACCUGUC | GACAGGU A CGUACGU |
| 3184 | AUAGACG CUGAUGAG X CGAA ACGUACC | GGUACGU A CGUCUAU |
| 3188 | GGUCAUA CUGAUGAG X CGAA ACGUACG | CGUACGU C UAUGACC |
| 3190 | AUGGUCA CUGAUGAG X CGAA AGACGUA | UACGUCU A UGACCAU |
| 3198 | GGGGUAA CUGAUGAG X CGAA AUGGUCA | UGACCAU C UUACCCC |
| 3200 | GCGGGGU CUGAUGAG X CGAA AGAUGGU | ACCAUCU U ACCCCGC |
| 3201 | AGCGGGG CUGAUGAG X CGAA AAGAUGG | CCAUCUU A CCCCGCU |
| 3254 | CGGGCUC CUGAUGAG X CGAA ACUGCCA | UGGCAGU A GAGCCCG |
| 3269 | UGUCAGA CUGAUGAG X CGAA AAGACGA | UCGUCUU C UCUGACA |
| 3271 | CAUGUCA CUGAUGAG X CGAA AGAAGAC | GUCUUCU C UGACAUG |
| 3374 | GUCCCAG CUGAUGAG X CGAA AGUAUCU | AGAUACU U CUGGGAC |
| 3375 | GGUCCCA CUGAUGAG X CGAA AAGUAUC | GAUACUU C UGGGACC |
| 3390 | UCAAUGC CUGAUGAG X CGAA AUCGGCC | GGCCGAU A GCAUUGA |
| 3395 | GCCCUUC CUGAUGAG X CGAA AUGCUAU | AUAGCAU U GAAGGGC |
| 3436 | UUGGGCG CUGAUGAG X CGAA AGGCCGU | ACGGCCU A CGCCCAA |
| 3458 | AACCAAG CUGAUGAG X CGAA AGGCCCC | GGGGCCU A CUUGGUU |
| 3461 | UGCAACC CUGAUGAG X CGAA AGUAGGC | GCCUACU U GGUUGCA |
| 3465 | ACAAUGC CUGAUGAG X CGAA ACCAAGU | ACUUGGU U GCAUUGU |
| 3470 | UAGUAAC CUGAUGAG X CGAA AUGCAAC | GUUGCAU U GUUACUA |
| 3473 | GGCUAGU CUGAUGAG X CGAA ACAAUGC | GCAUUGU U ACUAGCC |
| 3474 | AGGCUAG CUGAUGAG X CGAA AACAAUG | CAUUGUU A CUAGCCU |
| 3477 | GUGAGGC CUGAUGAG X CGAA AGUAACA | UGUUACU A GCCUCAC |
| 3506 | CCCCUUC CUGAUGAG X CGAA ACCUGGU | ACCAGGU C GAAGGGG |
| 3544 | CAGGAAA CUGAUGAG X CGAA AUUGUGU | ACACAAU C UUUCCUG |
| 3546 | GCCAGGA CUGAUGAG X CGAA AGAUUGU | ACAAUCU U UCCUGGC |
| 3547 | CGCCAGG CUGAUGAG X CGAA AAGAUUG | CAAUCUU U CCUGGCG |
| 3548 | UCGCCAG CUGAUGAG X CGAA AAAGAUU | AAUCUUU C CUGGCGA |
| 3563 | CACCAUU CUGAUGAG X CGAA ACGCAGG | CCUGCGU U AAUGGUG |
| 3564 | ACACCAU CUGAUGAG X CGAA AACGCAG | CUGCGUU A AUGGUGU |
| 3584 | CGUGGAA CUGAUGAG X CGAA ACGGUCC | GGACCGU C UUCCACG |
| 3586 | GCCGUGG CUGAUGAG X CGAA AGACGGU | ACCGUCU U CCACGGC |
| 3587 | CGCCGUG CUGAUGAG X CGAA AAGACGG | CCGUCUU C CACGGCG |
| 3632 | UUUGGGU CUGAUGAG X CGAA AUUGGGC | GCCCAAU C ACCCAAA |
| 3643 | AUUAGUG CUGAUGAG X CGAA ACAUUUG | CAAAUGU A CACUAAU |
| 3648 | UCUACAU CUGAUGAG X CGAA AGUGUAC | GUACACU A AUGUAGA |
| 3653 | CUUGGUC CUGAUGAG X CGAA ACAUUAG | CUAAUGU A GACCAAG |
| 3665 | AGCCGAC CUGAUGAG X CGAA AGGUCUU | AAGACCU C GUCGGCU |
| 3668 | GCCAGCC CUGAUGAG X CGAA ACGAGGU | ACCUCGU C GGCUGGC |
| 3720 | UCCGAGC CUGAUGAG X CGAA ACCGCAG | CUGCGGU A GCUCGGA |
| 3758 | CCGGAAU CUGAUGAG X CGAA ACGUCAG | CUGACGU C AUUCCGG |
| 3815 | AAUAGGA CUGAUGAG X CGAA ACGGGUC | GACCCGU C UCCUAUU |
| 3817 | CAAAUAG CUGAUGAG X CGAA AGACGGG | CCCGUCU C CUAUUUG |
| 3820 | CUUCAAA CUGAUGAG X CGAA AGGAGAC | GUCUCCU A UUUGAAG |
| 3822 | CCCUUCA CUGAUGAG X CGAA AUAGGAG | CUCCUAU U UGAAGGG |
| 3823 | GCCCUUC CUGAUGAG X CGAA AAUAGGA | UCCUAUU U GAAGGGC |
| 3832 | ACCCGAA CUGAUGAG X CGAA AGCCCUU | AAGGGCU C UUCGGGU |
| 3834 | CCACCCG CUGAUGAG X CGAA AGAGCCC | GGGCUCU U CGGGUGG |
| 3925 | GGGUAUG CUGAUGAG X CGAA AGUCCAC | GUGGACU U CAUACCC |
| 3926 | CGGGUAU CUGAUGAG X CGAA AAGUCCA | UGGACUU C AUACCCG |
| 3929 | CAACGGG CUGAUGAG X CGAA AUGAAGU | ACUUCAU A CCCGUUG |
| 3935 | UAGACUC CUGAUGAG X CGAA ACGGGUA | UACCCGU U GAGUCUA |
| 3940 | UUCCAUA CUGAUGAG X CGAA ACUCAAC | GUUGAGU C UAUGGAA |
| 3942 | GUUUCCA CUGAUGAG X CGAA AGACUCA | UGAGUCU A UGGAAAC |
| 3951 | CGCAUAG CUGAUGAG X CGAA AGUUUCC | GGAAACU A CUAUGCG |
| 3954 | GACCGCA CUGAUGAG X CGAA AGUAGUU | AACUACU A UGCGGUC |
| 3961 | GACCGGG CUGAUGAG X CGAA ACCGCAU | AUGCGGU C CCCGGUC |
| 3968 | CCGUGAA CUGAUGAG X CGAA ACCGGGG | CCCCGGU C UUCACGG |
| 3970 | GUCCGUG CUGAUGAG X CGAA AGACCGG | CCGGUCU U CACGGAC |
| 3971 | UGUCCGU CUGAUGAG X CGAA AAGACCG | CGGUCUU C ACGGACA |
| 3982 | GGGAGAU CUGAUGAG X CGAA AGUUGUC | GACAACU C AUCUCCC |
| 3985 | CGGGGGA CUGAUGAG X CGAA AUGAGUU | AACUCAU C UCCCCCG |
| 3987 | GCCGGGG CUGAUGAG X CGAA AGAUGAG | CUCAUCU C CCCCGGC |
| 3998 | UCUGCGG CUGAUGAG X CGAA ACGGCCG | CGGCCGU A CCGCAGA |
| 4009 | CACUUGG CUGAUGAG X CGAA AUGUCUG | CAGACAU U CCAAGUG |
| 4010 | CCACUUG CUGAUGAG X CGAA AAUGUCU | AGACAUU C CAAGUGG |
| 4023 | GCGUGUA CUGAUGAG X CGAA AUGGGCC | GGCCCAU C UACACGC |
| 4025 | GAGCGUG CUGAUGAG X CGAA AGAUGGG | CCCAUCU A CACGCUC |
| 4032 | CCAGUGG CUGAUGAG X CGAA AGCGUGU | ACACGCU C CCACUGG |
| 4094 | GGACGAG CUGAUGAG X CGAA ACCUUGU | ACAAGGU A CUCGUCC |
| 4097 | UCAGGAC CUGAUGAG X CGAA AGUACCU | AGGUACU C GUCCUGA |
| 4100 | GGUUCAG CUGAUGAG X CGAA ACGAGUA | UACUCGU C CUGAACC |
| 4111 | GGCAACA CUGAUGAG X CGAA AUGGGUU | AACCCAU C UGUUGCC |
| 4126 | AAAACCC CUGAUGAG X CGAA AGGUGGC | GCCACCU U GGGUUUU |
| 4131 | GCCCCAA CUGAUGAG X CGAA ACCCAAG | CUUGGGU U UUGGGGC |
| 4132 | CGCCCCA CUGAUGAG X CGAA AACCCAA | UUGGGUU U UGGGGCG |
| 4133 | ACGCCCC CUGAUGAG X CGAA AAACCCA | UGGGUUU U GGGGCGU |
| 4141 | AGACAUA CUGAUGAG X CGAA ACGCCCC | GGGGCGU A UAUGUCU |
| 4143 | UUAGACA CUGAUGAG X CGAA AUACGCC | GGCGUAU A UGUCUAA |
| 4147 | UGCCUUA CUGAUGAG X CGAA ACAUAUA | UAUAUGU C UAAGGCA |
| 4149 | UGUGCCU CUGAUGAG X CGAA AGACAUA | UAUGUCU A AGGGACA |
| 4161 | GGGUCGG CUGAUGAG X CGAA ACCAUGU | ACAUGGU A CCGACCC |
| 4196 | CCGUGGU CUGAUGAG X CGAA AUGGUCC | GGACCAU U ACCACGG |
| 4197 | CCCGUGG CUGAUGAG X CGAA AAUGGUC | GACCAUU A CCACGGG |
| 4214 | AGUACGU CUGAUGAG X CGAA AUGGGGG | CCCCCAU C ACGUACU |
| 4219 | GGUGGAG CUGAUGAG X CGAA ACGUGAU | AUCACGU A CUCCACC |
| 4222 | AUAGGUG CUGAUGAG X CGAA AGUACGU | ACGUACU C CACCUAU |
| 4257 | CCCCCAG CUGAUGAG X CGAA ACAUCCA | UGGAUGU U CUGGGGG |
| 4258 | GCCCCCA CUGAUGAG X CGAA AACAUCC | GGAUGUU C UGGGGGC |
| 4270 | GAUAUCA CUGAUGAG X CGAA AGGCGCC | GGCGCCU A UGAUAUC |
| 4275 | AUUAUGA CUGAUGAG X CGAA AUCAUAG | CUAUGAU A UCAUAAU |
| 4277 | AUAUUAU CUGAUGAG X CGAA AUAUCAU | AUGAUAU C AUAAUAU |
| 4300 | GUCAGUU CUGAUGAG X CGAA AGUGGCA | UGCCACU C AACUGAC |
| 4309 | GGUAGUC CUGAUGAG X CGAA AGUCAGU | ACUGACU C GACUACC |
| 4314 | AGGAUGG CUGAUGAG X CGAA AGUCGAG | CUCGACU A CCAUCCU |
| 4319 | UGCCCAG CUGAUGAG X CGAA AUGGUAG | CUACCAU C CUGGGCA |
| 4328 | CUGUGCC CUGAUGAG X CGAA AUGCCCA | UGGGCAU C GGCACAG |
| 4389 | GGAGGCG CUGAUGAG X CGAA AGCGGUG | CACCGCU A CGCCUCC |
| 4395 | GAUCCCG CUGAUGAG X CGAA AGGCGUA | UACGCCU C CGGGAUC |
| 4402 | GGUAACC CUGAUGAG X CGAA AUCCCGG | CCGGGAU C GGUUACC |
| 4406 | GCACGGU CUGAUGAG X CGAA ACCGAUC | GAUCGGU U ACCGUGC |
| 4407 | GGCACGG CUGAUGAG X CGAA AACCGAU | AUCGGUU A CCGUGCC |
| 4427 | CCUCCUC CUGAUGAG X CGAA AUAUUUG | CAAAUAU U GAGGAGG |
| 4440 | UUGGACA CUGAUGAG X CGAA AGCCACC | GGUGGCU C UGUCCAA |
| 4465 | GCCAUAG CUGAUGAG X CGAA AGGGGAU | AUCCCCU U CUAUGGC |
| 4466 | UGCCAUA CUGAUGAG X CGAA AAGGGGA | UCCCCUU C UAUGGCA |
| 4468 | CUUGCCA CUGAUGAG X CGAA AGAAGGG | CCCUUCU A UGGCAAG |
| 4512 | AAAAUGA CUGAUGAG X CGAA AUGCCUU | AAGGCAU C UCAUUUU |
| 4514 | AGAAAAU CUGAUGAG X CGAA AGAUGCC | GGCAUCU C AUUUUCU |
| 4517 | GGCAGAA CUGAUGAG X CGAA AUGAGAU | AUCUCAU U UUCUGCC |
| 4518 | UGGCAGA CUGAUGAG X CGAA AAUGAGA | UCUCAUU U UCUGCCA |
| 4519 | GUGGCAG CUGAUGAG X CGAA AAAUGAG | CUCAUUU U CUGCCAC |
| 4520 | AGUGGCA CUGAUGAG X CGAA AAAAUGA | UCAUUUU C UGCCACU |
| 4550 | UUGCGGC CUGAUGAG X CGAA AGCUCAU | AUGAGCU C GCCGCAA |
| 4564 | GAGGCCU CUGAUGAG X CGAA ACAGCUU | AAGCUGU C AGGCCUC |
| 4571 | UGAUUCC CUGAUGAG X CGAA AGGCCUG | CAGGCCU C GGAAUCA |
| 4602 | ACGUCAA CUGAUGAG X CGAA ACCCCGG | CCGGGGU C UUGACGU |
| 4604 | ACACGUC CUGAUGAG X CGAA AGACCCC | GGGGUCU U GACGUGU |
| 4612 | UAUGACG CUGAUGAG X CGAA ACACGUC | GACGUGU C CGUCAUA |
| 4637 | CGAUAAC CUGAUGAG X CGAA ACAUCUC | GAGAUGU C GUUAUCG |
| 4640 | CCACGAU CUGAUGAG X CGAA ACGACAU | AUGUCGU U AUCGUGG |
| 4641 | GCCACGA CUGAUGAG X CGAA AACGACA | UGUCGUU A UCGUGGC |
| 4643 | UUGCCAC CUGAUGAG X CGAA AUAACGA | UCGUUAU C GUGGCAA |
| 4659 | GUCAUUA CUGAUGAG X CGAA AGCGUCU | AGACGCU C UAAUGAC |
| 4661 | CCGUCAU CUGAUGAG X CGAA AGAGCGU | ACGCUCU A AUGACGG |
| 4684 | CGAGUCA CUGAUGAG X CGAA AGUCACC | GGUGACU U UGACUCG |
| 4685 | CCGAGUC CUGAUGAG X CGAA AAGUCAC | GUGACUU U GACUCGG |
| 4690 | GAUCACC CUGAUGAG X CGAA AGUCAAA | UUUGACU C GGUGAUC |
| 4715 | UCUGGGU CUGAUGAG X CGAA ACACAUG | CAUGUGU C ACCCAGA |
| 4727 | UGAAAUC CUGAUGAG X CGAA ACUGUCU | AGACAGU C GAUUUCA |
| 4731 | AAGCUGA CUGAUGAG X CGAA AUCGACU | AGUCGAU U UCAGCUU |
| 4732 | CAAGCUG CUGAUGAG X CGAA AAUCGAC | GUCGAUU U CAGCUUG |
| 4733 | CCAAGCU CUGAUGAG X CGAA AAAUCGA | UCGAUUU C AGCUUGG |
| 4738 | GGGAUCC CUGAUGAG X CGAA AGCUGAA | UUCAGCU U GGAUCCC |
| 4743 | AAGGUGG CUGAUGAG X CGAA AUCCAAG | CUUGGAU C CCACCUU |
| 4750 | AAUGGUA CUGAUGAG X CGAA AGGUGGG | CCCACCU U UACCAUU |
| 4751 | CAAUGGU CUGAUGAG X CGAA AAGGUGG | CCACCUU U ACCAUUG |
| 4752 | UCAAUGG CUGAUGAG X CGAA AAAGGUG | CACCUUU A CCAUUGA |
| 4757 | UCGUCUC CUGAUGAG X CGAA AUGGUAA | UUACCAU U GAGACGA |
| 4824 | CCUCCCC CUGAUGAG X CGAA ACCCCUG | CAGGGGU A GGGGAGG |
| 4835 | ACCUGUA CUGAUGAG X CGAA AUGCCUC | GAGGCAU C UACAGGU |
| 4837 | AAACCUG CUGAUGAG X CGAA AGAUGCC | GGCAUCU A CAGGUUU |
| 4843 | AGUCACA CUGAUGAG X CGAA ACCUGUA | UACAGGU U UGUGACU |
| 4844 | GAGUCAC CUGAUGAG X CGAA AACCUGU | ACAGGUU U GUGACUC |
| 4851 | UCUCCCG CUGAUGAG X CGAA AGUCACA | UGUGACU C CGGGAGA |
| 4867 | CAUGCCC CUGAUGAG X CGAA AGGGCCG | CGGCCCU C GGGCAUG |
| 4876 | AGAAUCG CUGAUGAG X CGAA ACAUGCC | GGCAUGU U CGAUUCU |
| 4877 | AAGAAUC CUGAUGAG X CGAA AACAUGC | GCAUGUU C GAUUCUU |
| 4881 | ACCGAAG CUGAUGAG X CGAA AUCGAAC | GUUCGAU U CUUCGGU |
| 4882 | GACCGAA CUGAUGAG X CGAA AAUCGAA | UUCGAUU C UUCGGUC |
| 4884 | AGGACCG CUGAUGAG X CGAA AGAAUCG | CGAUUCU U CGGUCCU |
| 4885 | CAGGACC CUGAUGAG X CGAA AAGAAUC | GAUUCUU C GGUCCUG |
| 4889 | CACACAG CUGAUGAG X CGAA ACCGAAG | CUUCGGU C CUGUGUG |
| 4903 | CGCGUCA CUGAUGAG X CGAA AGCACUC | GAGUGCU A UGACGCG |
| 5011 | UUCCCAG CUGAUGAG X CGAA ACUCCAG | CUGGAGU U CUGGGAA |
| 5012 | UUUCCCA CUGAUGAG X CGAA AACUCCA | UGGAGUU C UGGGAAA |
| 5024 | CUGUGAA CUGAUGAG X CGAA ACGCUUU | AAAGCGU C UUCACAG |
| 5026 | GCCUGUG CUGAUGAG X CGAA AGACGCU | AGCGUCU U CACAGGC |
| 5027 | GGCCUGU CUGAUGAG X CGAA AAGACGC | GCGUCUU C ACAGGCC |
| 5036 | UGUGGGU CUGAUGAG X CGAA AGGCCUG | CAGGCCU C ACCCACA |
| 5045 | GGGCAUC CUGAUGAG X CGAA AUGUGGG | CCCACAU A GAUGCCC |
| 5056 | GGACAGG CUGAUGAG X CGAA AGUGGGC | GCCCACU U CCUGUCC |
| 5057 | GGGACAG CUGAUGAG X CGAA AAGUGGG | CCCACUU C CUGUCCC |
| 5062 | GGUUUGG CUGAUGAG X CGAA ACAGGAA | UUCCUGU C CCAAACC |
| 5089 | GUAAGGG CUGAUGAG X CGAA AGUUGUC | GACAACU U CCCUUAC |
| 5090 | GGUAAGG CUGAUGAG X CGAA AAGUUGU | ACAACUU C CCUUACC |
| 5094 | ACCAGGU CUGAUGAG X CGAA AGGGAAG | CUUCCCU U ACCUGGU |
| 5095 | UACCAGG CUGAUGAG X CGAA AAGGGAA | UUCCCUU A CCUGGUA |
| 5139 | GGAGGUG CUGAUGAG X CGAA AGCCUGA | UCAGGCU C CACCUCC |
| 5145 | CACGAUG CUGAUGAG X CGAA AGGUGGA | UCCACCU C CAUCGUG |
| 5149 | AUCCCAC CUGAUGAG X CGAA AUGGAGG | CCUCCAU C GUGGGAU |
| 5157 | CACAUUU CUGAUGAG X CGAA AUCCCAC | GUGGGAU C AAAUGUG |
| 5172 | CGUAUGA CUGAUGAG X CGAA ACACUUC | GAAGUGU C UCAUACG |
| 5174 | GCCGUAU CUGAUGAG X CGAA AGACACU | AGUGUCU C AUACGGC |
| 5177 | UAAGCCG CUGAUGAG X CGAA AUGAGAC | GUCUCAU A CGGCUUA |
| 5183 | UAGGUUU CUGAUGAG X CGAA AGCCGUA | UACGGCU U AAACCUA |
| 5184 | GUAGGUU CUGAUGAG X CGAA AAGCCGU | ACGGCUU A AACCUAC |
| 5190 | UGCAGCG CUGAUGAG X CGAA AGGUUUA | UAAACCU A CGCUGCA |
| 5225 | CGGCUCC CUGAUGAG X CGAA AGCCUAU | AUAGGCU A GGAGCCG |
| 5234 | CAUUUUG CUGAUGAG X CGAA ACGGCUC | GAGCCGU U CAAAAUG |
| 5235 | UCAUUUU CUGAUGAG X CGAA AACGGCU | AGCCGUU C AAAAUGA |
| 5246 | UGAGGGU CUGAUGAG X CGAA AUCUCAU | AUGAGAU C ACCCUCA |
| 5252 | GAUGUGU CUGAUGAG X CGAA AGGGUGA | UCACCCU C ACACAUC |
| 5259 | GUUAUGG CUGAUGAG X CGAA AUGUGUG | CACACAU C CCAUAAC |
| 5264 | AUUUGGU CUGAUGAG X CGAA AUGGGAU | AUCCCAU A ACCAAAU |
| 5272 | CAUGAUG CUGAUGAG X CGAA AUUUGGU | ACCAAAU U CAUCAUG |
| 5273 | CCAUGAU CUGAUGAG X CGAA AAUUUGG | CCAAAUU C AUCAUGG |
| 5276 | AUGCCAU CUGAUGAG X CGAA AUGAAUU | AAUUCAU C AUGGCAU |
| 5290 | GUCGGCC CUGAUGAG X CGAA ACAUGCA | UGCAUGU C GGCCGAC |
| 5349 | GCGGCCA CUGAUGAG X CGAA AGCUGCA | UGCAGCU C UGGCCGC |
| 5384 | CCACAAU CUGAUGAG X CGAA ACCACAC | GUGUGGU C AUUGUGG |
| 5387 | UACCCAC CUGAUGAG X CGAA AUGACCA | UGGUCAU U GUGGGUA |
| 5394 | AUGAUCC CUGAUGAG X CGAA ACCCACA | UGUGGGU A GGAUCAU |
| 5402 | CGGACAA CUGAUGAG X CGAA AUGAUCC | GGAUCAU U UUGUCCG |
| 5403 | CCGGACA CUGAUGAG X CGAA AAUGAUC | GAUCAUU U UGUCCGG |
| 5404 | CCCGGAC CUGAUGAG X CGAA AAAUGAU | AUCAUUU U GUCCGGG |
| 5407 | CCUCCCG CUGAUGAG X CGAA ACAAAAU | AUUUUGU C CGGGAGG |
| 5441 | GGUAGAG CUGAUGAG X CGAA ACUUCCC | GGGAAGU C CUCUACC |
| 5444 | CCCGGUA CUGAUGAG X CGAA AGGACUU | AAGUCCU C UACCGGG |
| 5446 | CUCCCGG CUGAUGAG X CGAA AGAGGAC | GUCCUCU A CCGGGAG |
| 5455 | UUCAUCG CUGAUGAG X CGAA ACUCCCG | CGGGAGU U CGAUGAA |
| 5456 | UUUCAUC CUGAUGAG X CGAA AACUCCC | GGGAGUU C GAUGAAA |
| 5479 | GAGGUGU CUGAUGAG X CGAA AGGCGCA | UGCGCCU C ACACCUC |
| 5486 | UGUAAGG CUGAUGAG X CGAA AGGUGUG | CACACCU C CCUUACA |
| 5490 | UCGAUGU CUGAUGAG X CGAA AGGGAGG | CCUCCCU U ACAUCGA |
| 5491 | UUCGAUG CUGAUGAG X CGAA AAGGGAG | CUCCCUU A CAUCGAA |
| 5495 | CCUGUUC CUGAUGAG X CGAA AUGUAAG | CUUACAU C GAACAGG |
| 5513 | GCUCGGC CUGAUGAG X CGAA AGCUGCA | UGCAGCU C GCCGAGC |
| 5540 | GCAACCC CUGAUGAG X CGAA AGUGCCU | AGGCACU C GGGUUGC |
| 5545 | UUGCAGC CUGAUGAG X CGAA ACCCGAG | CUCGGGU U GCUGCAA |
| 5644 | GCUGAUG CUGAUGAG X CGAA AGUUCCA | UGGAACU U CAUCAGC |
| 5645 | CGCUGAU CUGAUGAG X CGAA AAGUUCC | GGAACUU C AUCAGCG |
| 5648 | UCCCGCU CUGAUGAG X CGAA AUGAAGU | ACUUCAU C AGCGGGA |
| 5657 | AAUACUG CUGAUGAG X CGAA AUCCCGC | GCGGGAU A CAGUAUU |
| 5662 | UGCUAAA CUGAUGAG X CGAA ACUGUAU | AUACAGU A UUUAGCA |
| 5664 | CCUGCUA CUGAUGAG X CGAA AUACUGU | ACAGUAU U UAGCAGG |
| 5665 | GCCUGCU CUGAUGAG X CGAA AAUACUG | CAGUAUU U AGCAGGC |
| 5666 | AGCCUGC CUGAUGAG X CGAA AAAUACU | AGUAUUU A GCAGGCU |
| 5677 | CAGAGUG CUGAUGAG X CGAA AUAAGCC | GGCUUAU C CACUCUG |
| 5682 | CCAGGCA CUGAUGAG X CGAA AGUGGAU | AUCCACU C UGCCUGG |
| 5702 | GUGAUGC CUGAUGAG X CGAA AUCGCGG | CCGCGAU A GCAUCAC |
| 5707 | CAUCAGU CUGAUGAG X CGAA AUGCUAU | AUAGCAU C ACUGAUG |
| 5719 | GGCUGUG CUGAUGAG X CGAA AUGCCAU | AUGGCAU U CACAGCC |
| 5720 | AGGCUGU CUGAUGAG X CGAA AAUGCCA | UGGCAUU C ACAGCCU |
| 5728 | GGUGAUA CUGAUGAG X CGAA AGGCUGU | ACAGCCU C UAUCACC |
| 5730 | CUGGUGA CUGAUGAG X CGAA AGAGGCU | AGCCUCU A UCACCAG |
| 5732 | GACUGGU CUGAUGAG X CGAA AUAGAGG | CCUCUAU C ACCAGUC |
| 5739 | GUGAGCG CUGAUGAG X CGAA ACUGGUG | CACCAGU C CGCUCAC |
| 5744 | GGGUGGU CUGAUGAG X CGAA AGCGGAC | GUCCGCU C ACCACCC |
| 5757 | AGGAGGG CUGAUGAG X CGAA AUUCUGG | CCAGAAU A CCCUCCU |
| 5762 | UGAACAG CUGAUGAG X CGAA AGGGUAU | AUACCCU C CUGUUCA |
| 5774 | CCCCUAA CUGAUGAG X CGAA AUGUUGA | UCAACAU C UUAGGGG |
| 5776 | UCCCCCU CUGAUGAG X CGAA AGAUGUU | AACAUCU U AGGGGGA |
| 5777 | AUCCCCC CUGAUGAG X CGAA AAGAUGU | ACAUCUU A GGGGGAU |
| 5796 | GCGAGUU CUGAUGAG X CGAA AGCAGCC | GGCUGCU C AACUCGC |
| 5808 | GCACUGG CUGAUGAG X CGAA AGGAGCG | CGCUCCU C CCAGUGC |
| 5820 | AAGGCCG CUGAUGAG X CGAA AGCAGCA | UGCUGCU U CGGCCUU |
| 5885 | UGUCCAC CUGAUGAG X CGAA AGCACCU | AGGUGCU U GUGGACA |
| 5894 | CCGCCAG CUGAUGAG X CGAA AUGUCCA | UGGACAU U CUGGCGG |
| 5895 | CCCGCCA CUGAUGAG X CGAA AAUGUCC | GGACAUU C UGGCGGG |
| 5986 | AGGGAGC CUGAUGAG X CGAA AGUUAAC | GUUAACU U GCUCCCU |
| 5999 | GGGAGAG CUGAUGAG X CGAA AUGGCAG | CUGCCAU C CUCUCCC |
| 6002 | CGGGGGA CUGAUGAG X CGAA AGGAUGG | CCAUCCU C UCCCCCG |
| 6101 | CGAACGC CUGAUGAG X CGAA AUCAGCC | GGCUGAU A GCGUUCG |
| 6112 | ACCCCGC CUGAUGAG X CGAA AAGCGAA | UUCGCUU C GCGGGGU |
| 6120 | ACGUGGU CUGAUGAG X CGAA ACCCCGC | GCGGGGU A ACCACGU |
| 6128 | UGGGGGA CUGAUGAG X CGAA ACGUGGU | ACCACGU U UCCCCCA |
| 6129 | GUGGGGG CUGAUGAG X CGAA AACGUGG | CCACGUU U CCCCCAC |
| 6130 | CGUGGGG CUGAUGAG X CGAA AAACGUG | CACGUUU C CCCCACG |
| 6142 | AGGCACG CUGAUGAG X CGAA AGUGCGU | ACGCACU A CGUGCCU |
| 6173 | UCUGAGU CUGAUGAG X CGAA ACACGUG | CACGUGU A ACUCAGA |
| 6177 | AGGAUCU CUGAUGAG X CGAA AGUUACA | UGUAACU C AGAUCCU |
| 6182 | UGGAGAG CUGAUGAG X CGAA AUCUGAG | CUCAGAU C CUCUCCA |
| 6185 | GGCUGGA CUGAUGAG X CGAA AGGAUCU | AGAUCCU C UCCAGCC |
| 6187 | GAGGCUG CUGAUGAG X CGAA AGAGGAU | AUCCUCU C CAGCCUC |
| 6194 | UGAUGGU CUGAUGAG X CGAA AGGCUGG | CCAGCCU C ACCAUCA |
| 6200 | GCUGAGU CUGAUGAG X CGAA AUGGUGA | UCACCAU C ACUCAGC |
| 6204 | AGCAGCU CUGAUGAG X CGAA AGUGAUG | CAUCACU C AGCUGCU |
| 6221 | ACUGGUG CUGAUGAG X CGAA AGCCUCU | AGAGGCU U CACCAGU |
| 6222 | CACUGGU CUGAUGAG X CGAA AAGCCUC | GAGGCUU C ACCAGUG |
| 6233 | CCUCAUU CUGAUGAG X CGAA AUCCACU | AGUGGAU U AAUGAGG |
| 6234 | UCCUCAU CUGAUGAG X CGAA AAUCCAC | GUGGAUU A AUGAGGA |
| 6247 | UGGCGUG CUGAUGAG X CGAA AGCAGUC | GACUGCU C CACGCCA |
| 6259 | CGAGCCG CUGAUGAG X CGAA AGCAUGG | CCAUGCU C CGGCUCG |
| 6265 | UAGCCAC CUGAUGAG X CGAA AGCCGGA | UCCGGCU C GUGGCUA |
| 6272 | CAUCCUU CUGAUGAG X CGAA AGCCACG | CGUGGCU A AAGGAUG |
| 6281 | AGUCCCA CUGAUGAG X CGAA ACAUCCU | AGGAUGU U UGGGACU |
| 6282 | CAGUCCC CUGAUGAG X CGAA AACAUCC | GGAUGUU U GGGACUG |
| 6293 | CCGUGCA CUGAUGAG X CGAA AUCCAGU | ACUGGAU A UGCACGG |
| 6304 | GUCAGUC CUGAUGAG X CGAA ACACCGU | ACGGUGU U GACUGAC |
| 6313 | GGUCUUG CUGAUGAG X CGAA AGUCAGU | ACUGACU U CAAGACC |
| 6314 | AGGUCUU CUGAUGAG X CGAA AAGUCAG | CUGACUU C AAGACCU |
| 6326 | UGGACUG CUGAUGAG X CGAA AGCCAGG | CCUGGCU C CAGUCCA |
| 6331 | GAGCUUG CUGAUGAG X CGAA ACUGGAG | CUCCAGU C CAAGCUC |
| 6338 | UCGGCAG CUGAUGAG X CGAA AGCUUGG | CCAAGCU C CUGCCGA |
| 6349 | UCCCGGC CUGAUGAG X CGAA AUUUCGG | CCGAAAU U GCCGGGA |
| 6359 | AGAAAGG CUGAUGAG X CGAA ACUCCCG | CGGGAGU C CCUUUCU |
| 6363 | GAGAAGA CUGAUGAG X CGAA AGGGACU | AGUCCCU U UCUUCUC |
| 6364 | UGAGAAG CUGAUGAG X CGAA AAGGGAC | GUCCCUU U CUUCUCA |
| 6365 | AUGAGAA CUGAUGAG X CGAA AAAGGGA | UCCCUUU C UUCUCAU |
| 6367 | GCAUGAG CUGAUGAG X CGAA AGAAAGG | CCUUUCU U CUCAUGC |
| 6368 | GGCAUGA CUGAUGAG X CGAA AAGAAAG | CUUUCUU C UCAUGCC |
| 6370 | UUGGCAU CUGAUGAG X CGAA AGAAGAA | UUCUUCU C AUGCCAA |
| 6385 | UCCCUUG CUGAUGAG X CGAA ACCCGCG | CGCGGGU A CAAGGGA |
| 6395 | CCCGCCA CUGAUGAG X CGAA ACUCCCU | AGGGAGU C UGGCGGG |
| 6446 | GUCCGGU CUGAUGAG X CGAA AUUUGUG | CACAAAU U ACCGGAC |
| 6447 | UGUCCGG CUGAUGAG X CGAA AAUUUGU | ACAAAUU A CCGGACA |
| 6458 | CGUUUUU CUGAUGAG X CGAA ACAUGUC | GACAUGU C AAAAACG |
| 6468 | CUCAUGG CUGAUGAG X CGAA ACCGUUU | AAACGGU U CCAUGAG |
| 6469 | CCUCAUG CUGAUGAG X CGAA AACCGUU | AACGGUU C CAUGAGG |
| 6479 | GCCCAAC CUGAUGAG X CGAA AUCCUCA | UGAGGAU C GUUGGGC |
| 6482 | UAGGCCC CUGAUGAG X CGAA ACGAUCC | GGAUCGU U GGGCCUA |
| 6489 | CAGGUUU CUGAUGAG X CGAA AGGCCCA | UGCGCCU A AAACCUG |
| 6520 | GAUGGGG CUGAUGAG X CGAA ACGUUCC | GGAACGU U CCCCAUC |
| 6521 | UGAUGGG CUGAUGAG X CGAA AACGUUC | GAACGUU C CCCAUCA |
| 6527 | ACGCGUU CUGAUGAG X CGAA AUGGGGA | UCCCCAU C AACGCGU |
| 6535 | UGUGGUG CUGAUGAG X CGAA ACGCGUU | AACGCGU A CACCACA |
| 6559 | CGCCGGG CUGAUGAG X CGAA AGGGUGU | ACACCCU C CCCGGCG |
| 6610 | CUCCACG CUGAUGAG X CGAA ACUCUUC | GAAGAGU A CGUGGAG |
| 6620 | CCCGCGU CUGAUGAG X CGAA AUCUCCA | UGGAGAU U ACGCGGG |
| 6621 | ACCCGCG CUGAUGAG X CGAA AAUCUCC | GGAGAUU A CGCGGGU |
| 6654 | GUGGUCA CUGAUGAG X CGAA ACCCGUC | GACGGGU A UGACCAC |
| 6689 | GGGCCGG CUGAUGAG X CGAA ACCUGGC | GCCAGGU C CCGGCCC |
| 6781 | GACCUGG CUGAUGAG X CGAA AUGUGAC | GUCACAU U CCAGGUC |
| 6854 | UGGAAGU CUGAUGAG X CGAA AGCACUG | CAGUGCU C ACUUCCA |
| 6858 | AGCAUGG CUGAUGAG X CGAA AGUGAGC | GCUCACU U CCAUGCU |
| 6859 | GAGCAUG CUGAUGAG X CGAA AAGUGAG | CUCACUU C CAUGCUC |
| 6866 | GGUCGGU CUGAUGAG X CGAA AGCAUGG | CCAUGCU C ACCGACC |
| 6877 | AAUGUGG CUGAUGAG X CGAA AGGGGUC | GACCCCU C CCACAUU |
| 6884 | CUGCUGU CUGAUGAG X CGAA AUGUGGG | CCCACAU U ACAGCAG |
| 6885 | UCUGCUG CUGAUGAG X CGAA AAUGUGG | CCACAUU A CAGCAGA |
| 6900 | CUACGUU CUGAUGAG X CGAA AGCCGUC | GACGGCU A AACGUAG |
| 6945 | CUAGCUG CUGAUGAG X CGAA AGAGCUG | CAGCUCU U CAGCUAG |
| 6946 | GCUAGCU CUGAUGAG X CGAA AAGAGCU | AGCUCUU C AGCUAGC |
| 6951 | AAUUGGC CUGAUGAG X CGAA AGCUGAA | UUCAGCU A GCCAAUU |
| 6969 | UUCAAGG CUGAUGAG X CGAA AGGCGCA | UGCGCCU U CCUUGAA |
| 6970 | CUUCAAG CUGAUGAG X CGAA AAGGCGC | GCGCCUU C CUUGAAG |
| 6973 | UGCCUUC CUGAUGAG X CGAA AGGAAGG | CCUUCCU U GAAGGCA |
| 6990 | UGGUGGG CUGAUGAG X CGAA AGUGCAU | AUGCACU A CCCACCA |
| 7003 | GUCCGGG CUGAUGAG X CGAA AGUCAUG | CAUGACU C CCCGGAC |
| 7019 | CCUCGAU CUGAUGAG X CGAA AGGUCAG | CUGACCU C AUCGAGG |
| 7022 | UGGCCUC CUGAUGAG X CGAA AUGAGGU | ACCUCAU C GAGGCCA |
| 7064 | CACGGGU CUGAUGAG X CGAA AUGUUUC | GAAACAU C ACCCGUG |
| 7078 | AUUCUCU CUGAUGAG X CGAA ACUCCAC | GUGGAGU C AGAGAAU |
| 7086 | ACCACCU CUGAUGAG X CGAA AUUCUCU | AGAGAAU A AGGUGGU |
| 7094 | CCAAAAU CUGAUGAG X CGAA ACCACCU | AGGUGGU A AUUUUGG |
| 7097 | AGUCCAA CUGAUGAG X CGAA AUUACCA | UGGUAAU U UUGGACU |
| 7098 | GAGUCCA CUGAUGAG X CGAA AAUUACC | GGUAAUU U UGGACUC |
| 7099 | AGAGUCC CUGAUGAG X CGAA AAAUUAC | GUAAUUU U GGACUCU |
| 7105 | GUCGAAA CUGAUGAG X CGAA AGUCCAA | UUGGACU C UUUCGAC |
| 7107 | GGGUCGA CUGAUGAG X CGAA AGAGUCC | GGACUCU U UCGACCC |
| 7108 | CGGGUCG CUGAUGAG X CGAA AAGAGUC | GACUCUU U CGACCCG |
| 7109 | GCGGGUC CUGAUGAG X CGAA AAAGAGU | ACUCUUU C GACCCGC |
| 7147 | UGCAACG CUGAUGAG X CGAA AUACUUC | GAAGUAU C CGUUGCA |
| 7151 | CUGCUGC CUGAUGAG X CGAA ACGGAUA | UAUCCGU U GCAGCAG |
| 7163 | UUCGCAG CUGAUGAG X CGAA AUCUCUG | CAGAGAU C CUGCGAA |
| 7174 | CUUCUUG CUGAUGAG X CGAA AUUUUCG | CGAAAAU C CAAGAAG |
| 7183 | GGGGGGG CUGAUGAG X CGAA ACUUCUU | AAGAAGU U CCCCCCC |
| 7184 | CGGGGGG CUGAUGAG X CGAA AACUUCU | AGAAGUU C CCCCCCG |
| 7227 | AACAGUG CUGAUGAG X CGAA AGGGUUG | CAACCCU C CACUGUU |
| 7240 | UUUCCAG CUGAUGAG X CGAA ACUCUAA | UUAGAGU C CUGGAAA |
| 7308 | GGUAUUG CUGAUGAG X CGAA AGGGCCC | GGGCCCU C CAAUACC |
| 7313 | GAGGCGG CUGAUGAG X CGAA AUUGGAG | CUCCAAU A CCGCCUC |
| 7320 | UUCCGUG CUGAUGAG X CGAA AGGCGGU | ACCGCCU C CACGGAA |
| 7340 | UCAGAAC CUGAUGAG X CGAA ACCGUCC | GGACGGU U GUUCUGA |
| 7343 | CUGUCAG CUGAUGAG X CGAA ACAACCG | CGGUUGU U CUGACAG |
| 7344 | UCUGUCA CUGAUGAG X CGAA AACAACC | GGUUGUU C UGACAGA |
| 7363 | GGCAGAA CUGAUGAG X CGAA ACACGGU | ACCGUGU C UUCUGCC |
| 7365 | AAGGCAG CUGAUGAG X CGAA AGACACG | CGUGUCU U CUGCCUU |
| 7366 | CAAGGCA CUGAUGAG X CGAA AAGACAC | GUGUCUU C UGCCUUG |
| 7372 | CUCCGCC CUGAUGAG X CGAA AGGCAGA | UCUGCCU U GGCGGAG |
| 7405 | CGAUCCG CUGAUGAG X CGAA AGCUGCC | GGCAGCU C CGGAUCG |
| 7446 | UGAUCGG CUGAUGAG X CGAA AGGGGCG | CGCCCCU C CCGAUCA |
| 7452 | GAGGUCU CUGAUGAG X CGAA AUCGGGA | UCCCGAU C AGACCUC |
| 7459 | GUCGUCA CUGAUGAG X CGAA AGGUCUG | CAGACCU C UGACGAC |
| 7480 | AACGUCA CUGAUGAG X CGAA AUUCUUU | AAAGAAU C UGACGUU |
| 7487 | ACGACUC CUGAUGAG X CGAA ACGUCAG | CUGACGU U GAGUCGU |
| 7492 | GGAGUAC CUGAUGAG X CGAA ACUCAAC | GUUGAGU C GUACUCC |
| 7495 | GGAGGAG CUGAUGAG X CGAA ACGACUC | GAGUCGU A CUCCUCC |
| 7609 | CCAUGUG CUGAUGAG X CGAA AGGACAU | AUGUCCU A CACAUGG |
| 7631 | AUGGCGU CUGAUGAG X CGAA AUCAGGG | CCCUGAU C ACGCCAU |
| 7675 | GUUGCUC CUGAUGAG X CGAA ACGCGUU | AACGCGU U GAGCAAC |
| 7684 | CAGCAGA CUGAUGAG X CGAA AGUUGCU | AGCAACU C UCUGCUG |
| 7686 | CGCAGCA CUGAUGAG X CGAA AGAGUUG | CAACUCU C UGCUGCG |
| 7695 | UUGUGGU CUGAUGAG X CGAA ACGCAGC | GCUGCGU C ACCACAA |
| 7709 | UGGCAUA CUGAUGAG X CGAA ACCAUGU | ACAUGGU C UAUGCCA |
| 7711 | UGUGGCA CUGAUGAG X CGAA AGACCAU | AUGGUCU A UGCCACA |
| 7754 | CAAAGGU CUGAUGAG X CGAA ACCUUCU | AGAAGGU C ACCUUUG |
| 7759 | UCUGUCA CUGAUGAG X CGAA AGGUGAC | GUCACCU U UGACAGA |
| 7760 | GUCUGUC CUGAUGAG X CGAA AAGGUGA | UCACCUU U GACAGAC |
| 7802 | UCUCCUU CUGAUGAG X CGAA AGCACGU | ACGUGCU C AAGGAGA |
| 7825 | AACUGUG CUGAUGAG X CGAA ACGCCUU | AAGGCGU C CACAGUU |
| 7832 | UAGCCUU CUGAUGAG X CGAA ACUGUGG | CCACAGU U AAGGCUA |
| 7833 | UUAGCCU CUGAUGAG X CGAA AACUGUG | CACAGUU A AGGCUAA |
| 7844 | CGGAUAG CUGAUGAG X CGAA AGUUUAG | CUAAACU U CUAUCCG |
| 7845 | ACGGAUA CUGAUGAG X CGAA AAGUUUA | UAAACUU C UAUCCGU |
| 7884 | UUGGCCG CUGAUGAG X CGAA AUGUGGG | CCCACAU U CGGCCAA |
| 7885 | UUUGGCC CUGAUGAG X CGAA AAUGUGG | CCACAUU C GGCCAAA |
| 7922 | GGUUCCG CUGAUGAG X CGAA ACGUCCU | AGGACGU C CGGAACC |
| 7931 | UGCUGGA CUGAUGAG X CGAA AGGUUCC | GGAACCU A UCCAGCA |
| 7933 | CUUGCUG CUGAUGAG X CGAA AUAGGUU | AACCUAU C CAGCAAG |
| 7946 | UGUGGUU CUGAUGAG X CGAA AUGGCCU | AGGCCAU U AACCACA |
| 7947 | AUGUGGU CUGAUGAG X CGAA AAUGGCC | GGCCAUU A ACCACAU |
| 8000 | UGGUGUC CUGAUGAG X CGAA AUUGGUG | CACCAAU U GACACCA |
| 8012 | UUGCCAU CUGAUGAG X CGAA AUGGUGG | CCACCAU C AUGGCAA |
| 8030 | CGCAGAA CUGAUGAG X CGAA ACUUCAC | GUGAAGU U UUCUGCG |
| 8031 | ACGCAGA CUGAUGAG X CGAA AACUUCA | UGAAGUU U UCUGCGU |
| 8032 | GACGCAG CUGAUGAG X CGAA AAACUUC | GAAGUUU U CUGCGUC |
| 8033 | GGACGCA CUGAUGAG X CGAA AAAACUU | AAGUUUU C UGCGUCC |
| 8039 | CCGGUUG CUGAUGAG X CGAA ACGCAGA | UCUGCGU C CAACCGG |
| 8070 | AUAAGGC CUGAUGAG X CGAA AGCUGGC | GCCAGCU C GCCUUAU |
| 8081 | CUGGGAA CUGAUGAG X CGAA ACGAUAA | UUAUCGU A UUCCCAG |
| 8083 | GUCUGGG CUGAUGAG X CGAA AUACGAU | AUCGUAU U CCCAGAC |
| 8084 | GGUCUGG CUGAUGAG X CGAA AAUACGA | UCGUAUU C CCAGACC |
| 8099 | AUACACG CUGAUGAG X CGAA ACUCCCA | UGGGAGU U CGUGUAU |
| 8100 | CAUACAC CUGAUGAG X CGAA AACUCCC | GGGAGUU C GUGUAUG |
| 8105 | UCUCGCA CUGAUGAG X CGAA ACACGAA | UUCGUGU A UGCGAGA |
| 8121 | UCGUAAA CUGAUGAG X CGAA AGCCAUU | AAUGGCU C UUUACGA |
| 8123 | CGUCGUA CUGAUGAG X CGAA AGAGCCA | UGGCUCU U UACGACG |
| 8124 | ACGUCGU CUGAUGAG X CGAA AAGAGCC | GGCUCUU U ACGACGU |
| 8125 | CACGUCG CUGAUGAG X CGAA AAAGAGC | GCUCUUU A CGACGUG |
| 8135 | GGGUGGA CUGAUGAG X CGAA ACCACGU | ACGUGGU C UCCACCC |
| 8137 | AAGGGUG CUGAUGAG X CGAA AGACCAC | GUGGUCU C CACCCUU |
| 8144 | CCUGAGG CUGAUGAG X CGAA AGGGUGG | CCACCCU U CCUCAGG |
| 8145 | GCCUGAG CUGAUGAG X CGAA AAGGGUG | CACCCUU C CUCAGGC |
| 8148 | ACGGCCU CUGAUGAG X CGAA AGGAAGG | CCUUCCU C AGGCCGU |
| 8164 | GUACGAG CUGAUGAG X CGAA AGCCCAU | AUGGGCU C CUCGUAC |
| 8167 | UCCGUAC CUGAUGAG X CGAA AGGAGCC | GGCUCCU C GUACGGA |
| 8177 | AGUACUG CUGAUGAG X CGAA AAUCCGU | ACGGAUU C CAGUACU |
| 8185 | CCCAGGA CUGAUGAG X CGAA AGUACUG | CAGUACU C UCCUGGG |
| 8241 | AAGCCCA CUGAUGAG X CGAA AGGGCUU | AAGCCCU A UGGGCUU |
| 8248 | AUACGAG CUGAUGAG X CGAA AGCCCAU | AUGGGCU U CUCGUAU |
| 8249 | CAUACGA CUGAUGAG X CGAA AAGCCCA | UGGGCUU C UCGUAUG |
| 8251 | GUCAUAC CUGAUGAG X CGAA AGAAGCC | GGCUUCU C GUAUGAC |
| 8254 | GGUGUCA CUGAUGAG X CGAA ACGAGAA | UUCUCGU A UGACACC |
| 8269 | UGAGUCA CUGAUGAG X CGAA AGGACCG | CGCUGCU U UGACUCA |
| 8270 | UUGAGUC CUGAUGAG X CGAA AAGCAGC | GCUGCUU U GACUCAA |
| 8275 | GACUGUU CUGAUGAG X CGAA AGUCAAA | UUUGACU C AACAGUC |
| 8282 | UCUCAGU CUGAUGAG X CGAA ACUGUUG | CAACAGU C ACUGAGA |
| 8297 | CAACACG CUGAUGAG X CGAA AUGUCGC | GCGACAU C CGUGUUG |
| 8303 | ACUCCUC CUGAUGAG X CGAA ACACGGA | UCCGUGU U GAGGAGU |
| 8311 | GUAGAUU CUGAUGAG X CGAA ACUCCUC | GAGGAGU C AAUCUAC |
| 8315 | AUUGGUA CUGAUGAG X CGAA AUUGACU | AGUCAAU C UACCAAU |
| 8317 | ACAUUGG CUGAUGAG X CGAA AGAUUGA | UCAAUCU A CCAAUGU |
| 8325 | AAGUCAC CUGAUGAG X CGAA ACAUUGG | CCAAUGU U GUGACUU |
| 8332 | GGGGGCC CUGAUGAG X CGAA AGUCACA | UGUGACU U GGCCCCC |
| 8400 | UUUGAAU CUGAUGAG X CGAA AGUCAGG | CCUGACU A AUUCAAA |
| 8403 | CCUUUUG CUGAUGAG X CGAA AUUAGUC | GACUAAU U CAAAAGG |
| 8404 | CCCUUUU CUGAUGAG X CGAA AAUUAGU | ACUAAUU C AAAAGGG |
| 8472 | GUGAGGG CUGAUGAG X CGAA AUUGCCG | CGGCAAU A CCCUCAC |
| 8477 | AGCAUGU CUGAUGAG X CGAA AGGGUAU | AUACCCU C ACAUGCU |
| 8485 | UUUCAAG CUGAUGAG X CGAA AGCAUGU | ACAUGCU A CUUGAAA |
| 8488 | GGCUUUC CUGAUGAG X CGAA AGUAGCA | UGCUACU U GAAAGCC |
| 8565 | UCACAGA CUGAUGAG X CGAA AACGACA | UGUCGUU A UCUGUGA |
| 8567 | UUUCACA CUGAUGAG X CGAA AUAACGA | UCGUUAU C UGUGAAA |
| 8606 | AGACUCG CUGAUGAG X CGAA AGGCUCG | CGAGCCU A CGAGUCU |
| 8612 | CCGUGAA CUGAUGAG X CGAA ACUCGUA | UACGAGU C UUCACGG |
| 8614 | CUCCGUG CUGAUGAG X CGAA AGACUCG | CGAGUCU U CACGGAG |
| 8615 | CCUCCGU CUGAUGAG X CGAA AAGACUC | GAGUCUU C ACGGAGG |
| 8625 | CUAGUCA CUGAUGAG X CGAA AGCCUCC | GGAGGCU A UGACUAG |
| 8631 | GAGUACC CUGAUGAG X CGAA AGUCAUA | UAUGACU A GGUACUC |
| 8635 | GGCAGAG CUGAUGAG X CGAA ACCUAGU | ACUAGGU A CUCUGCC |
| 8677 | CAACUCC CUGAUGAG X CGAA AGUCGUA | UACGACU U GGAGUUG |
| 8683 | UGUUAUC CUGAUGAG X CGAA ACUCCAA | UUGGAGU U GAUAACA |
| 8687 | AUGAUGU CUGAUGAG X CGAA AUCAACU | AGUUGAU A ACAUCAU |
| 8692 | GGAGCAU CUGAUGAG X CGAA AUGUUAU | AUAACAU C AUGCUCC |
| 8710 | CGCGACC CUGAUGAG X CGAA ACACGUU | AACGUGU C GGUCGCG |
| 8714 | CGUGCGC CUGAUGAG X CGAA ACCGACA | UGUCGGU C GCGCACG |
| 8743 | GAGGUAG CUGAUGAG X CGAA ACACUCU | AGAGUGU A CUACCUC |
| 8746 | AGUGAGG CUGAUGAG X CGAA AGUACAC | GUGUACU A CCUCACU |
| 8750 | CACGAGU CUGAUGAG X CGAA AGGUAGU | ACUACCU C ACUCGUG |
| 8754 | GGAUCAC CUGAUGAG X CGAA AGUGAGG | CCUCACU C GUGAUCC |
| 8760 | GUGGUGG CUGAUGAG X CGAA AUCACGA | UCGUGAU C CCACCAC |
| 8799 | GUGUGUC CUGAUGAG X CGAA AGCUGUC | GACAGCU A GACACAC |
| 8808 | UUGACUG CUGAUGAG X CGAA AGUGUGU | ACACACU C CAGUCAA |
| 8813 | AGGAGUU CUGAUGAG X CGAA ACUGGAG | CUCCAGU C AACUCCU |
| 8818 | UAGCCAG CUGAUGAG X CGAA AGUUGAC | GUCAACU C CUGGCUA |
| 8825 | UGUUGCC CUGAUGAG X CGAA AGCCAGG | CCUGGCU A GGCAACA |
| 8834 | ACAUGAU CUGAUGAG X CGAA AUGUUGC | GCAACAU C AUCAUGU |
| 8837 | CAUACAU CUGAUGAG X CGAA AUGAUGU | ACAUCAU C AUGUAUG |
| 8870 | UCAUCAA CUGAUGAG X CGAA AUCAUCC | GGAUGAU U UUGAUGA |
| 8872 | AGUCAUC CUGAUGAG X CGAA AAAUCAU | AUGAUUU U GAUGACU |
| 8884 | GGAGAAG CUGAUGAG X CGAA AGUGAGU | ACUCACU U CUUCUCC |
| 8885 | UGGAGAA CUGAUGAG X CGAA AAGUGAG | CUCACUU C UUCUCCA |
| 8887 | GAUGGAG CUGAUGAG X CGAA AGAAGUG | CACUUCU U CUCCAUC |
| 8888 | GGAUGGA CUGAUGAG X CGAA AAGAAGU | ACUUCUU C UCCAUCC |
| 8890 | AAGGAUG CUGAUGAG X CGAA AGAAGAA | UUCUUCU C CAUCCUU |
| 8894 | CUAGAAG CUGAUGAG X CGAA AUGGAGA | UCUCCAU C CUUCUAG |
| 8897 | GGGCUAG CUGAUGAG X CGAA AGGAUGG | CCAUCCU U CUAGCCC |
| 8898 | UGGGCUA CUGAUGAG X CGAA AAGGAUG | CAUCCUU C UAGCCCA |
| 8900 | CCUGGGC CUGAUGAG X CGAA AGAAGGA | UCCUUCU A GCCCAGG |
| 8915 | CCUUUUC CUGAUGAG X CGAA AGCUGUU | AACAGCU U GAAAAGG |
| 8952 | AUGGAGU CUGAUGAG X CGAA ACAGGCC | GGCCUGU U ACUCCAU |
| 8953 | AAUGGAG CUGAUGAG X CGAA AACAGGC | GCCUGUU A CUCCAUU |
| 8956 | CUCAAUG CUGAUGAG X CGAA AGUAACA | UGUUACU C CAUUGAG |
| 8960 | GUGGCUC CUGAUGAG X CGAA AUGGAGU | ACUCCAU U GAGCCAC |
| 8969 | GUAGGUC CUGAUGAG X CGAA AGUGGCU | AGCCACU U GACCUAC |
| 8975 | UCUGAGG CUGAUGAG X CGAA AGGUCAA | UUGACCU A CCUCAGA |
| 8979 | AUGAUCU CUGAUGAG X CGAA AGGUAGG | CCUACCU C AGAUCAU |
| 8984 | GUUGAAU CUGAUGAG X CGAA AUCUGAG | CUCAGAU C AUUCAAC |
| 8987 | GUCGUUG CUGAUGAG X CGAA AUGAUCU | AGAUCAU U CAACGAC |
| 8988 | AGUCGUU CUGAUGAG X CGAA AAUGAUC | GAUCAUU C AACGACU |
| 8996 | GACCAUG CUGAUGAG X CGAA AGUCGUU | AACGACU C CAUGGUC |
| 9003 | GCGCUAA CUGAUGAG X CGAA ACCAUGG | CCAUGGU C UUAGCGC |
| 9005 | AUGCGCU CUGAUGAG X CGAA AGACCAU | AUGGUCU U AGCGCAU |
| 9006 | AAUGCGC CUGAUGAG X CGAA AAGACCA | UGGUCUU A GCGCAUU |
| 9013 | GAGUGAG CUGAUGAG X CGAA AUGCGCU | AGCGCAU U CUCACUC |
| 9014 | GGAGUGA CUGAUGAG X CGAA AAUGCGC | GCGCAUU C UCACUCC |
| 9016 | AUGGAGU CUGAUGAG X CGAA AGAAUGC | GCAUUCU C ACUCCAU |
| 9020 | AACUAUG CUGAUGAG X CGAA AGUGAGA | UCUCACU C CAUAGUU |
| 9024 | GAGUAAC CUGAUGAG X CGAA AUGGAGU | ACUCCAU A GUUACUC |
| 9027 | GGAGAGU CUGAUGAG X CGAA ACUAUGG | CCAUAGU U ACUCUCC |
| 9028 | UGGAGAG CUGAUGAG X CGAA AACUAUG | CAUAGUU A CUCUCCA |
| 9031 | ACCUGGA CUGAUGAG X CGAA AGUAACU | AGUUACU C UCCAGGU |
| 9033 | UCACCUG CUGAUGAG X CGAA AGAGUAA | UUACUCU C CAGGUGA |
| 9044 | CCCUAUU CUGAUGAG X CGAA AUCUCAC | GUGAGAU C AAUAGGG |
| 9048 | GCCACCC CUGAUGAG X CGAA AUUGAUC | GAUCAAU A GGGUGGC |
| 9057 | AGGCAUG CUGAUGAG X CGAA AGCCACC | GGUGGCU U CAUGCCU |
| 9058 | GAGGCAU CUGAUGAG X CGAA AAGCCAC | GUGGCUU C AUGCCUC |
| 9105 | CUGGCCC CUGAUGAG X CGAA AUGUCUC | GAGACAU C GGGCCAG |
| 9169 | GAAGAGG CUGAUGAG X CGAA ACUUGCC | GGCAAGU A CCUCUUC |
| 9173 | AGUUGAA CUGAUGAG X CGAA AGGUACU | AGUACCU C UUCAACU |
| 9175 | CCAGUUG CUGAUGAG X CGAA AGAGGUA | UACCUCU U CAACUGG |
| 9176 | CCCAGUU CUGAUGAG X CGAA AAGAGGU | ACCUCUU C AACUGGG |
| 9188 | UGGUCCU CUGAUGAG X CGAA ACUGCCC | GGGCAGU A AGGACCA |
| 9200 | UGAGUUU CUGAUGAG X CGAA AGCUUGG | CCAAGCU C AAACUCA |
| 9206 | UUGGAGU CUGAUGAG X CGAA AGUUUGA | UCAAACU C ACUCCAA |
| 9210 | GGGAUUG CUGAUGAG X CGAA AGUGAGU | ACUCACU C CAAUCCC |
| 9215 | CGGCCGG CUGAUGAG X CGAA AUUGGAG | CUCCAAU C CCGGCCG |
| 9261 | CCGCUGU CUGAUGAG X CGAA ACCACCA | UGCUGGU U ACAGCGG |
| 9262 | CCCGCUG CUGAUGAG X CGAA AACCAGC | GCUGGUU A CAGCGGG |
| 9294 | CGGGCAC CUGAUGAG X CGAA AGACAGG | CCUGUCU C GUGCCCG |
| 9313 | CCACAUA CUGAUGAG X CGAA ACCAGCG | CGCUGGU U UAUGUGG |
| 9314 | ACCACAU CUGAUGAG X CGAA AACCAGC | GCUGGUU U AUGUGGU |
| 9315 | CACCACA CUGAUGAG X CGAA AAACCAG | CUGGUUU A UGUGGUG |
| 9409 | AAAAGGG CUGAUGAG X CGAA AUGGCCU | AGGCCAU C CCCUUUU |
| 9414 | AAAAAAA CUGAUGAG X CGAA AGGGGAU | AUCCCCU U UUUUUUU |

Where “X” represents stem II region of a HH ribozyme (Hertel et al., 1992 Nucleic Acids Res. 20: 3252). The length of stem II may be ≧2 base-pairs.

TABLE VII
HCV Hairpin (HP) Ribozyme and Target Sequence

| | | |
|---|---|---|
| Pos. | Ribozyme Sequence | Substrate |

| | | |
|---|---|---|
| 10 | CCCCCA AGAA GGGG ACCAGAGAAACA X GUACAUUACCUGGUA | CCCC CGAU UGGGGG |
| 59 | CGUGAA AGAA GUAG ACCAGAGAAACA X GUACAUUACCUGGUA | CUAC UGUC UUCACG |
| 109 | CCUGGA AGAA GCAC ACCAGAGAAACA X GUACAUUACCUGGUA | GUOC AGCC UCCAGG |
| 209 | GCAUUG AGAA GGUU ACCAGAGAAACA X GUACAUUACCUGGUA | AACC CGCU CAAUGC |
| 290 | CUAUCA AGAA GUAC ACCAGAGAAACA X GUACAUUACCUGGUA | GUAC UGCC UGAUAG |
| 390 | GUGGGC AGAA GUAG ACCAGAGAAACA X GUACAUUACCUGGUA | CUAC CGCC GCCCAC |
| 393 | CCUGUG AGAA GCGG ACCAGAGAAACA X GUACAUUACCUGGUA | CCGC CGCC CACAGG |
| 427 | CCAACG AGAA GACC ACCAGAGAAACA X GUACAUUACCUGGUA | OGUC AGAU CGUUGG |
| 505 | GGUUGC AGAA GUUC ACCAGAGAAACA X GUACAUUACCUGGUA | GAAC GGUC GCAACC |
| 549 | CCUCGG AGAA GCGA ACCAGAGAAACA X GUACAUUACCUGGUA | UCGC CGAC CCGAGG |
| 574 | UACCCA AGAA GAGC ACCAGAGAAACA X GUACAUUACCUGGUA | GCUC AGCC UGGGUA |
| 645 | GCCGGG AGAA GCGG ACCAGAGAAACA X GUACAUUACCUGGUA | CCGC GGCU CCCGGC |
| 652 | CAACUA AGAA GOGA ACCAGAGAAACA X GUACAUUACCUGGUA | UCCC GGCC UAGUUG |
| 671 | CCGGGG AGAA GUGG ACCAGAGAAACA X GUACAUUACCUGGUA | CCAC GGAC CCCCGG |
| 726 | CGGCGA AGAA GCAC ACCAGAGAAACA X GUACAUUACCUGGUA | GUGC GGCU UCGCCG |
| 734 | CAUGAG AGAA GCGA ACCAGAGAAACA X GUACAUUACCUGGUA | UCGC CGAC CUCAUG |
| 754 | CCGACG AGAA GAAU ACCAGAGAAACA X GUACAUUACCUGGUA | AUUC CGCU CGUCGG |
| 852 | AAGAGC AGAA GGGC ACCAGAGAAACA X GUACAUUACCUGGUA | GCCC GGUU GCUCUU |
| 883 | CAGGAC AGAA GGGC ACCAGAGAAACA X GUACAUUACCUGGUA | GCCC UGCU GUCCUG |
| 886 | AAACAG AGAA GCAG ACCAGAGAAACA X GUACAUUACCUGGUA | CUGC UGUC CUGUUU |
| 891 | UCCUCA AGAA GGAC ACCAGAGAAACA X GUACAUUACCUGGUA | GUCC UGUU UGACCA |
| 905 | AGGGGA AGAA GGGA ACCAGAGAAACA X GUACAUUACCUGGUA | UCCC AGCU UCCGCU |
| 911 | CUGAUA AGAA GAAG ACCAGAGAAACA X GUACAUUACCUGGUA | CUUC CGCU UAUCAG |
| 960 | AGUUGG AGAA GUCA ACCAGAGAAACA X GUACAUUACCUGGUA | UGAC UGCU CCAACU |
| 1050 | CCCAAC AGAA GGAG ACCAGAGAAACA X GUACAUUACCUGGUA | CUCC CGUW GUUGGG |
| 1145 | GAAAGC AGAA GCCC ACCAGAGAAACA X GUACAUUACCUGGUA | GGGC GGCC GCUUUC |
| 1148 | ACAGAA AGAA GCCG ACCAGAGAAACA X GUACAUUACCUGGUA | CGGC CGCU UUCUGU |
| 1155 | UGGCGG AGAA GAAA ACCAGAGAAACA X GUACAUUACCUGGUA | UUUC UGUU CCGCCA |
| 1185 | AAACGG AGAA GCAG ACCAGAGAAACA X GUACAUUACCUGGUA | CUGC GGAU CCGUUU |
| 1190 | GAGGAA AGAA GAUC ACCAGAGAAACA X GUACAUUACCUGGUA | GAUC CGUU UUCCUC |
| 1207 | GUGAAC AGAA CGCA ACCAGAGAAACA X GUACAUUACCUGGUA | UCCC AGUU GUUCAC |
| 1331 | CACUAG AGAA GUUG ACCAGAGAAACA X GUACAUUACCUGGUA | CAAC AGCC CUAGUG |
| 1357 | UGUGGG AGAA GGAG ACCAGAGAAACA X GUACAUUACCUGGUA | CUCC GGAU CCCACA |
| 1370 | AUCCAC AGAA GCUU ACCAGAGAAACA X GUACAUUACCUGGUA | AAGC UGUC GUGGAU |
| 1562 | UCUCUG AGAA GGCC ACCAGAGAAACA X GUACAUUACCUGGUA | 00CC 00CC CAGAGA |
| 1576 | UUUAUG AGAA GGAU ACCAGAGAAACA X GUACAUUACCUGGUA | AUCC AGCU CAUAAA |
| 1596 | UGUGCC AGAA GCCA ACCAGAGAAACA X GUACAUUACCUGGUA | UGGC AGCU GGCACA |
| 1616 | GUUCAG AGAA GUCC ACCAGAGAAACA X GUACAUUACCUGGUA | GGAC UGCC CUGAAC |
| 1663 | GCCUAG AGAA GUGC ACCAGAGAAACA X GUACAUUACCUGGUA | GCAC UGUU CUACGC |
| 1692 | CUGGGC AGAA GGAC ACCAGAGAAACA X GUACAUUACCUGGUA | GUCC GGAU GCCCAG |
| 1713 | ACCUGC AGAA GGCC ACCAGAGAAACA X GUACAUUACCUGGUA | GGCC AGCU GCAGCU |
| 1719 | CGAUGG AGAA GCAG ACCAGAGAAACA X GUACAUUACCUGGUA | CUGC AGCU CCAUCG |
| 1797 | AAUGCC AGAA GUAA ACCAGAGAAACA X GUACAUUACCUGGUA | UUAC UGCU GGCAUU |
| 1863 | GGGUGA AGAA GUAC ACCAGAGAAACA X GUACAUUACCUGGUA | GUAC UGUU UCACCC |
| 1880 | CACUAC AGAA GGCC ACCAGAGAAACA X GUACAUUACCUGGUA | GGCC UGUU GUAGUG |
| 1898 | GGACCG AGAA GUCG ACCAGAGAAACA X GUACAUUACCUGGUA | CGAC CGAU CGGUCC |
| 1903 | GCACCG AGAA GAUC ACCAGAGAAACA X GUACAUUACCUGGUA | GAUC GGUC CGGUGC |
| 1943 | CAGCAC AGAA GUCU ACCAGAGAAACA X GUACAUUACCUGGUA | AGAC AGAU GUGCUG |
| 1951 | UUGAGA AGAA GCAC ACCAGAGAAACA X GUACAUUACCUGGUA | GUGC UGCU UCUCAA |
| 1969 | UGUGGC AGAA GCGU ACCAGAGAAACA X GUACAUUACCUGGUA | ACGC GGCC GCCACA |
| 2082 | CCGUGG AGAA GGUC ACCAGAGAAACA X GUACAUUACCUGGUA | GACC UGGC CCACGG |
| 2090 | AAAGCA AGAA GUGG ACCAGAGAAACA X GUACAUUACCUGGUA | CCAC GGAU UGCUUU |
| 2316 | GCUCCG AGAA GUCG ACCAGAGAAACA X GUACAUUACCUGGUA | GGAC AGAU CGGAGC |
| 2328 | GCAGCG AGAA GAGG ACCAGAGAAACA X GUACAUUACCUGGUA | GCUC AGGC CGCUGC |
| 2332 | AGCAGC AGAA GGCU ACCAGAGAAACA X GUACAUUACCUGGUA | AGCC CGCU GCUGCU |
| 2335 | GACAGG AGAA GCGG ACCAGAGAAACA X GUACAUUACCUGGUA | CGGC UGGU GGUGUC |
| 2338 | GUGGAC AGAA GCAG ACCAGAGAAACA X GUACAUUACCUGGUA | CUGC UGCU GUCCAC |
| 2341 | GUCGUG AGAA GCAG ACCAGAGAAACA X GUACAUUACCUGGUA | CUGC UGUC CACGAC |
| 2370 | UGAAGG AGAA GGGA ACCAGAGAAACA X GUACAUUACCUGGUA | UCCG UGUU CCUUCA |
| 2390 | GGACAG AGAA GGUA ACCAGAGAAACA X GUACAUUACCUGGUA | UACC GGCU CUGUGC |
| 2395 | CCAGUG AGAA GAGC ACCAGAGAAACA X GUACAUUACCUGGUA | GCUC UGUC CACUGG |
| 2465 | GGAGAC AGAA GCUG ACCAGAGAAACA X GUACAUUACCUGGUA | CAGC GGUU GUCUCC |
| 2522 | GCGCGC AGAA GCCA ACCAGAGAAACA X GUACAUUACCUGGUA | UGGC GGAC GCGCGC |
| 2541 | UCCACA AGAA GGCA ACCAGAGAAACA X GUACAUUACCUGGUA | UGGC UGCU UGUGGA |
| 2557 | GCUAUC AGAA GCAU ACCAGAGAAACA X GUACAUUACCUGGUA | AUGG UGCU GAUAGC |
| 2579 | CUCUAG AGAA GCCU ACCAGAGAAACA X GUACAUUACCUGGUA | AGGG CGCC CUAGAG |
| 2627 | AAUGCC AGAA GCUC ACCAGAGAAACA X GUACAUUACCUGGUA | GAGC GGAU GGCAUU |
| 2663 | GUACCA AGAA GCAC ACCAGAGAAACA X GUACAUUACCUGGUA | GUGC CGCC UGGUAC |
| 2725 | AGGAGC AGAA GGCA ACCAGAGAAACA X GUACAUUACCUGGUA | UGGC CGCU GCUCCU |
| 2728 | AGCAGG AGAA GCGG ACCAGAGAAACA X GUACAUUACCUGGUA | CCGC UGCU CCUGCU |
| 2734 | AGCAGG AGAA GGAG ACCAGAGAAACA X GUACAUUACCUGGUA | CUCC UGCU CCUGCU |
| 2740 | AACGCC AGAA GGAG ACCAGAGAAACA X GUACAUUACCUGGUA | CUCC UGCU GGCGUU |
| 2978 | UGGGUG AGAA GCAC ACCAGAGAAACA X GUACAUUACCUGGUA | GUGC GGCC CACCCA |
| 3016 | AUGGCG AGAA GGAG ACCAGAGAAACA X GUACAUUACCUGGUA | CUCC UGCU CGGCAU |
| 3030 | UGAGCG AGAA GAGA ACCAGAGAAACA X GUACAUUACCUGGUA | UCUC GGUC CGCUCA |
| 3034 | ACCAUG AGAA GACC ACCAGAGAAACA X GUACAUUACCUGGUA | GGUC CGCU CAUGGU |
| 3260 | GAAGAC AGAA GGCU ACCAGAGAAACA X GUACAUUACCUGGUA | AGCC CGUC GUCUUC |
| 3340 | GAGACG AGAA GUCG ACCAGAGAAACA X GUACAUUACCUGGUA | GGAC UGGC CGUCUC |
| 3344 | GGCGGA AGAA GGCA ACCAGAGAAACA X GUACAUUACCUGGUA | UGGC CGUC UCCGGC |
| 3350 | CCUUCG AGAA GAGA ACCAGAGAAACA X GUACAUUACCUGGUA | UCUC CGCC CGAAGG |
| 3383 | GCUAUC AGAA GGUC ACCAGAGAAACA X GUACAUUACCUGGUA | GACC GGCC GAUAGC |
| 3431 | GGCGUA AGAA GUGA ACCAGAGAAACA X GUACAUUACCUGGUA | UCAC GGCC UACGCC |
| 3581 | GUGGAA AGAA GUGC ACCAGAGAAACA X GUACAUUACCUGGUA | GGAC CGUC UUCCAC |
| 3597 | UCUUUG AGAA GGCG ACCAGAGAAACA X GUACAUUACCUGGUA | CGGC GGCU CAAAGA |
| 3615 | CUUUUG AGAA GGCU ACCAGAGAAACA X GUACAUUACCUGGUA | AGGC GGCC CAAAAG |
| 3669 | CAUGCC AGAA GACG ACCAGAGAAACA X GUACAUUACCUGGUA | CGUC GGCU GGCAUG |
| 3725 | AUAGAG AGAA GAGC ACCAGAGAAACA X GUACAUUACCUCGUA | CCUC GGAC CUCUAU |
| 3752 | AAUGAC AGAA GCAU ACCAGAGAAACA X GUACAUUACCUGGUA | AUGC UGAC GUCAUU |
| 3771 | CACCGC AGAA GGGG ACCAGAGAAACA X GUACAUUACCUGGUA | GCGC CGAC GCGGUG |
| 3783 | UCCCCC AGAA GUCA ACCAGAGAAACA X GUACAUUACCUGGUA | UGAC GGUC GGGGGA |
| 3799 | CUGGGG AGAA GUAG ACCAGAGAAACA X GUACAUUACCUGGUA | CUAC UGUC CCCCAG |
| 3807 | AGACGG AGAA GGGG ACCAGAGAAACA X GUACAUUACCUGGUA | CCCC AGAC CCGUCU |
| 3812 | AUAGGA AGAA GGUC ACCAGAGAAACA X GUACAUUACCUGGUA | GACC CGUC UCCUAU |
| 3847 | GGGCAG AGAA GUGG ACCAGAGAAACA X GUACAUUACCUGGUA | CCAC UGCU CUGCCC |
| 3852 | CCGAAG AGAA GAGC ACCAGAGAAACA X GUACAUUACCUGGUA | GCUC UGCC CUUCGG |
| 3887 | GCACAC AGAA GCCC ACCAGAGAAACA X GUACAUUACCUGGUA | GGGC UGCU GUGUGC |
| 3932 | AGACUC AGAA GGUA ACCAGAGAAACA X GUACAUUACCUGGUA | UACC CGUU GAGUCU |
| 3958 | ACCGGG AGAA GCAU ACCAGAGAAACA X GUACAUUACCUGGUA | AUGC GGUC CCCGGU |
| 3965 | CGUGAA AGAA GGGG ACCAGAGAAACA X GUACAUUACCUGGUA | CCCC GGUC UUCACG |
| 3992 | CGGUAC AGAA GGGG ACCAGAGAAACA X GUACAUUACCUGGUA | CCCC GGCC GUACCG |
| 4064 | GUACGC AGAA GGCA ACCAGAGAAACA X GUACAUUACCUGGUA | UGCC GGCU GCGUAC |
| 4076 | CCCUUG AGAA GCGU ACCAGAGAAACA X GUACAUUACCUGGUA | ACGC AGCC CAAGGG |
| 4112 | GGCGGC AGAA GAUG ACCAGAGAAACA X GUACAUUACCUGGUA | CAUC UGUU GCCGCC |
| 4163 | GUUGGG AGAA GUAC ACCAGAGAAACA X GUACAUUACCUGGUA | GUAC CGAC CCCAAC |
| 4244 | UCCACC AGAA GCAA ACCAGAGAAACA X GUACAUUACCUGGUA | UUGC CGAC GGUGGA |
| 4304 | AGUCGA AGAA GUUG ACCAGAGAAACA X GUACAUUACCUGGUA | CAAC UGAC UCGACU |
| 4334 | GUCCAG AGAA GUGC ACCAGAGAAACA X GUACAUUACCUGGUA | GCAC AGUC CUGGAC |
| 4355 | CGCUCC AGAA GUCU ACCAGAGAAACA X GUACAUUACCUGGUA | AGAC GGCU GGAGCG |
| 4366 | ACGACG AGAA GCGC ACCAGAGAAACA X GUACAUUACCUGGUA | GCGC GGCU CGUCGU |
| 4441 | GUGUUG AGAA GAGC ACCAGAGAAACA X GUACAUUACCUGGUA | GCUC UGUC CAACAC |
| 4621 | CCGCUA AGAA GUAU ACCAGAGAAACA X GUACAUUACCUGGUA | AUAC CGAC UAGCGG |
| 4652 | UAGAGC AGAA GUUG ACCAGAGAAACA X GUACAUUACCUGGUA | CAAC AGAC GCUCUA |
| 4724 | GAAAUC AGAA GUCU ACCAGAGAAACA X GUACAUUACCUGGUA | AGAC AGUC GAUUUC |
| 4734 | GAUCCA AGAA GAAA ACCAGAGAAACA X GUACAUUACCUGGUA | UUUC AGCU UGGAUC |
| 4861 | CCCGAG AGAA GUUC ACCAGAGAAACA X GUACAUUACCUGGUA | GAAC GGCC CUCGGG |
| 4886 | ACACAG AGAA GAAG ACCAGAGAAACA X GUACAUUACCUGGUA | CUUC GGUC CUGUGU |
| 4937 | AGUCUC AGAA GGCG ACCAGAGAAACA X GUACAUUACCUGGUA | CGGC CGCU GAGACU |
| 4988 | CUGGCA AGAA GGCA ACCAGAGAAACA X GUACAUUACCUGGUA | UGCC CGUC UGCCAG |
| 5059 | GUUUGG AGAA GGAA ACCAGAGAAACA X GUACAUUACCUGGUA | UUCC UGUC CCAAAC |
| 5179 | GGUUUA AGAA GUAU ACCAGAGAAACA X GUACAUUACCUGGUA | AUAC GGCU UAAACC |
| 5212 | CUAUAC AGAA GGGG ACCAGAGAAACA X GUACAUUACCUGGUA | CCCC UGCU GUAUAG |
| 5231 | AUUUUG AGAA GCUC ACCAGAGAAACA X GUACAUUACCUGGUA | GAGG CGUU CAAAAU |
| 5291 | CAGGUC AGAA GACA ACCAGAGAAACA X GUACAUUACCUGGUA | UGUC GGCC GACCUG |
| 5294 | CUCCAG AGAA GCCG ACCAGAGAAACA X GUACAUUACCUGGUA | CGGC CGAC CUGGAG |
| 5345 | GGCCAG AGAA GCAA ACCAGAGAAACA X GUACAUUACCUGGUA | UUGC AGCU CUGGCC |
| 5417 | AACAAC AGAA GGCC ACCAGAGAAACA X GUACAUUACCUGGUA | GGCC GGCU GUUGUU |
| 5420 | GGGAAC AGAA GCCG ACCAGAGAAACA X GUACAUUACCUGGUA | CGGC UGUU GUUCCC |
| 5509 | UCGGCG AGAA GCAU ACCAGAGAAACA X GUACAUUACCUGGUA | AUGC AGCU CGGCGA |
| 5521 | UGCUUG AGAA GCUC ACCAGAGAAACA X GUACAUUACCUGGUA | GAGC AGUU CAAGCA |
| 5576 | GGGAGC AGAA GGCU ACCAGAGAAACA X GUACAUUACCUGGUA | AGGC CGCU GCUCCC |
| 5579 | CACGGG AGAA GGGG ACCAGAGAAACA X GUACAUUACCUGGUA | CCGG UGCU CCCGUG |
| 5683 | UUCCCA AGAA GAGU ACCAGAGAAACA X GUACAUUACCUGGUA | ACUC UGCC UGGGAA |
| 5710 | AAUGCC AGAA GUGA ACCAGAGAAACA X GUACAUUACCUGGUA | UCAC UGAU GGCAUU |
| 5723 | GAUAGA AGAA GUGA ACCAGAGAAACA X GUACAUUACCUCGUA | UCAC AGCC UCUAUC |
| 5736 | UGAGCG AGAA GGUG ACCAGAGAAACA X GUACAUUACCUGGUA | CACC AGUC CGCUCA |
| 5740 | GUGGUG AGAA GACU ACCAGAGAAACA X GUACAUUACCUGGUA | AGUC CGCU CACCAC |
| 5764 | AUGUUG AGAA GGAG ACCAGAGAAACA X GUACAUUACCUGGUA | CUCC UGUU CAACAU |
| 5792 | GAGUUG AGAA GCCA ACCAGAGAAACA X GUACAUUACCUGGUA | UGGC UGCU CAACUC |
| 5816 | GGCCGA AGAA GCAC ACCAGAGAAACA X GUACAUUACCUGGUA | GUGC UGCU UCGGCC |
| 5822 | CACGAA AGAA GAAG ACCAGAGAAACA X GUACAUUACCUGGUA | CUUC GGCC UUCGUG |
| 5966 | GUCCUC AGAA GAGG ACCAGAGAAACA X GUACAUUACCUGGUA | CCUC CGCC GAGGAC |
| 6094 | GCUAUC AGAA GGUU ACCAGAGAAACA X GUACAUUACCUGGUA | AACC GGUU GAUAGC |
| 6178 | GAGAGG AGAA GAGU ACCAGAGAAACA X GUACAUUACCUGGUA | ACUC AGAU CCUCUC |
| 6189 | UGGUGA AGAA GGAG ACCAGAGAAACA X GUACAUUACCUGGUA | CUCC AGCC UCACCA |
| 6205 | UUCAGC AGAA GAGU ACCAGAGAAACA X GUACAUUACCUGGUA | ACUC AGCU GCUGAA |
| 6208 | CUCUUC AGAA GCUG ACCAGAGAAACA X GUACAUUACCUGGUA | CAGC UGCU GAAGAG |
| 6243 | GGGUGG AGAA GUCC ACCAGAGAAACA X GUACAUUACCUGGUA | GGAC UGCU CGACGC |
| 6261 | GCCACG AGAA GGAG ACCAGAGAAACA X GUACAUUACCUGGUA | CUCC GGCU CGUGGC |
| 6308 | CUUGAA AGAA GUCA ACCAGAGAAACA X GUACAUUACCUGGUA | UGAC UGAC UUCAAG |
| 6328 | AGCUUG AGAA GGAG ACCAGAGAAACA X GUACAUUACCUGGUA | CUCC AGUC CAAGCU |
| 6340 | AAUUUC AGAA GGAG ACCAGAGAAACA X GUACAUUACCUGGUA | CUCC UGCC GAAAUU |
| 6426 | CACAUG AGAA GGUG ACCAGAGAAACA X GUACAUUACCUGGUA | CACC UGCC CAUGUG |
| 6465 | UCAUGG AGAA GUUU ACCAGAGAAACA X GUACAUUACCUGGUA | AAAC GGUU CCAUGA |
| 6599 | CUCUUC AGAA GCCA ACCAGAGAAACA X GUACAUUACCUGGUA | UGGC UGCU GAAGAG |
| 6692 | UUCGGG AGAA GGGA ACCAGAGAAACA X GUACAUUACCUGGUA | UCCC GGCC CCCGAA |
| 6727 | CUGUGC AGAA GCAC ACCAGAGAAACA X GUACAUUACCUGGUA | GUGC GGUU GCACAG |
| 6753 | GGAGAG AGAA GCAC ACCAGAGAAACA X GUACAUUACCUGGUA | GUGC AGAC CUCUCC |
| 6817 | CAUGGG AGAA GUGA ACCAGAGAAACA X GUACAUUACCUGGUA | UCAC AGCU CCCAUG |
| 6839 | UGCCAC AGAA GGUU ACCAGAGAAACA X GUACAUUACCUGGUA | AACC GGAU GUGGCA |
| 6869 | GGAGGG AGAA GUGA ACCAGAGAAACA X GUACAUUACCUGGUA | UCAC CGAC CCCUCC |
| 6939 | CUGAAG AGAA GGCC ACCAGAGAAACA X GUACAUUACCUGGUA | GGCC AGCU CUUCAG |
| 7007 | GUCAGG AGAA GGGG ACCAGAGAAACA X GUACAUUACCUGGUA | CCCC GGAC GCUGAC |
| 7013 | GAUGAG AGAA GGGU ACCAGAGAAACA X GUACAUUACCUGGUA | ACGC UGAC CUCAUC |
| 7114 | GCUCGA AGAA GGUC ACCAGAGAAACA X GUACAUUACCUGGUA | GACC CGCU UCGAGC |
| 7148 | UGGUGC AGAA GAUA ACCAGAGAAACA X GUACAUUACCUGGUA | UAUC CGUU GCACCA |
| 7214 | GUUGUA AGAA GGGC ACCAGAGAAACA X GUACAUUACCUGGUA | GCCC GGAU UACAAC |
| 7253 | GACGUA AGAA GGAC ACCAGAGAAACA X GUACAUUACCUGGUA | GUCC GGAC UACGUC |
| 7291 | GUGGUA AGAA GCAA ACCAGAGAAACA X GUACAUUACCUGGUA | UUGC CGCC UACCAC |
| 7315 | CGUGGA AGAA GUAU ACCAGAGAAACA X GUACAUUACCUGGUA | AUAC CGCC UCCACG |
| 7337 | CAGAAC AGAA GUCC ACCAGAGAAACA X GUACAUUACCUGGUA | GGAC GGUU GUUCUG |
| 7367 | CGCCAA AGAA GAAG ACCAGAGAAACA X GUACAUUACCUGGUA | CUUC UGCC UUGGCG |
| 7401 | AUCCGG AGAA GCCG ACCAGAGAAACA X GUACAUUACCUGGUA | CGGC AGCU CCGGAU |
| 7407 | CCGACG AGAA GGAG ACCAGAGAAACA X GUACAUUACCUGGUA | CUCC GGAU CGUCGG |
| 7415 | GUCAAC AGAA GACG ACCAGAGAAACA X GUACAUUACCUGGUA | CGUC GGCC GUUGAC |
| 7418 | GGUGUC AGAA CCCG ACCAGAGAAACA X GUACAUUACCUGGUA | CGGC CGUU GACAGC |
| 7439 | GGGAGG AGAA GUCG ACCAGAGAAACA X GUACAUUACCUGGUA | CGAC CGGC CCUCCC |
| 7448 | GGUCUG AGAA GGAG ACCAGAGAAACA X GUACAUUACCUGGUA | CUCC CGAU CAGACC |
| 7453 | UCAGAG AGAA GAUC ACCAGAGAAACA X GUACAUUACCUGGUA | GAUC AGAC CUCUGA |
| 7460 | ACCGUC AGAA GAGG ACCAGAGAAACA X GUACAUUACCUGGUA | CCUC UGAC GACGGU |
| 7481 | CUCAAC AGAA GAUU ACCAGAGAAACA X GUACAUUACCUGGUA | AAUC UGAC GUUGAG |
| 7535 | GCUGAG AGAA GGGU ACCAGAGAAACA X GUACAUUACCUGGUA | ACCC UGAU CUCAGC |
| 7593 | UUGAGC AGAA GACG ACCAGAGAAACA X GUACAUUACCUGGUA | CGUC UGCU GCUCAA |
| 7596 | ACAUUG AGAA GCAG ACCAGAGAAACA X GUACAUUACCUGGUA | CUGC UGCU CAAUGU |
| 7627 | GGCGUG AGAA GGGC ACCAGAGAAACA X GUACAUUACCUGGUA | GCCC UGAU CACGCC |
| 7660 | UUGAUG AGAA GCUU ACCAGAGAAACA X GUACAUUACCUGGUA | AAGC UGCC CAUCAA |
| 7687 | UGACGC AGAA GAGA ACCAGAGAAACA X GUACAUUACCUGGUA | UCUC UGCU GCGUCA |
| 7764 | CUUGCA AGAA GUCA ACCAGAGAAACA X GUACAUUACCUGGUA | UGAC AGAC UGCAAG |
| 7870 | GGGGGC AGAA GCUU ACCAGAGAAACA X GUACAUUACCUGGUA | AAGC UGAC GCCCCC |
| 7956 | ACACGG AGAA GAUG ACCAGAGAAACA X GUACAUUACCUGGUA | CAUC CGCU CCGUGU |
| 7975 | UCUUCC AGAA GGUC ACCAGAGAAACA X GUACAUUACCUGGUA | GACC UGCU GGAAGA |
| 8066 | AAGGCG AGAA GGCU ACCAGAGAAACA X GUACAUUACCUGGUA | AGCC AGCU CGCCUU |
| 8087 | UCCCAG AGAA GGGA ACCAGAGAAACA X GUACAUUACCUGGUA | UCCC AGAC CUGGGA |
| 8172 | ACUGGA AGAA GUAC ACCAGAGAAACA X GUACAUUACCUGGUA | GUAC GGAU UCCAGU |
| 8262 | CAAAGC AGAA GGUG ACCAGAGAAACA X GUACAUUACCUGGUA | CACC CGCU GCUUUG |
| 8265 | AGUCAA AGAA GCGG ACCAGAGAAACA X GUACAUUACCUGGUA | CCGC UGCU UUGACU |
| 8374 | AUGUAG AGAA GCUC ACCAGAGAAACA X GUACAUUACCUGGUA | GAGC GGCU CUACAU |
| 8395 | GAAUUA AGAA GGGG ACCAGAGAAACA X GUACAUUACCUGGUA | CCCC UGAC UAAUUC |
| 8452 | CUAGUC AGAA GCAC ACCAGAGAAACA X GUACAUUACCUGGUA | GUGC UGAC GACUAG |
| 8501 | UCGACA AGAA GCAG ACCAGAGAAACA X GUACAUUACCUGGUA | CUGC GGCC UGUCGA |
| 8505 | CAGCUC AGAA GGCC ACCAGAGAAACA X GUACAUUACCUGGUA | GGCC UGUC GAGCUG |
| 8639 | GGGGGG AGAA GAGG ACCAGAGAAACA X GUACAUUACCUGGUA | ACUC UGCC CCCCCC |
| 8656 | GGUUGG AGAA GGUC ACCAGAGAAACA X GUACAUUACCUGGUA | GACC CGCC CCAACC |
| 8711 | GUGCGC AGAA GACA ACCAGAGAAACA X GUACAUUACCUGGUA | UGUC GGUC GCGCAC |
| 8911 | UUUUCA AGAA GUUC ACCAGAGAAACA X GUACAUUACCUGCUA | GAAC AGCU UGAAAA |
| 8935 | CCGUAG AGAA GACA ACCAGAGAAACA X GUACAUUACCUGGUA | UGUC AGAU CUACGG |
| 8980 | UGAAUG AGAA GAGG ACCAGAGAAACA X GUACAUUACCUGGUA | CCUC AGAU CAUUCA |
| 9082 | CGCAAG AGAA GUAC ACCAGAGAAACA X GUACAUUACCUGGUA | GUAC CGCC CUUGCG |
| 9133 | CCUUGG AGAA GUAG ACCAGAGAAACA X GUACAUUACCUGCUA | CUAC UGUC CCAAGG |
| 9218 | GGACGC AGAA GGGA ACCAGAGAAACA X GUACAUUACCUGGUA | UCCC GGCC GCGUCC |
| 9229 | AAGUCC AGAA GGGA ACCAGAGAAACA X GUACAUUACCUGGUA | UCCC AGCU GGACUU |
| 9243 | CGAACC AGAA GGAC ACCAGAGAAACA X GUACAUUACCUGGUA | GUCC AGCU GGUUCG |
| 9285 | GAGACA AGAA GUGA ACCAGAGAAACA X GUACAUUACCUGGUA | UCAC AGCC UGUCUC |
| 9289 | GCACGA AGAA GGCU ACCAGAGAAACA X GUACAUUACCUGGUA | AGCC UGUC UCGUGC |
| 9300 | AGCGGG AGAA GGCA ACCAGAGAAACA X GUACAUUACCUGGUA | UGCC CGAC CCCGCU |
| 9306 | UAAACC AGAA GGGU ACCAGAGAAACA X GUACAUUACCUGGUA | ACCC CGCU GGUUUA |
| 9358 | UUGGGG AGAA GGUA ACCAGAGAAACA X GUACAUUACCUGGUA | UACC UGCU CCCCAA |

Where “X” represents stem IV region of a HP ribozyme (Berzal-Herranz et al., 1993, EMBO.J. 12, 2567). The length of stem IV may be ≧2 base-pairs.

TABLE VIII

Additional HCV Conserved Hammerhead ribozyme and target sequence

| | | | | |
|---|---|---|---|---|
| Nos. | Name* | Pos.† | Ribozyme | Substrate |

| | | | | |
|---|---|---|---|---|
| 1 | HCV.C- 48 | 278 | UUGGUGU CUGAUGAG X CGAA ACGUUUG | CAAACGU A ACACCAA |
| 2 | HCV.C- 60 | 290 | UGUGGGC CUGAUGAG X CGAA ACGGUUG | CAACCGU C GCCCACA |
| 3 | HCV.C-175 | 405 | AGGUUGU CUGAUGAG X CGAA ACCGCUC | GAGCGGU C ACAACCU |
| 4 | HCV.3-118 | 9418 | AAAAAAA CUGAUGAG X CGAA AAAAAAA | UUUUUUU U UUUUUUU |
| 5 | HCV.3-145 | 9445 | UAAGAUG CUGAUGAG X CGAA AGCCACC | GGUGGCU C CAUCUUA |
| 6 | HCV.3-149 | 9449 | GGGCUAA CUGAUGAG X CGAA AUGGAGC | GCUCCAU C UUAGCCC |
| 7 | HCV.3-151 | 9451 | UAGGGCU CUGAUGAG X CGAA AGAUGGA | UCCAUCU U AGCCCUA |
| 8 | HCV.3-152 | 9452 | CUAGGGC CUGAUGAG X CGAA AAGAUGG | CCAUCUU A GCCCUAG |
| 9 | HCV.3-158 | 9458 | CCGUGAC CUGAUGAG X CGAA AGGGCUA | UAGCCCU A GUCACGG |
| 10 | HCV.3-161 | 9461 | UAGCCGU CUGAUGAG X CGAA ACUAGGG | CCCUAGU C ACGGCUA |
| 11 | HCV.3-168 | 9468 | UCACAGC CUGAUGAG X CGAA AGCCGUG | CACGGCU A GCUGUGA |
| 12 | HCV.3-181 | 9481 | GCUCACG CUGAUGAG X CGAA ACCUUUC | GAAAGGU C CGUGAGC |

Where “X” represents stem II region of a HH ribozyme (Hertel et al., 1992 Nucleic Acids Res. 20:3252). The length of stem II may be ≧2 base-pairs.
Core Reference Sequence for Nos. 1-3 = HPCCOPR (Acc#L38318) 1-600 bp
*Nucleotide 231 (8 nucleotide upstream of the initiator ATG) has been designated as “1” for the purpose of numbering ribozyme sites in the core protein coding region.
3′-NCR Reference Sequence for Nos. 4-12 = D85516 (Acc#D85516) 9301-9535 bp
*Nucleotide 9301 has been designated as “1” for the purpose of numbering ribozyme sites in the 3′NCR.
†position number reflects the reference sequence from HPCCOPR.

TABLE IX
Inhibition of HCV RNA in OST7 cells Using Mulitiple Ribozyme Motifs

| Motif | RPI Number | F_{Iuc}/R_{Iuc} | SEM | Sequence |
|---|---|---|---|---|
| RPI Motif I | Irrelevant Control | 0.22 | 0.03 | auccuUGAUₛGGCAUACACUAUGCGCGaugaucugcaB |
| RPI Motif I | 18738 | 0.13 | 0.03 | acacuuGAUₛggcauGcacuaugcgcgauacuaacgcB |
| RPI Motif I | 18739 | 0.15 | 0.01 | cacgauGAUₛggcauocacuaugcgcgacucauacUaB |
| RPI Motif I | 18740 | 0.15 | 0.01 | ggcuguGAUₛggcauGcacuaugcgcgacgacacucaB |
| RPI Motif I | 18746 | 0.10 | 0.02 | cccaauGAUₛggcauGcacuaugcgcgaCuaCUCggCB |
| RPI Motif I | 18747 | 0.16 | 0.02 | uuucguGAUₛggcauGcacuaugcgcggacccaaCaCB |
| RPI Motif I | 18750 | 0.15 | 0.03 | ucagguGAUₛggcauGcacuaugcgcgaguaccacaaB |
| RPI Motif I | 18754 | 0.12 | 0.01 | gcacuuGAUₛggcauGcacuaugcgcggcaagcacccB |
| RPI Motif II | SAC | 1.10 | 0.32 | aₛuₛuₛcₛca cUAGuGaggcguuagccGau Acgcga B |
| RPI Motif II | 20339 | 0.85 | 0.01 | uₛcₛcₛuₛcaccUGAuGaggccguuaggccGaaIgggaguB |
| RPI Motif II | 20350 | 1.04 | 0.05 | gₛuₛcₛcₛuggcUGAuGaggccguuaggccGaaIgcugcaB |
| RPI Motif III | Irrelevant Control | 1.28 | 0.07 | ggaaaggugugcaaCCGgaggaaacucCCUUCAAGGACAUCGUCCGGGacggcB |
| RPI Motif III | 18704 | 0.37 | 0.07 | uuccgcagaCGgaggaaacucCCUUCAAGGACGAAAGUCCGGGacuauggB |
| RPI Motif III | 18705 | 0.42 | 0.10 | ccgcagaCGgaggaaacucCCUUCAAGGACGAAAGUCCGGGacuauggB |
| RPI Motif III | 18700 | 0.61 | 0.16 | cagguaguaCGgaggaaacucCCUUCAAGGACAUCGUCCGGGacaaggB |
| RPI Motif III | 18701 | 0.54 | 0.10 | gcacggucUaGgaggaaacucCCUUCAAGGACAUCGUCCGGGgagaccB |
| RPI Motif III | 18835 | 0.54 | 0.04 | guguacucacGgaggaaacucCCUUCAAGGACAUCGUCCGGGgguucB |

Chemical Modifications are indicated as follows:
Lower case = 2′-O-Methyl
Bold (non-italicized): 2′-NH₂
U = 2′-C-Allyl-U
G, A = ribo G, A
s = phosphorothioate linkages
B = inverted abasic
I = ribo Inosine

1. An enzymatic nucleic acid molecule which specifically cleaves RNA derived from hepatitis C virus (HCV), wherein said enzymatic nucleic acid molecule is in a hammerhead motif, wherein the binding arms of said enzymatic nucleic acid molecule comprises sequences complementary to any of substrate sequences defmed in tables IV-VI and VIII.

2. An enzymatic nucleic acid molecule which specifically cleaves RNA derived from hepatitis C virus (HCV), wherein said enzymatic nucleic acid molecule is in a hairpin motif, wherein the binding arms of said enzymatic nucleic acid molecule comprises sequences complementary to any of substrate sequences defmed in table VII.

3. The enzymatic nucleic acid molecule of claim 1, wherein said enzymatic nucleic acid molecule comprises a stem II region of length greater than or equal to 2 base pairs.

4. The enzymatic nucleic acid molecule of claims 1 or 2, wherein said nucleic acid comprises between 12 and 100 bases complementary to said RNA.

5. The enzymatic nucleic acid molecule of claim 1 or 2, wherein said nucleic acid comprises between 14 and 24 bases complementary to said mRNA.

6. The enzymatic nucleic acid of claim 2, wherein said enzymatic nucleic acid molecule comprises a stem II region of length between three and seven base-pairs.

7. The enzymatic nucleic acid molecule of claim 2, wherein said enzymatic nucleic acid molecule consists essentially of any ribozyme sequence defined in Table VII.

8. The enzymatic nucleic acid molecule of claim 1, wherein said enzymatic nucleic acid molecule consists essentially of any ribozyme sequence defined in Tables IV-VI and VIII.

9. A pharmaceutical composition comprising the enzymatic nucleic acid molecule of claims 1 or 2.

10. A mammalian cell including an enzymatic nucleic acid molecule of any of claims 1 or 2.

11. The mammalian cell of claim 10, wherein said mammalian cell is a human cell.

12. An expression vector comprising nucleic acid sequence encoding at least one enzymatic nucleic acid molecule of claims 1 or 2, in a manner which allows expression of that enzymatic nucleic acid molecule.

13. A mammalian cell including an expression vector of claim 12.

14. The mammalian cell of claim 13, wherein said mammalian cell is a human cell.

15. A method for treatment of cirrhosis, liver failure or hepatocellular carcinoma comprising the step of administering to a patient the enzymatic nucleic acid molecule of claims 1 or 2 under conditions suitable for said treatment.

16. A method for treatment of cirrhosis, liver failure and/or hepatocellular carcinoma comprising the step of administering to a patient the expression vector of claims 1 or 2 under conditions suitable for said treatment.

17. A method of treatment of a patient having a condition associated with HCV infection, comprising contacting cells of said patient with the nucleic acid molecule of claims 1 or 2, and further comprising the use of one or more drug therapies under conditions suitable for said treatment.

18. The enzymatic nucleic acid molecule of claim 1, wherein said nucleic acid molecule comprises at least five ribose residues, and wherein said nucleic acid comprises phosphorothioate linkages at at least three of the 5′ terminal nucleotides, and wherein said nucleic acid comprises a 2′-C-allyl modification at position No. 4 of said nucleic acid, and wherein said nucleic acid comprises at least ten 2′-O-methyl modifications, and wherein said nucleic acid comprises a 3′-end modification.

19. The enzymatic nucleic acid of claim 18, wherein said nucleic acid comprises a 3′-3′ linked inverted abasic moiety at said 3′ end.

20. The enzymatic nucleic acid molecule of claim 1, wherein said nucleic acid molecule comprises at least five ribose residues, and wherein said nucleic acid molecule comprises phosphorothioate linkages at at least three of the 5′ terminal nucleotides, and wherein said nucleic acid comprises a 2′-amino modification at position No. 4 and/or at position No. 7 of said nucleic acid molecule, wherein said nucleic acid molecule comprises at least ten 2′-O-methyl modifications, and wherein said nucleic acid comprises a 3′-end modification.

21. The enzymatic nucleic acid molecule of claim 1, wherein said nucleic acid molecule comprises at least five ribose residues, and wherein said nucleic acid molecule comprises phosphorothioate linkages at at least three of the 5′ terminal nucleotides, and wherein said nucleic acid molecule comprises an abasic substitution at position No. 4 and/or at position No. 7 of said nucleic acid molecule, wherein said nucleic acid comprises at least ten 2′-O-methyl modifications, and wherein said nucleic acid molecule comprises a 3′-end modification.

22. The enzymatic nucleic acid molecule of claim 1, wherein said nucleic acid molecule comprises of at least five ribose residues, and wherein said nucleic acid comprises phosphorothioate linkages at at least three of the 5′ terminal nucleotides, and wherein said nucleic acid molecule comprises a 6-methyl uridine substitution at position No. 4 and/or at position No. 7 of said nucleic acid molecule, wherein said nucleic acid molecule comprises at least ten 2′-O-methyl modifications, and wherein said nucleic acid molecule comprises a 3′ end modification.

23. A method for inhibiting HCV replication in a mammalian cell comprising the step of administering to said cell the enzymatic nucleic acid molecule of claims 1 or 2 under conditions suitable for said inhibition.

24. A method of cleaving a separate RNA molecule comprising, contacting the enzymatic nucleic acid molecule of claims 1 or 2 with said separate RNA molecule under conditions suitable for the cleavage of said separate RNA molecule.

25. The method of claim 24, wherein said cleavage is carried out in the presence of a divalent cation.

26. The method of claim 25, wherein said divalent cation is Mg²⁺.

27. The nucleic acid molecule of claim 1 or 2, wherein said nucleic acid is chemically synthesized.

28. The expression vector of claim 12, wherein said vector comprises:
a) a transcription initiation region;
b) a transcription termination region;
c) a gene encoding at least one said nucleic acid molecule; and
wherein said gene is operably linked to said initiation region and said termination region, in a manner which allows expression and/or delivery of said nucleic acid molecule.

29. The expression vector of claim 12, wherein said vector comprises:
a) a transcription initiation region;
b) a transcription termination region;
c) an open reading frame;
d) a gene encoding at least one said nucleic acid molecule, wherein said gene is operably linked to the 3′-end of said open reading frame; and
wherein said gene is operably linked to said initiation region, said open reading frame and said termination region, in a manner which allows expression and/or delivery of said nucleic acid molecule.

30. The expression vector of claim 12, wherein said vector comprises:
a) a transcription initiation region;
b) a transcription termination region;
c) an intron;
d) a gene encoding at least one said nucleic acid molecule; and
wherein said gene is operably linked to said initiation region, said intron and said termination region, in a manner which allows expression and/or delivery of said nucleic acid molecule.

31. The expression vector of claim 12, wherein said vector comprises:
a) a transcription initiation region;
b) a transcription termination region;
c) an intron;
d) an open reading frame;
e) a gene encoding at least one said nucleic acid molecule, wherein said gene is operably linked to the 3′-end of said open reading frame; and wherein said gene is operably linked to said initiation region, said intron, said open reading frame and said termination region, in a manner which allows expression and/or delivery of said nucleic acid molecule.

32. An enzymatic nucleic acid molecule which specifically cleaves RNA derived from hepatitis C virus (HCV), wherein said enzymatic nucleic acid molecule is a DNA enzyme.

33. The enzymatic nucleic acid molecule of any of claims 1, 2 or 32, wherein said enzymatic nucleic acid comprises at least one 2′-sugar modification.

34. The enzymatic nucleic acid molecule of any of claims 1, 2 or 32, wherein said enzymatic nucleic acid comprises at least one nucleic acid base modification.

35. The enzymatic nucleic acid molecule of any of claims 1, 2 or 32, wherein said enzymatic nucleic acid comprises at least one phosphorothioate modification.

36. The method of claim 17, wherein said drug therapies is type I interferon.

37. The method of claim 36, wherein said type I interferon and the enzymatic nucleic acid molecule is administered simultaneously.

38. The method of claim 36, wherein said type I interferon and enzymatic nucleic acid molecule is administered separately.

39. The method of claim 36, wherein said type I interferon is interferon alpha.

40. The method of claim 36, wherein said type I interferon is interferon beta.

41. The method of claim 36, wherein said type I interferon is interferon gamma.

42. The method of claim 36, wherein said type I interferon is consensus interferon.

43. A method of treatment of a patient having a condition associated with HCV infection, comprising contacting cells of said patient with the nucleic acid molecule of claims 32, and further comprising the use of one or more drug therapies under conditions suitable for said treatment.

44. The method of claim 43, wherein said drug therapies is type I interferon.

45. The method of claim 44, wherein said type I interferon and the enzymatic nucleic acid molecule is administered simultaneously.

46. The method of claim 44, wherein said type I interferon and enzymatic nucleic acid molecule is administered separately.

47. The method of claim 44, wherein said type I interferon is interferon alpha.

48. The method of claim 44, wherein said type I interferon is interferon beta.

49. The method of claim 44, wherein said type I interferon is interferon gamma.

50. The method of claim 44, wherein said type I interferon is consensus interferon.
